(19) United States
(12) Patent Application Publication
(10) Pub. No.: US 2023/0157157 A1
(43) Pub. Date: May 18, 2023

(54) LIGHT-EMITTING DEVICE INCLUDING DIAMINE-BASED COMPOUND, ELECTRONIC APPARATUS INCLUDING THE LIGHT-EMITTING DEVICE, AND THE DIAMINE-BASED COMPOUND

(71) Applicant: Samsung Display Co., Ltd., Yongin-si (KR)

(72) Inventors: Eunjae Jeong, Yongin-si (KR); Minji Kim, Yongin-si (KR); Hyunbin Park, Yongin-si (KR); Jeongmin Lee, Yongin-si (KR); Jiyong Choi, Yongin-si (KR); Sanghyun Han, Yongin-si (KR)

(21) Appl. No.: 17/984,140

(22) Filed: Nov. 9, 2022

(30) Foreign Application Priority Data
Nov. 12, 2021 (KR) 10-2021-0156049

(51) Int. Cl. H01L 51/00; C07C 211/61; C09K 11/06; C07D 307/91; C07D 333/76

(52) U.S. Cl. CPC H01L 51/006; C07C 211/61; C09K 11/06; C07D 307/91; H01L 51/0061; C07D 333/76; H01L 51/5064

(57) ABSTRACT

A light-emitting device that includes a diamine-based compound represented by Formula 1, and an electronic apparatus that includes the light-emitting device are provided.

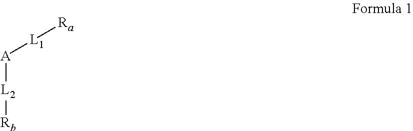

### CROSS-REFERENCE TO RELATED APPLICATION

This application claims priority to and the benefit of Korean Patent Application No. 10-2021-0156049, filed on Nov. 12, 2021, in the Korean Intellectual Property Office, the entire content of which is hereby incorporated by reference herein.

### BACKGROUND

### 1. Field

One or more embodiments of the present disclosure relate to a light-emitting device including a diamine-based compound, an electronic apparatus including the light-emitting device, and the diamine-based compound.

### 2. Description of the Related Art

Self-emissive devices among light-emitting devices have wide viewing angles, high contrast ratios, short response times, and excellent characteristics in terms of luminance, driving voltage, and response speed.

In a light-emitting device, a first electrode is arranged on a substrate, and a hole transport region, an emission layer, an electron transport region, and a second electrode are sequentially arranged on the first electrode. Holes provided from the first electrode may move toward the emission layer through the hole transport region, and electrons provided from the second electrode may move toward the emission layer through the electron transport region. Carriers, such as holes and electrons, recombine in an emission layer region to produce excitons. These excitons may transition from an excited state to a ground state, thereby generating light.

### SUMMARY

Provided are a light-emitting device including a diamine-based compound, and an electronic apparatus that includes the light-emitting device, which includes the diamine-based compound.

Additional aspects of embodiments of the present disclosure will be set forth in part in the description, which follows and, in part, will be apparent from the disclosure, or may be learned by practice of the presented embodiments of the disclosure.

According to one or more embodiments, provided is a light-emitting device including

a first electrode,

a second electrode facing the first electrode,

an interlayer arranged between the first electrode and the second electrode and including an emission layer (in the interlayer), and

a diamine-based compound represented by Formula 1.

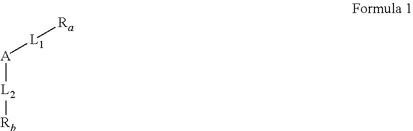

In Formula 1,

A may be or

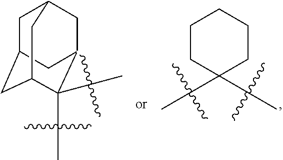 [SMILES: *C1(*)C2CC3CC(C2)CC1C3 ; *C1(*)CCCCC1]

L₁ and L₂ may each independently be a single bond, *—C(R₅)(R₆)—*′, *—C(R₅)═*′, *═C(R₅)—*′, *—C(R₅)═C(R⁶)—*′, *—C(═O)—*′, *—C(═S)—*′, *—C≡C—*′, *—B(R)—*′, *—N(R₅)—*′, *—P(R₅)—*′, *—Si(R₅)(R₆)—*′, *—P(═O)(R₅)—*′, *—S(═O)—*′, *—S(═O)₂—*′, or *—Ge(R₅)(R⁶)—*′,

in Formula 1, [symbol] , *, and *′ each indicate a binding site to a neighboring atom, and

[symbol]

Rₐ and R_{b} may each independently be a group represented by Formula 1-1,

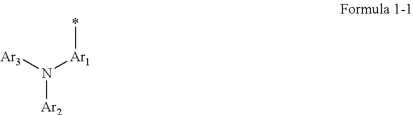

wherein, in Formula 1-1,

Ar₁ may be a divalent linking group of a C₆-C₆₀ aryl group unsubstituted or substituted with at least one R₁₀ₐ or a divalent linking group of a C₃-C₆₀ heteroaryl group unsubstituted or substituted with at least one R₁₀ₐ, and * indicates a binding site to an atom included in A,

Ar₂ and Ar₃ may each independently be a C₆-C₆₀ aryl group unsubstituted or substituted with at least one R₁₀ₐ or a C₃-C₆₀ heteroaryl group unsubstituted or substituted with at least one R₁₀ₐ, and

at least one group of Ar₂ and Ar₃ may be a group represented by Formula 1-2,

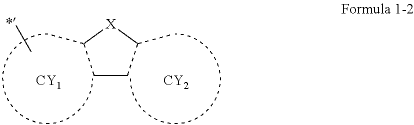

wherein X may be one of O, S, N(Q₁), P(Q₁), C(Q₁)(Q₂), and Si(Q₁)(Q₂),

CY₁ and CY₂ may each independently be a C₆-C₃₀ aryl group unsubstituted or substituted with at least one R₁₀ₐ or a C₃-C₃₀ heteroaryl group unsubstituted or substituted with at least one R₁₀ₐ,

*′ indicates a binding site to an atom included in the group represented by Formula 1-1,

R₁₀ₐ may be

deuterium, —F, —Cl, —Br, —I, a hydroxyl group, a cyano group, or a nitro group,

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any suitable combinations thereof,

a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any suitable combinations thereof, or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

Q₁, Q₂, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any suitable combinations thereof, and

Q₁ and Q₂ may optionally be bonded together to form a C₃-C₆₀ carbocyclic group unsubstituted or substituted with at least one R₁₀ₐ or a C₁-C₆₀ heterocyclic group unsubstituted or substituted with at least one R₁₀ₐ.

According to one or more embodiments, provided is an electronic apparatus including the light-emitting device.

According to one or more embodiments, provided is the diamine-based compound represented by Formula 1.

### BRIEF DESCRIPTION OF THE DRAWINGS

The above and other aspects and features of certain embodiments of the disclosure will be more apparent from the following description taken in conjunction with the accompanying drawings, in which:

FIG. 1 shows a schematic view of a structure of a light-emitting device according to an embodiment;

FIG. 2 shows a schematic view of a structure of an electronic apparatus according to an embodiment; and

FIG. 3 shows a schematic view of an electronic apparatus according to another embodiment.

### DETAILED DESCRIPTION

Reference will now be made in more detail to embodiments, examples of which are illustrated in the accompanying drawings, wherein like reference numerals refer to like elements throughout the disclosure, and duplicative descriptions thereof may not be provided. In this regard, the present embodiments may have different forms and should not be construed as being limited to the descriptions set forth herein. Accordingly, the embodiments are merely described below, by referring to the figures, to explain aspects of embodiments of the present disclosure. As used herein, the term “and/or” includes any and all combinations of one or more of the same associated listed items. Throughout the disclosure, the expression “at least one of a, b or c” indicates only a, only b, only c, both a and b, both a and c, both b and c, all of a, b, and c, or variations thereof.

A light-emitting device of the disclosure may include: a first electrode; a second electrode facing the first electrode; an interlayer arranged between the first electrode and the second electrode and including an emission layer (in the interlayer); and a diamine-based compound represented by Formula 1.

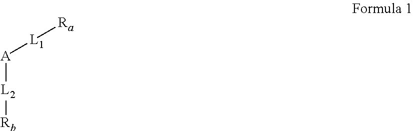

In Formula 1,

A may be

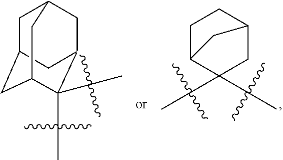 [SMILES: *C1(*)C2CC3CC(C2)CC1C3 ; *C1(*)CC2CCC1C2]

L₁ and L₂ may each independently be a single bond, *—C(R₅)(R₆)—*′, *—C(R₅)═*′, *═C(R₅)—*′, *—C(R₅)═C(R₆)—*′, *—C(═O)—*, *—C(═S)—*′, *—C≡C—*′, *—B(R₅)—*′, *—N(R₅)—*′, *—P(R₅)—*′, *—Si(R₅)(R₆)—*′, *—P(═O)(R₅)—*′, *—S(═O)—*′, *—S(═O)₂—*′, or *—Ge(R₅)(R⁶)—*′,

in Formula 1, [symbol] , *, and *′ each indicate a binding site to a neighboring atom, and

[symbol]

Rₐ and R_{b} may each independently be a group represented by Formula 1-1,

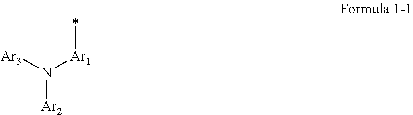

wherein, in Formula 1-1,

Ar₁ may be a divalent linking group of a C₆-C₆₀ aryl group unsubstituted or substituted with at least one R₁₀ₐ or a divalent linking group of a C₃-C₆₀ heteroaryl group unsubstituted or substituted with at least one R₁₀ₐ, and * indicates a binding site to an atom included in A,

Ar₂ and Ar₃ may each independently be a C₆-C₆₀ aryl group unsubstituted or substituted with at least one R₁₀ₐ or a C₃-C₆₀ heteroaryl group unsubstituted or substituted with at least one R₁₀ₐ, and

at least one group of Ar₂ and Ar₃ may be a group represented by Formula 1-2,

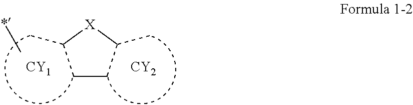

wherein X may be one of O, S, N(Q₁), P(Q₁), C(Q₁)(Q₂), and Si(Q₁)(Q₂),

CY₁ and CY₂ may each independently be a C₆-C₃₀ aryl group unsubstituted or substituted with at least one R₁₀ₐ or a C₃-C₃₀ heteroaryl group unsubstituted or substituted with at least one R₁₀ₐ,

*′ indicates a binding site to an atom included in the group represented by Formula 1-1,

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any suitable combinations thereof;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any suitable combinations thereof;

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

Q₁, Q₂, Q₁₁ to Q₁₃, Q₂₁ to Q₂₃, and Q₃₁ to Q₃₃ may each independently be: hydrogen; deuterium; —F; —CI; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any suitable combinations thereof, and

Q₁ and Q₂ may optionally be bonded together to form a C₃-C₆₀ carbocyclic group unsubstituted or substituted with at least one R₁₀ₐ or a C₁-C₆₀ heterocyclic group unsubstituted or substituted with at least one R₁₀ₐ.

In an embodiment, the interlayer may include the diamine-based compound represented by Formula 1.

In an embodiment, the first electrode may be an anode, the second electrode may be a cathode, the interlayer may further include a hole transport region between the first electrode and the emission layer and an electron transport region between the emission layer and the second electrode, the hole transport region may include a hole injection layer, a first hole transport layer, an emission auxiliary layer, an electron blocking layer, or any combination thereof, and the electron transport region may include a hole blocking layer, an electron transport layer, an electron injection layer, or any suitable combinations thereof.

In an embodiment, the hole transport region may include the diamine-based compound represented by Formula 1.

In an embodiment, the first hole transport layer may include the diamine-based compound represented by Formula 1.

In an embodiment, the hole transport region may further include a second hole transport layer and a third hole transport layer, the second hole transport layer may be an amine-based compound represented by N(Ar₂₁)(Ar₂₂)(Ar₂₃), the third hole transport layer may include the same diamine-based compound as the interlayer, Ar₂₁ to Ar₂₃ may each independently be a C₆-C₆₀ aryl group unsubstituted or substituted with at least one R₁₀ₐ, a C₃-C₆₀ heteroaryl group unsubstituted or substituted with at least one R₁₀ₐ, or a C₆-C₆₀ condensed polycyclic group unsubstituted or substituted with at least one R₁₀ₐ, and R₁₀ₐ is the same as described in the present disclosure.

In an embodiment, the first hole transport layer, the second hole transport layer, and the third hole transport layer may be sequentially stacked.

In an embodiment, a thickness of the first hole transport layer to a thickness of the third hole transport layer may be equal to or different from one another.

In an embodiment, a thickness of the first hole transport layer may be from about 10 μm to about 45 μm, based on 100 μm of the total thickness of a hole transport layer.

In an embodiment, a thickness of the second hole transport layer may be from about 10 μm to about 80 μm, based on 100 μm of the total thickness of a hole transport layer.

In an embodiment, a thickness of the third hole transport layer may be from about 10 μm to about 45 μm, based on 100 μm of the total thickness of a hole transport layer.

In an embodiment, a thickness of the second hole transport layer may be from about 20 μm to about 800 μm, based on 100 μm of a thickness of the first hole transport layer.

In an embodiment, a thickness of the third hole transport layer may be from about 100 μm to about 450 μm, based on 100 μm of a thickness of the first hole transport layer.

In an embodiment, a refractive index of the second hole transport layer may be greater than a refractive index of the first hole transport layer.

In an embodiment, a refractive index of the second hole transport layer may be greater than a refractive index of the first hole transport layer by about at least 0.05, for example, about at least 0.1, for example, about at least 0.2.

In an embodiment, a refractive index of the first hole transport layer may be from about 1.4 to about 1.7, for example, from about 1.45 to about 1.65, for example, from about 1.5 to about 1.6.

A refractive index of the second hole transport layer may be from about 1.8 to about 2.1, for example, from about 1.85 to about 2.05, for example, from about 1.9 to about 2.0.

In an embodiment, the amine-based compound may be Compound HT1, and is not limited to Compound HT1.

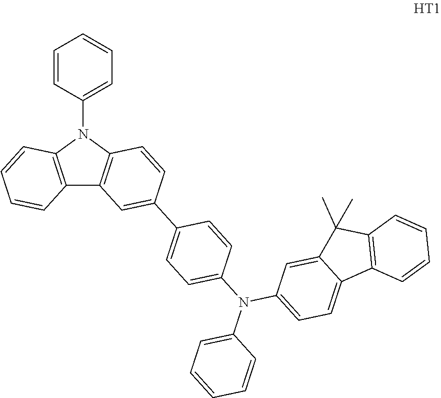 [SMILES: CC1(C)c2ccccc2-c2ccc(N(c3ccccc3)c3ccc(-c4ccc5c(c4)c4ccccc4n5-c4ccccc4)cc3)cc21]

In an embodiment, at least one of a first capping layer formed on the first electrode or a second capping layer formed on the second electrode may be further included.

In an embodiment, the first capping layer or the second capping layer may include the amine-based compound represented by Formula 1.

In an embodiment, the first capping layer or the second capping layer may further include an organic material, an inorganic material, or mixtures thereof.

In an embodiment, a refractive index of the second hole transport layer may be greater than a refractive index of the first hole transport layer by about at least 0.1, for example, about at least 0.15, for example, about at least 0.2.

In an embodiment, provided is an electron apparatus including any one of the light-emitting device embodiments.

In an embodiment, the electronic apparatus may further include a thin-film transistor, the thin-film transistor may include a source electrode and a drain electrode, and the first electrode of the light-emitting device may be electrically connected to at least one of the source electrode and the drain electrode of the thin-film transistor.

In an embodiment, the electronic apparatus may further include a color filter, a color conversion layer, a touch screen layer, a polarizing layer, or any suitable combinations thereof.

In an embodiment, provided is a diamine-based compound represented by Formula 1.

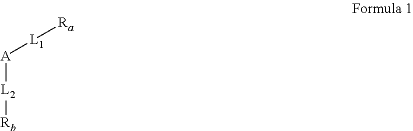

In Formula 1,

A may be

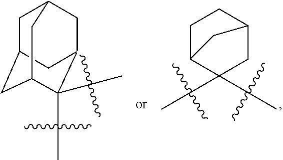 [SMILES: *C1(*)C2CC3CC(C2)CC1C3 ; *C1(*)CC2CCC1C2]

L₁ and L₂ may each independently be a single bond, *—C(R₅)(R₆)—*′, *—C(R₅)═*′, *═C(R₅)—*′, *—C(R₅)═C(R⁶)—*′, *—C(═O)—*′, *—C(═S)—*′, *—C≡C—*′, *—B(R₅)—*′, *—N(R₅)—*′, *—P(R₅)—*′, *—Si(R₅)(R₆)—*′, *—P(═O)(R₅)—*′, *—S(═O)—*′, *—S(═O)₂—*′, or *—Ge(R₅)(R⁶)—*′,

in Formula 1, [symbol] , *, and *′ each indicate a binding site to a neighboring [symbol] atom, and

Rₐ and R_{b} may each independently be a group represented by Formula 1-1,

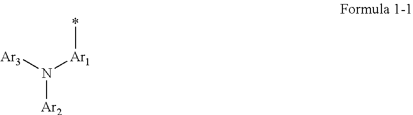

wherein, in Formula 1-1,

Ar₁ may be a divalent linking group of a C₆-C₆₀ aryl group unsubstituted or substituted with at least one R₁₀ₐ or a divalent linking group of a C₃-C₆₀ heteroaryl group unsubstituted or substituted with at least one R₁₀ₐ, and * indicates a binding site to an atom included in A,

Ar₂ and Ar₃ may each independently be a C₆-C₆₀ aryl group unsubstituted or substituted with at least one R₁₀ₐ or a C₃-C₆₀ heteroaryl group unsubstituted or substituted with at least one R₁₀ₐ, and

at least one group of Ar₂ and Ar₃ may be a group represented by Formula 1-2,

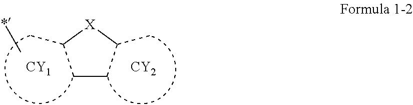

wherein X may be one of O, S, N(Q₁), P(Q₁), C(Q₁)(Q₂), and Si(Q₁)(Q₂),

CY₁ and CY₂ may each independently be a C₆-C₃₀ aryl group unsubstituted or substituted with at least one R₁₀ₐ or a C₃-C₃₀ heteroaryl group unsubstituted or substituted with at least one R₁₀ₐ,

*′ indicates a binding site to an atom included in the group represented by Formula 1-1,

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any suitable combinations thereof;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any suitable combinations thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

Q₁, Q₂, Q₁₁ to Q₁₃, Q₂₁ to Q₂₃, and Q₃₁ to Q₃₃ may each independently be: hydrogen; deuterium; —F; —CI; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any suitable combinations thereof, and

Q₁ and Q₂ may optionally be bonded together to form a C₃-C₆₀ carbocyclic group unsubstituted or substituted with at least one R₁₀ₐ or a C₁-C₆₀ heterocyclic group unsubstituted or substituted with at least one R₁₀ₐ.

In an embodiment, Rₐ and R_{b} may be different from each other.

In an embodiment, Ar₂ and Ar₃ may be different from each other.

In an embodiment, CY₁ and CY₂ may be identical to each other.

In an embodiment, a C₆-C₆₀ aryl group to a C₆-C₃₀ aryl group may each independently be one of a norbornane group, a benzene group, a pentalene group, a naphthalene group, a biphenyl group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indeno phenanthrene group, or an indenoanthracene group, and

a C₃-C₆₀ heteroaryl group to a C₃-C₃₀ heteroaryl group may each independently be one of a pyrrole group, a thiophene group, a furan group, an indole group, a benzoindole group, a naphtho indole group, an iso-indole group, a benzoiso-indole group, a naphthoiso-indole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group.

In an embodiment, a C₃-C₆₀ carbocyclic group may be a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indeno phenanthrene group, an indenoanthracene group, an adamantanyl group, a norbornanyl group, a norbornenyl group, a bicyclo[1.1.1]pentyl group, a bicyclo[2.1.1]hexyl group, a bicyclo[2.2.2]octyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.2]octyl group, a mono(C₁-C₂₀ alkyl)adamantanyl group, a di(C₁-C₂₀ alkyl)adamantanyl group, a mono(C₁-C₂₀ alkyl)norbornanyl group, a di(C₁-C₂₀ alkyl)norbornanyl group, a mono(C₁-C₂₀ alkyl)norbornenyl group, or a di(C₁-C₂₀alkyl)norbornenyl group, and

a C₁-C₆₀ heterocyclic group may be a pyrrole group, a thiophene group, a furan group, an indole group, a benzoindole group, a naphtho-indole group, an iso-indole group, a benzoiso-indole group, a naphthoiso-indole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group.

In an embodiment, at least one group of Ar₂ and Ar₃ may be a group represented by one of Formulae 1-2-1 to Formula 1-2-4.

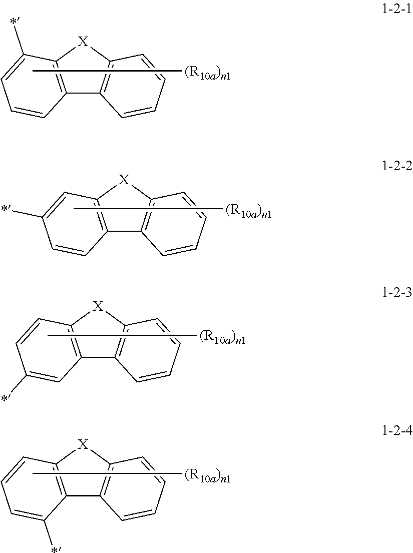

In Formulae 1-2-1 to 1-2-4, n1 may be an integer from 0 to 7, and *′, X, and R₁₀ₐ are respectively the same as those described in the present disclosure.

In an embodiment, the diamine-based compound represented by Formula 1 may be one of Compounds 1 to 88.

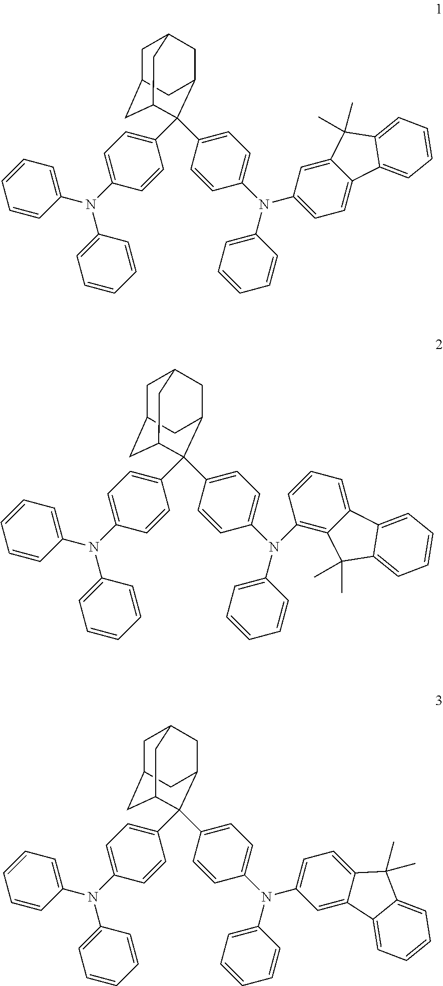 [SMILES: CC1(C)c2ccccc2-c2ccc(N(c3ccccc3)c3ccc(C4(c5ccc(N(c6ccccc6)c6ccccc6)cc5)C5CC6CC(C5)CC4C6)cc3)cc21 ; CC1(C)c2ccccc2-c2cccc(N(c3ccccc3)c3ccc(C4(c5ccc(N(c6ccccc6)c6ccccc6)cc5)C5CC6CC(C5)CC4C6)cc3)c21 ; CC1(C)c2ccccc2-c2cc(N(c3ccccc3)c3ccc(C4(c5ccc(N(c6ccccc6)c6ccccc6)cc5)C5CC6CC(C5)CC4C6)cc3)ccc21]

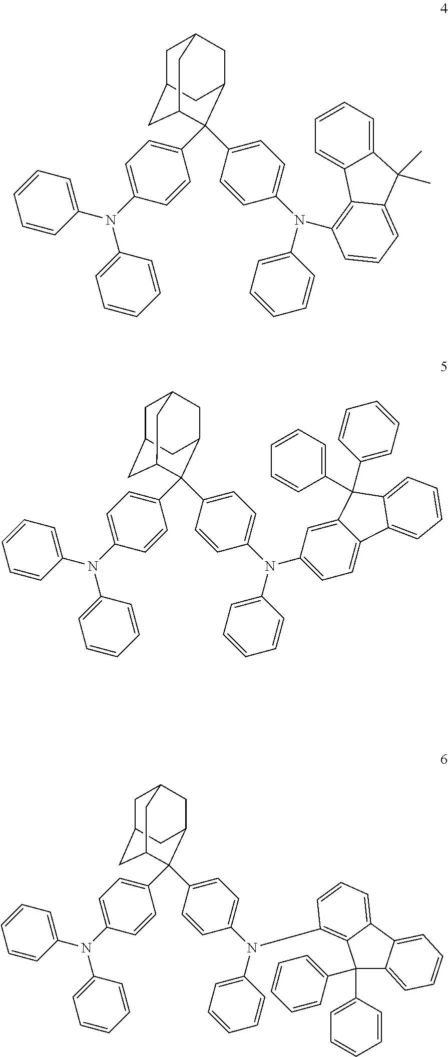 [SMILES: CC1(C)c2ccccc2-c2c(N(c3ccccc3)c3ccc(C4(c5ccc(N(c6ccccc6)c6ccccc6)cc5)C5CC6CC(C5)CC4C6)cc3)cccc21 ; c1ccc(N(c2ccccc2)c2ccc(C3(c4ccc(N(c5ccccc5)c5ccc6c(c5)C(c5ccccc5)(c5ccccc5)c5ccccc5-6)cc4)C4CC5CC(C4)CC3C5)cc2)cc1 ; c1ccc(N(c2ccccc2)c2ccc(C3(c4ccc(N(c5ccccc5)c5cccc6c5C(c5ccccc5)(c5ccccc5)c5ccccc5-6)cc4)C4CC5CC(C4)CC3C5)cc2)cc1]

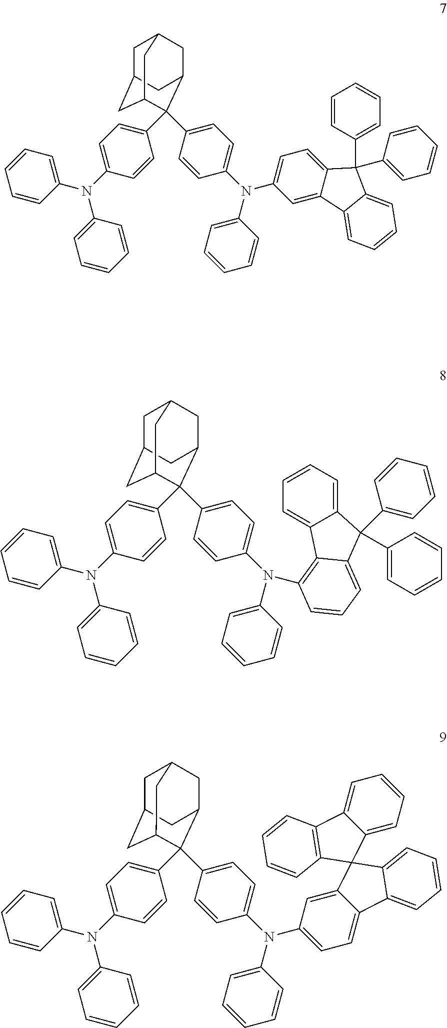 [SMILES: c1ccc(N(c2ccccc2)c2ccc(C3(c4ccc(N(c5ccccc5)c5ccc6c(c5)-c5ccccc5C6(c5ccccc5)c5ccccc5)cc4)C4CC5CC(C4)CC3C5)cc2)cc1 ; c1ccc(N(c2ccccc2)c2ccc(C3(c4ccc(N(c5ccccc5)c5cccc6c5-c5ccccc5C6(c5ccccc5)c5ccccc5)cc4)C4CC5CC(C4)CC3C5)cc2)cc1 ; c1ccc(N(c2ccccc2)c2ccc(C3(c4ccc(N(c5ccccc5)c5ccc6c(c5)C5(c7ccccc7-c7ccccc75)c5ccccc5-6)cc4)C4CC5CC(C4)CC3C5)cc2)cc1]

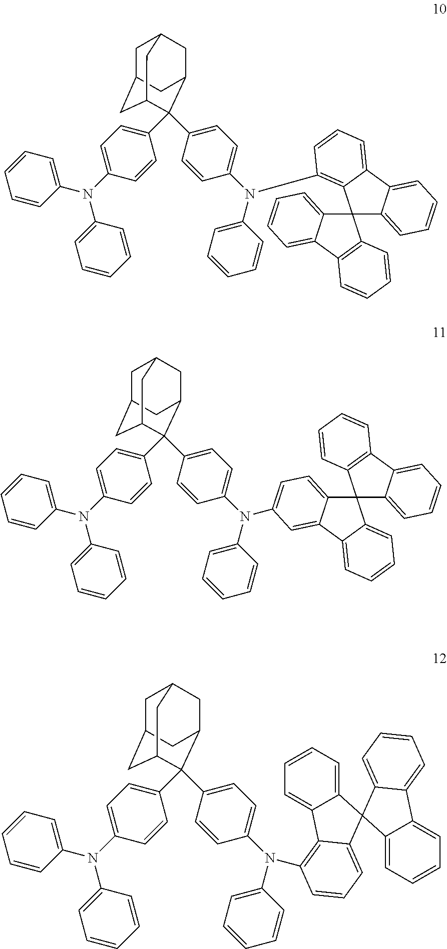 [SMILES: c1ccc(N(c2ccccc2)c2ccc(C3(c4ccc(N(c5ccccc5)c5cccc6c5C5(c7ccccc7-c7ccccc75)c5ccccc5-6)cc4)C4CC5CC(C4)CC3C5)cc2)cc1 ; c1ccc(N(c2ccccc2)c2ccc(C3(c4ccc(N(c5ccccc5)c5ccc6c(c5)-c5ccccc5C65c6ccccc6-c6ccccc65)cc4)C4CC5CC(C4)CC3C5)cc2)cc1 ; c1ccc(N(c2ccccc2)c2ccc(C3(c4ccc(N(c5ccccc5)c5cccc6c5-c5ccccc5C65c6ccccc6-c6ccccc65)cc4)C4CC5CC(C4)CC3C5)cc2)cc1]

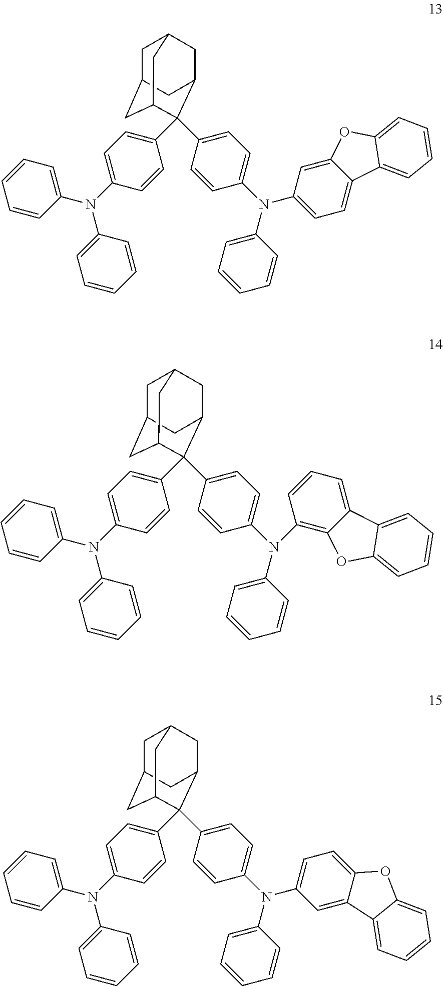 [SMILES: c1ccc(N(c2ccccc2)c2ccc(C3(c4ccc(N(c5ccccc5)c5ccc6c(c5)oc5ccccc56)cc4)C4CC5CC(C4)CC3C5)cc2)cc1 ; c1ccc(N(c2ccccc2)c2ccc(C3(c4ccc(N(c5ccccc5)c5cccc6c5oc5ccccc56)cc4)C4CC5CC(C4)CC3C5)cc2)cc1 ; c1ccc(N(c2ccccc2)c2ccc(C3(c4ccc(N(c5ccccc5)c5ccc6oc7ccccc7c6c5)cc4)C4CC5CC(C4)CC3C5)cc2)cc1]

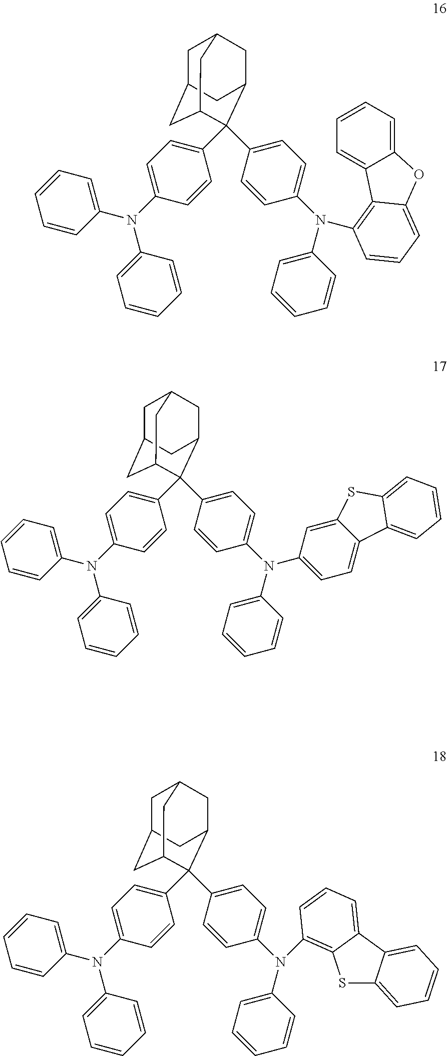 [SMILES: c1ccc(N(c2ccccc2)c2ccc(C3(c4ccc(N(c5ccccc5)c5cccc6oc7ccccc7c56)cc4)C4CC5CC(C4)CC3C5)cc2)cc1 ; c1ccc(N(c2ccccc2)c2ccc(C3(c4ccc(N(c5ccccc5)c5ccc6c(c5)sc5ccccc56)cc4)C4CC5CC(C4)CC3C5)cc2)cc1 ; c1ccc(N(c2ccccc2)c2ccc(C3(c4ccc(N(c5ccccc5)c5cccc6c5sc5ccccc56)cc4)C4CC5CC(C4)CC3C5)cc2)cc1]

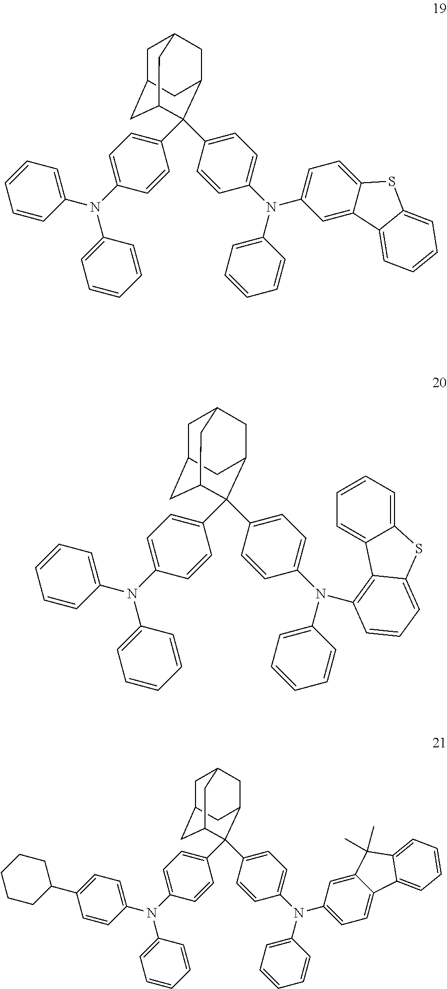 [SMILES: c1ccc(N(c2ccccc2)c2ccc(C3(c4ccc(N(c5ccccc5)c5ccc6sc7ccccc7c6c5)cc4)C4CC5CC(C4)CC3C5)cc2)cc1 ; c1ccc(N(c2ccccc2)c2ccc(C3(c4ccc(N(c5ccccc5)c5cccc6sc7ccccc7c56)cc4)C4CC5CC(C4)CC3C5)cc2)cc1 ; CC1(C)c2ccccc2-c2ccc(N(c3ccccc3)c3ccc(C4(c5ccc(N(c6ccccc6)c6ccc(C7CCCCC7)cc6)cc5)C5CC6CC(C5)CC4C6)cc3)cc21]

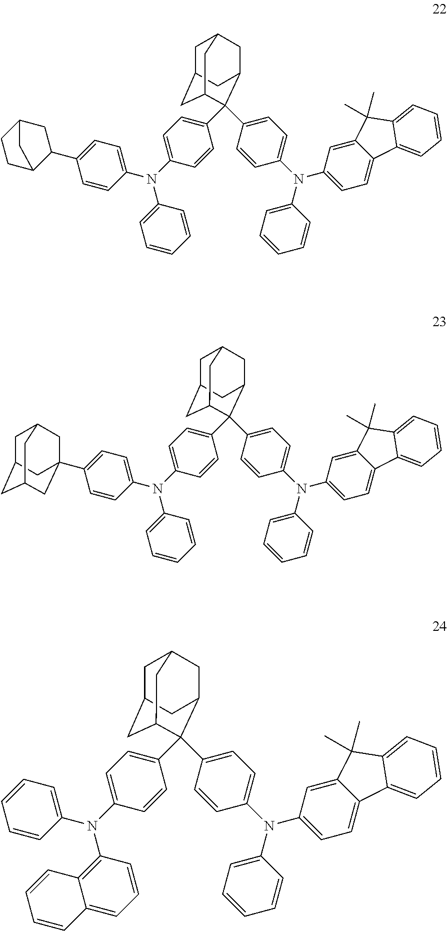 [SMILES: CC1(C)c2ccccc2-c2ccc(N(c3ccccc3)c3ccc(C4(c5ccc(N(c6ccccc6)c6ccc(C7CC8CCC7C8)cc6)cc5)C5CC6CC(C5)CC4C6)cc3)cc21 ; CC1(C)c2ccccc2-c2ccc(N(c3ccccc3)c3ccc(C4(c5ccc(N(c6ccccc6)c6ccc(C78CC9CC(CC(C9)C7)C8)cc6)cc5)C5CC6CC(C5)CC4C6)cc3)cc21 ; CC1(C)c2ccccc2-c2ccc(N(c3ccccc3)c3ccc(C4(c5ccc(N(c6ccccc6)c6cccc7ccccc67)cc5)C5CC6CC(C5)CC4C6)cc3)cc21]

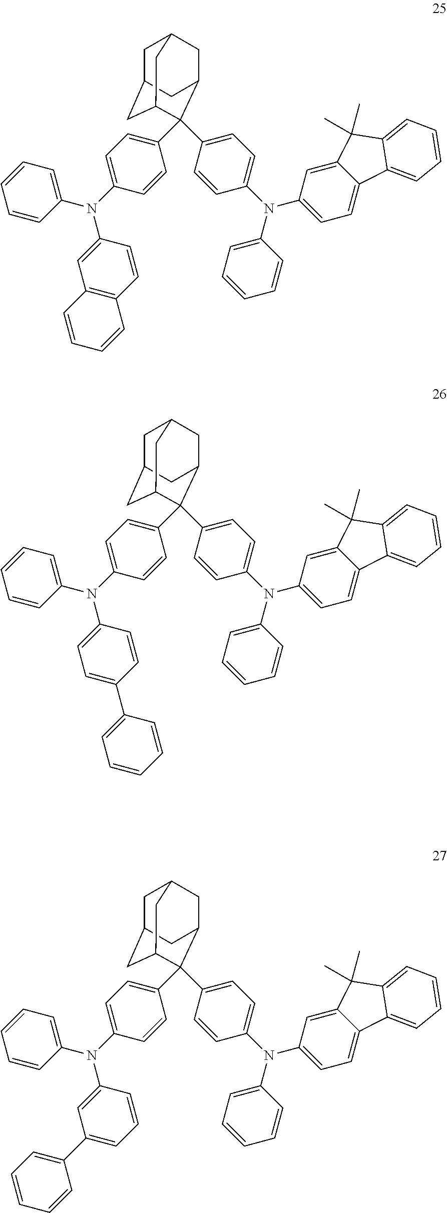 [SMILES: CC1(C)c2ccccc2-c2ccc(N(c3ccccc3)c3ccc(C4(c5ccc(N(c6ccccc6)c6ccc7ccccc7c6)cc5)C5CC6CC(C5)CC4C6)cc3)cc21 ; CC1(C)c2ccccc2-c2ccc(N(c3ccccc3)c3ccc(C4(c5ccc(N(c6ccccc6)c6ccc(-c7ccccc7)cc6)cc5)C5CC6CC(C5)CC4C6)cc3)cc21 ; CC1(C)c2ccccc2-c2ccc(N(c3ccccc3)c3ccc(C4(c5ccc(N(c6ccccc6)c6cccc(-c7ccccc7)c6)cc5)C5CC6CC(C5)CC4C6)cc3)cc21]

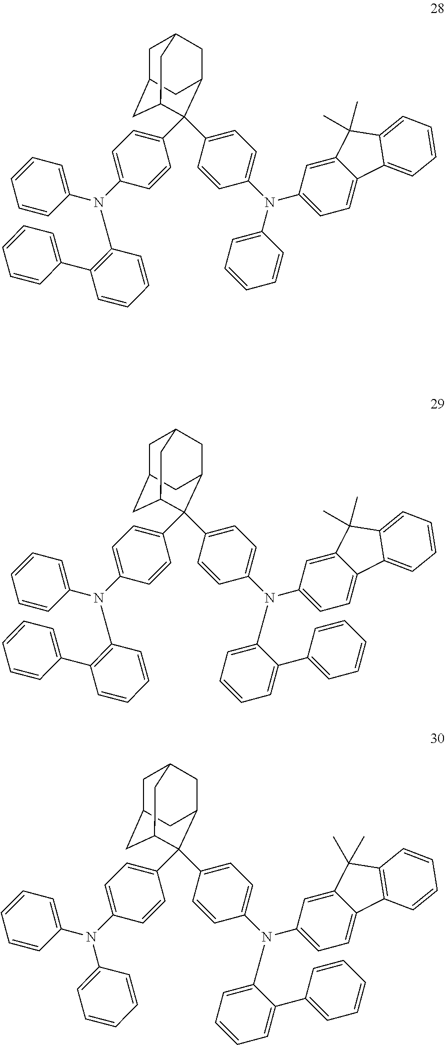 [SMILES: CC1(C)c2ccccc2-c2ccc(N(c3ccccc3)c3ccc(C4(c5ccc(N(c6ccccc6)c6ccccc6-c6ccccc6)cc5)C5CC6CC(C5)CC4C6)cc3)cc21 ; CC1(C)c2ccccc2-c2ccc(N(c3ccc(C4(c5ccc(N(c6ccccc6)c6ccccc6-c6ccccc6)cc5)C5CC6CC(C5)CC4C6)cc3)c3ccccc3-c3ccccc3)cc21 ; CC1(C)c2ccccc2-c2ccc(N(c3ccc(C4(c5ccc(N(c6ccccc6)c6ccccc6)cc5)C5CC6CC(C5)CC4C6)cc3)c3ccccc3-c3ccccc3)cc21]

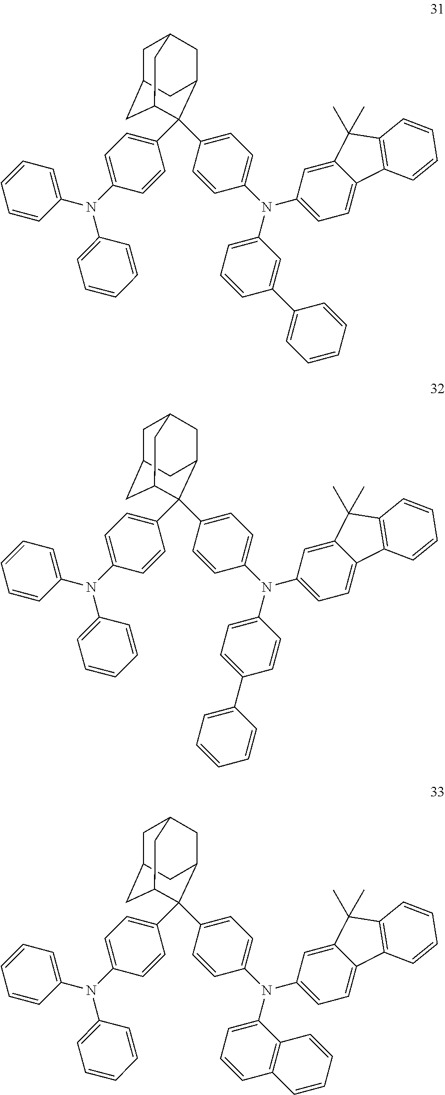 [SMILES: CC1(C)c2ccccc2-c2ccc(N(c3ccc(C4(c5ccc(N(c6ccccc6)c6ccccc6)cc5)C5CC6CC(C5)CC4C6)cc3)c3cccc(-c4ccccc4)c3)cc21 ; CC1(C)c2ccccc2-c2ccc(N(c3ccc(-c4ccccc4)cc3)c3ccc(C4(c5ccc(N(c6ccccc6)c6ccccc6)cc5)C5CC6CC(C5)CC4C6)cc3)cc21 ; CC1(C)c2ccccc2-c2ccc(N(c3ccc(C4(c5ccc(N(c6ccccc6)c6ccccc6)cc5)C5CC6CC(C5)CC4C6)cc3)c3cccc4ccccc34)cc21]

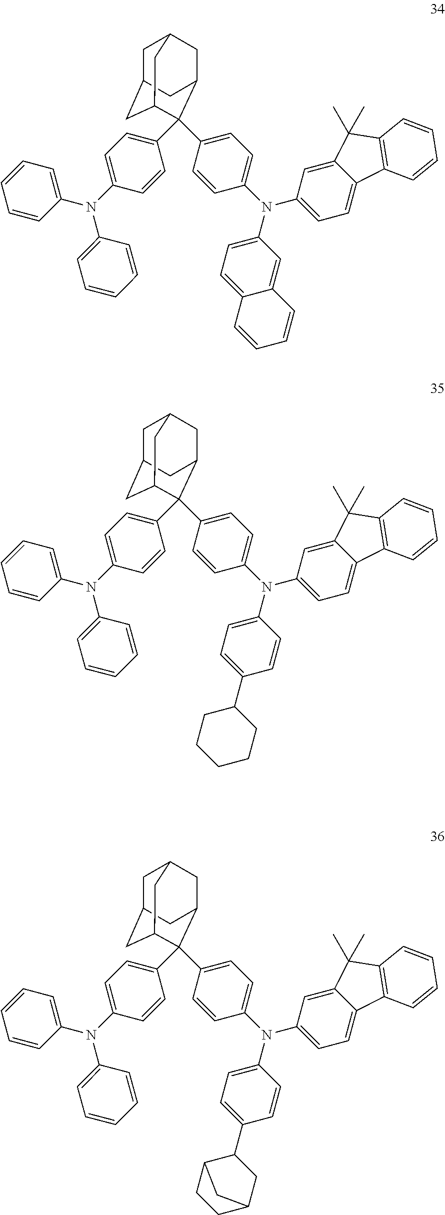 [SMILES: CC1(C)c2ccccc2-c2ccc(N(c3ccc(C4(c5ccc(N(c6ccccc6)c6ccccc6)cc5)C5CC6CC(C5)CC4C6)cc3)c3ccc4ccccc4c3)cc21 ; CC1(C)c2ccccc2-c2ccc(N(c3ccc(C4CCCCC4)cc3)c3ccc(C4(c5ccc(N(c6ccccc6)c6ccccc6)cc5)C5CC6CC(C5)CC4C6)cc3)cc21 ; CC1(C)c2ccccc2-c2ccc(N(c3ccc(C4CC5CCC4C5)cc3)c3ccc(C4(c5ccc(N(c6ccccc6)c6ccccc6)cc5)C5CC6CC(C5)CC4C6)cc3)cc21]

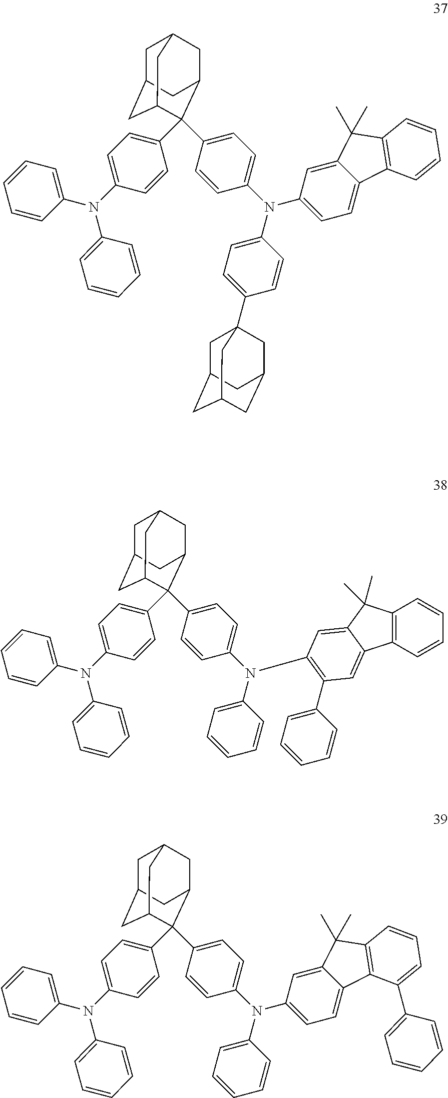 [SMILES: CC1(C)c2ccccc2-c2ccc(N(c3ccc(C45CC6CC(CC(C6)C4)C5)cc3)c3ccc(C4(c5ccc(N(c6ccccc6)c6ccccc6)cc5)C5CC6CC(C5)CC4C6)cc3)cc21 ; CC1(C)c2ccccc2-c2cc(-c3ccccc3)c(N(c3ccccc3)c3ccc(C4(c5ccc(N(c6ccccc6)c6ccccc6)cc5)C5CC6CC(C5)CC4C6)cc3)cc21 ; CC1(C)c2cc(N(c3ccccc3)c3ccc(C4(c5ccc(N(c6ccccc6)c6ccccc6)cc5)C5CC6CC(C5)CC4C6)cc3)ccc2-c2c(-c3ccccc3)cccc21]

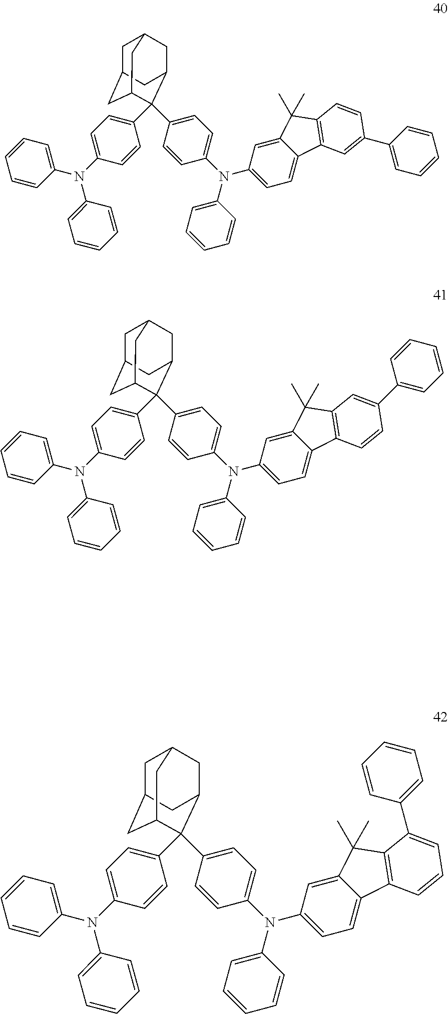 [SMILES: CC1(C)c2ccc(-c3ccccc3)cc2-c2ccc(N(c3ccccc3)c3ccc(C4(c5ccc(N(c6ccccc6)c6ccccc6)cc5)C5CC6CC(C5)CC4C6)cc3)cc21 ; CC1(C)c2cc(-c3ccccc3)ccc2-c2ccc(N(c3ccccc3)c3ccc(C4(c5ccc(N(c6ccccc6)c6ccccc6)cc5)C5CC6CC(C5)CC4C6)cc3)cc21 ; CC1(C)c2cc(N(c3ccccc3)c3ccc(C4(c5ccc(N(c6ccccc6)c6ccccc6)cc5)C5CC6CC(C5)CC4C6)cc3)ccc2-c2cccc(-c3ccccc3)c21]

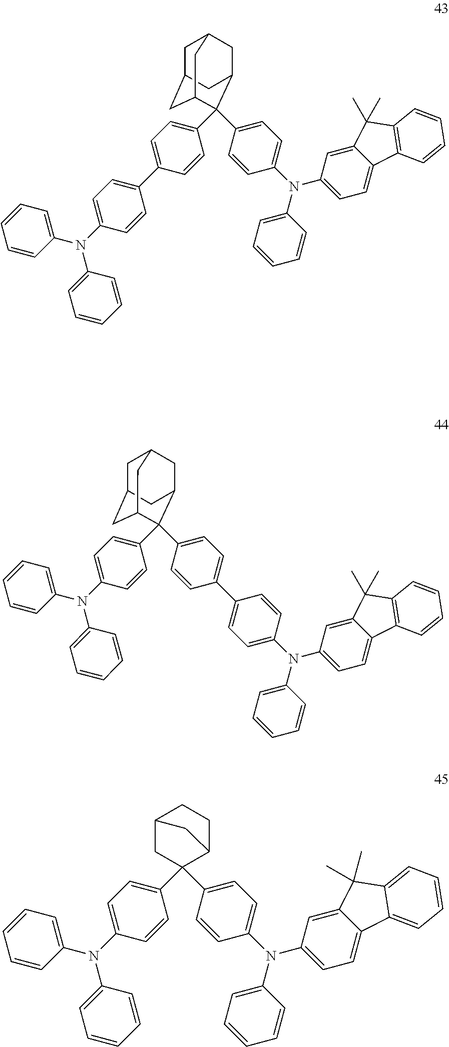 [SMILES: CC1(C)c2ccccc2-c2ccc(N(c3ccccc3)c3ccc(C4(c5ccc(-c6ccc(N(c7ccccc7)c7ccccc7)cc6)cc5)C5CC6CC(C5)CC4C6)cc3)cc21 ; CC1(C)c2ccccc2-c2ccc(N(c3ccccc3)c3ccc(-c4ccc(C5(c6ccc(N(c7ccccc7)c7ccccc7)cc6)C6CC7CC(C6)CC5C7)cc4)cc3)cc21 ; CC1(C)c2ccccc2-c2ccc(N(c3ccccc3)c3ccc(C4(c5ccc(N(c6ccccc6)c6ccccc6)cc5)CC5CCC4C5)cc3)cc21]

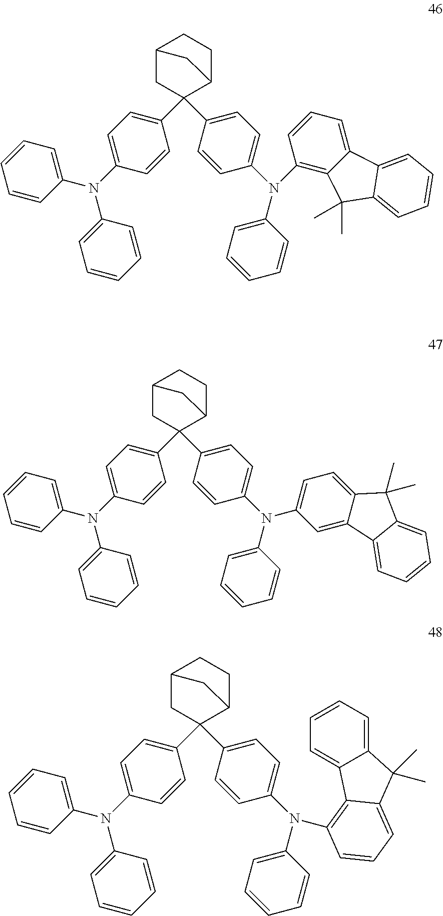 [SMILES: CC1(C)c2ccccc2-c2cccc(N(c3ccccc3)c3ccc(C4(c5ccc(N(c6ccccc6)c6ccccc6)cc5)CC5CCC4C5)cc3)c21 ; CC1(C)c2ccccc2-c2cc(N(c3ccccc3)c3ccc(C4(c5ccc(N(c6ccccc6)c6ccccc6)cc5)CC5CCC4C5)cc3)ccc21 ; CC1(C)c2ccccc2-c2c(N(c3ccccc3)c3ccc(C4(c5ccc(N(c6ccccc6)c6ccccc6)cc5)CC5CCC4C5)cc3)cccc21]

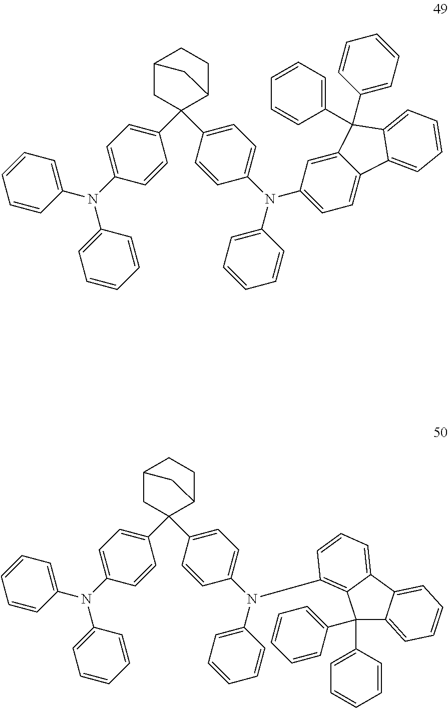 [SMILES: c1ccc(N(c2ccccc2)c2ccc(C3(c4ccc(N(c5ccccc5)c5ccc6c(c5)C(c5ccccc5)(c5ccccc5)c5ccccc5-6)cc4)CC4CCC3C4)cc2)cc1 ; c1ccc(N(c2ccccc2)c2ccc(C3(c4ccc(N(c5ccccc5)c5cccc6c5C(c5ccccc5)(c5ccccc5)c5ccccc5-6)cc4)CC4CCC3C4)cc2)cc1]

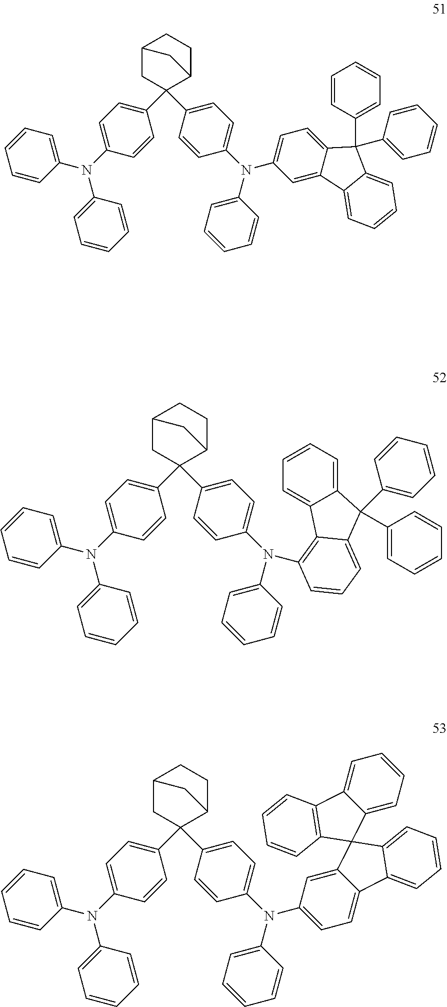 [SMILES: c1ccc(N(c2ccccc2)c2ccc(C3(c4ccc(N(c5ccccc5)c5ccc6c(c5)-c5ccccc5C6(c5ccccc5)c5ccccc5)cc4)CC4CCC3C4)cc2)cc1 ; c1ccc(N(c2ccccc2)c2ccc(C3(c4ccc(N(c5ccccc5)c5cccc6c5-c5ccccc5C6(c5ccccc5)c5ccccc5)cc4)CC4CCC3C4)cc2)cc1 ; c1ccc(N(c2ccccc2)c2ccc(C3(c4ccc(N(c5ccccc5)c5ccc6c(c5)C5(c7ccccc7-c7ccccc75)c5ccccc5-6)cc4)CC4CCC3C4)cc2)cc1]

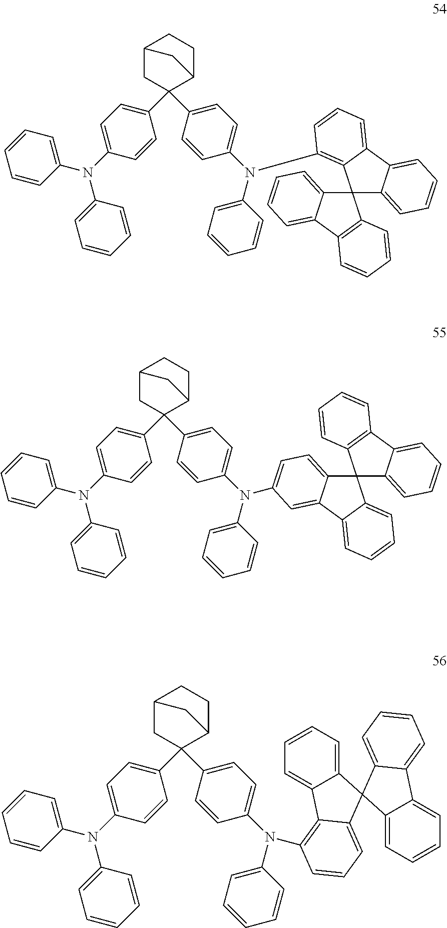 [SMILES: c1ccc(N(c2ccccc2)c2ccc(C3(c4ccc(N(c5ccccc5)c5cccc6c5C5(c7ccccc7-c7ccccc75)c5ccccc5-6)cc4)CC4CCC3C4)cc2)cc1 ; c1ccc(N(c2ccccc2)c2ccc(C3(c4ccc(N(c5ccccc5)c5ccc6c(c5)-c5ccccc5C65c6ccccc6-c6ccccc65)cc4)CC4CCC3C4)cc2)cc1 ; c1ccc(N(c2ccccc2)c2ccc(C3(c4ccc(N(c5ccccc5)c5cccc6c5-c5ccccc5C65c6ccccc6-c6ccccc65)cc4)CC4CCC3C4)cc2)cc1]

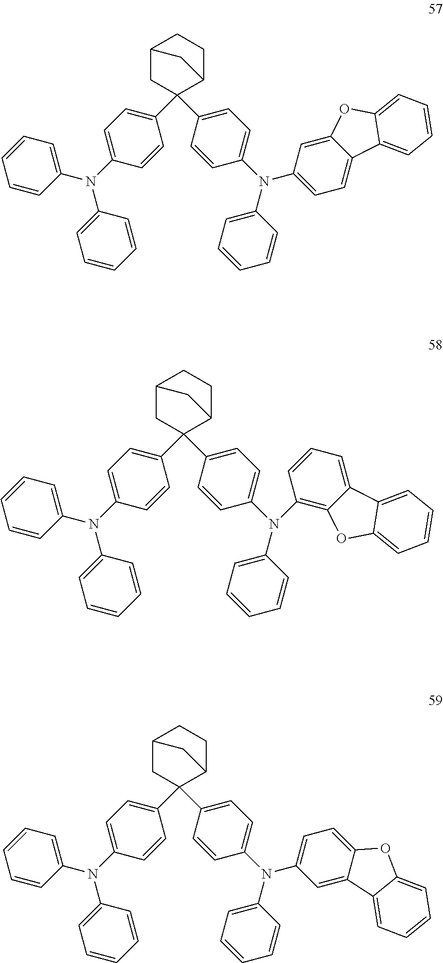 [SMILES: c1ccc(N(c2ccccc2)c2ccc(C3(c4ccc(N(c5ccccc5)c5ccc6c(c5)oc5ccccc56)cc4)CC4CCC3C4)cc2)cc1 ; c1ccc(N(c2ccccc2)c2ccc(C3(c4ccc(N(c5ccccc5)c5cccc6c5oc5ccccc56)cc4)CC4CCC3C4)cc2)cc1 ; c1ccc(N(c2ccccc2)c2ccc(C3(c4ccc(N(c5ccccc5)c5ccc6oc7ccccc7c6c5)cc4)CC4CCC3C4)cc2)cc1]

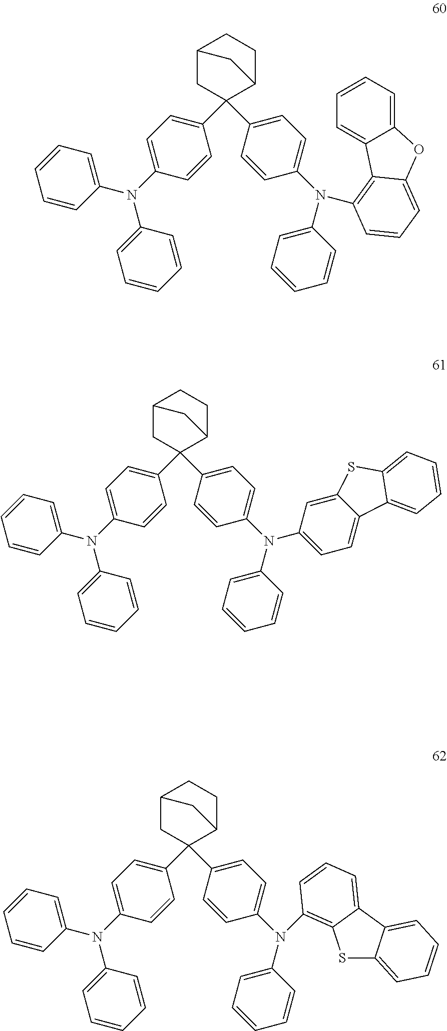 [SMILES: c1ccc(N(c2ccccc2)c2ccc(C3(c4ccc(N(c5ccccc5)c5cccc6oc7ccccc7c56)cc4)CC4CCC3C4)cc2)cc1 ; c1ccc(N(c2ccccc2)c2ccc(C3(c4ccc(N(c5ccccc5)c5ccc6c(c5)sc5ccccc56)cc4)CC4CCC3C4)cc2)cc1 ; c1ccc(N(c2ccccc2)c2ccc(C3(c4ccc(N(c5ccccc5)c5cccc6c5sc5ccccc56)cc4)CC4CCC3C4)cc2)cc1]

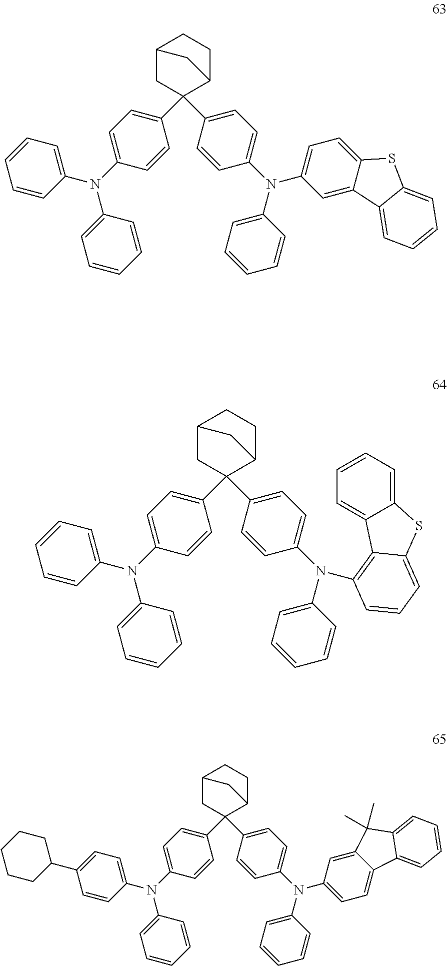 [SMILES: c1ccc(N(c2ccccc2)c2ccc(C3(c4ccc(N(c5ccccc5)c5ccc6sc7ccccc7c6c5)cc4)CC4CCC3C4)cc2)cc1 ; c1ccc(N(c2ccccc2)c2ccc(C3(c4ccc(N(c5ccccc5)c5cccc6sc7ccccc7c56)cc4)CC4CCC3C4)cc2)cc1 ; CC1(C)c2ccccc2-c2ccc(N(c3ccccc3)c3ccc(C4(c5ccc(N(c6ccccc6)c6ccc(C7CCCCC7)cc6)cc5)CC5CCC4C5)cc3)cc21]

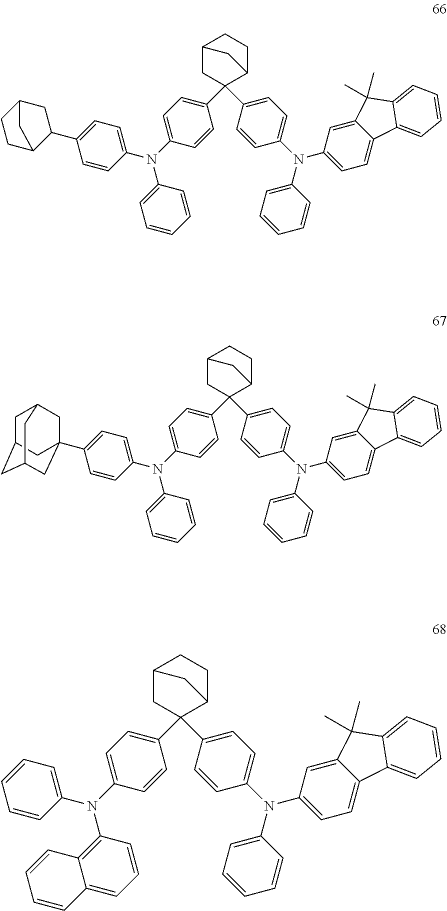 [SMILES: CC1(C)c2ccccc2-c2ccc(N(c3ccccc3)c3ccc(C4(c5ccc(N(c6ccccc6)c6ccc(C7CC8CCC7C8)cc6)cc5)CC5CCC4C5)cc3)cc21 ; CC1(C)c2ccccc2-c2ccc(N(c3ccccc3)c3ccc(C4(c5ccc(N(c6ccccc6)c6ccc(C78CC9CC(CC(C9)C7)C8)cc6)cc5)CC5CCC4C5)cc3)cc21 ; CC1(C)c2ccccc2-c2ccc(N(c3ccccc3)c3ccc(C4(c5ccc(N(c6ccccc6)c6cccc7ccccc67)cc5)CC5CCC4C5)cc3)cc21]

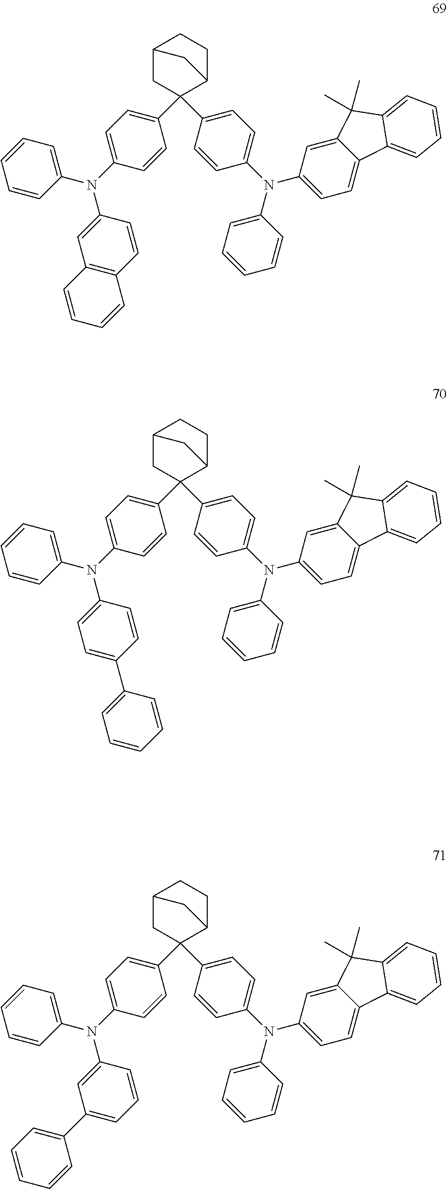 [SMILES: CC1(C)c2ccccc2-c2ccc(N(c3ccccc3)c3ccc(C4(c5ccc(N(c6ccccc6)c6ccc7ccccc7c6)cc5)CC5CCC4C5)cc3)cc21 ; CC1(C)c2ccccc2-c2ccc(N(c3ccccc3)c3ccc(C4(c5ccc(N(c6ccccc6)c6ccc(-c7ccccc7)cc6)cc5)CC5CCC4C5)cc3)cc21 ; CC1(C)c2ccccc2-c2ccc(N(c3ccccc3)c3ccc(C4(c5ccc(N(c6ccccc6)c6cccc(-c7ccccc7)c6)cc5)CC5CCC4C5)cc3)cc21]

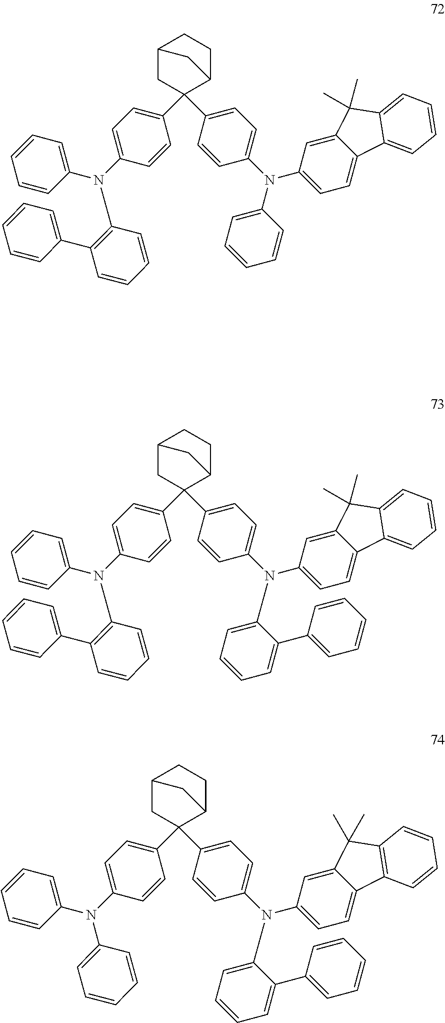 [SMILES: CC1(C)c2ccccc2-c2ccc(N(c3ccccc3)c3ccc(C4(c5ccc(N(c6ccccc6)c6ccccc6-c6ccccc6)cc5)CC5CCC4C5)cc3)cc21 ; CC1(C)c2ccccc2-c2ccc(N(c3ccc(C4(c5ccc(N(c6ccccc6)c6ccccc6-c6ccccc6)cc5)CC5CCC4C5)cc3)c3ccccc3-c3ccccc3)cc21 ; CC1(C)c2ccccc2-c2ccc(N(c3ccc(C4(c5ccc(N(c6ccccc6)c6ccccc6)cc5)CC5CCC4C5)cc3)c3ccccc3-c3ccccc3)cc21]

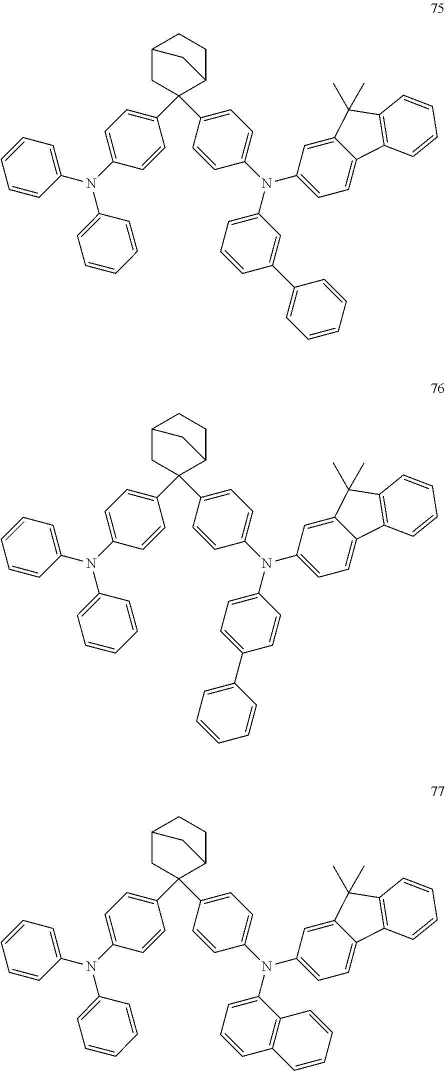 [SMILES: CC1(C)c2ccccc2-c2ccc(N(c3ccc(C4(c5ccc(N(c6ccccc6)c6ccccc6)cc5)CC5CCC4C5)cc3)c3cccc(-c4ccccc4)c3)cc21 ; CC1(C)c2ccccc2-c2ccc(N(c3ccc(-c4ccccc4)cc3)c3ccc(C4(c5ccc(N(c6ccccc6)c6ccccc6)cc5)CC5CCC4C5)cc3)cc21 ; CC1(C)c2ccccc2-c2ccc(N(c3ccc(C4(c5ccc(N(c6ccccc6)c6ccccc6)cc5)CC5CCC4C5)cc3)c3cccc4ccccc34)cc21]

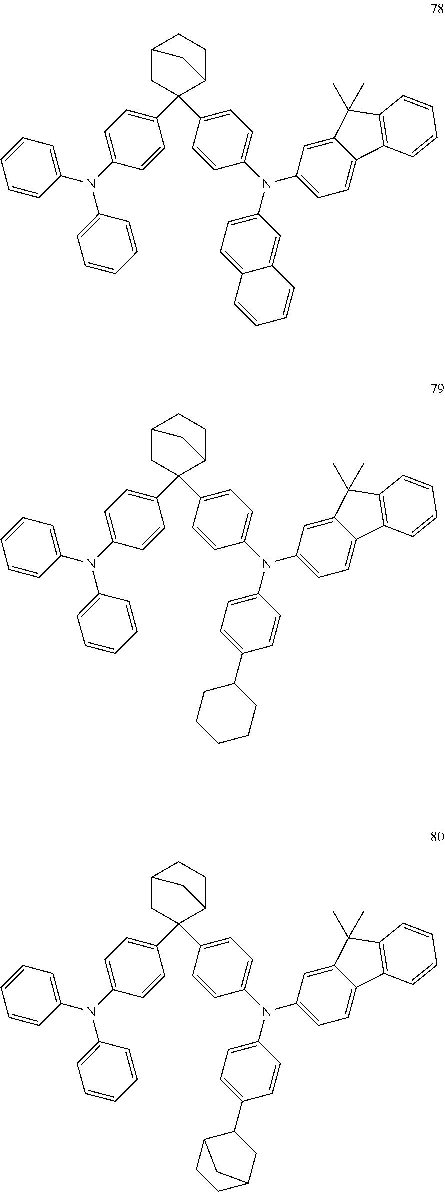 [SMILES: CC1(C)c2ccccc2-c2ccc(N(c3ccc(C4(c5ccc(N(c6ccccc6)c6ccccc6)cc5)CC5CCC4C5)cc3)c3ccc4ccccc4c3)cc21 ; CC1(C)c2ccccc2-c2ccc(N(c3ccc(C4CCCCC4)cc3)c3ccc(C4(c5ccc(N(c6ccccc6)c6ccccc6)cc5)CC5CCC4C5)cc3)cc21 ; CC1(C)c2ccccc2-c2ccc(N(c3ccc(C4CC5CCC4C5)cc3)c3ccc(C4(c5ccc(N(c6ccccc6)c6ccccc6)cc5)CC5CCC4C5)cc3)cc21]

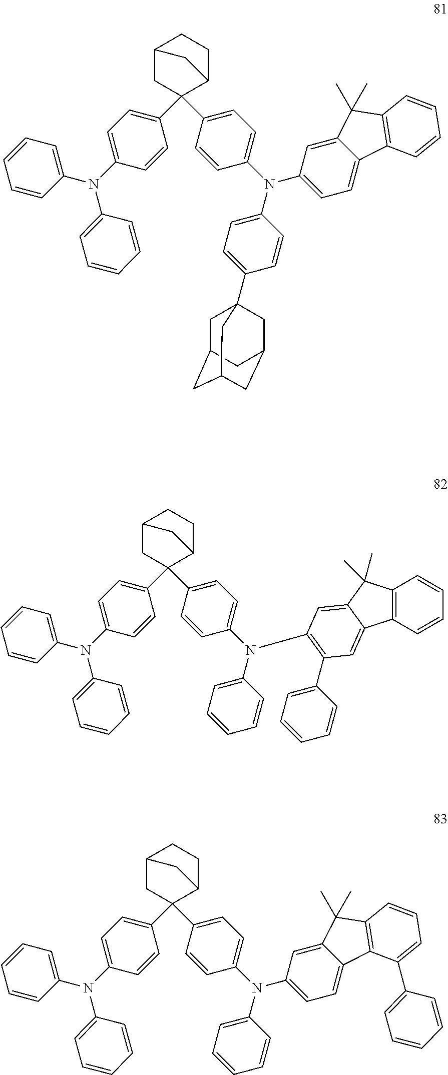 [SMILES: CC1(C)c2ccccc2-c2ccc(N(c3ccc(C45CC6CC(CC(C6)C4)C5)cc3)c3ccc(C4(c5ccc(N(c6ccccc6)c6ccccc6)cc5)CC5CCC4C5)cc3)cc21 ; CC1(C)c2ccccc2-c2cc(-c3ccccc3)c(N(c3ccccc3)c3ccc(C4(c5ccc(N(c6ccccc6)c6ccccc6)cc5)CC5CCC4C5)cc3)cc21 ; CC1(C)c2cc(N(c3ccccc3)c3ccc(C4(c5ccc(N(c6ccccc6)c6ccccc6)cc5)CC5CCC4C5)cc3)ccc2-c2c(-c3ccccc3)cccc21]

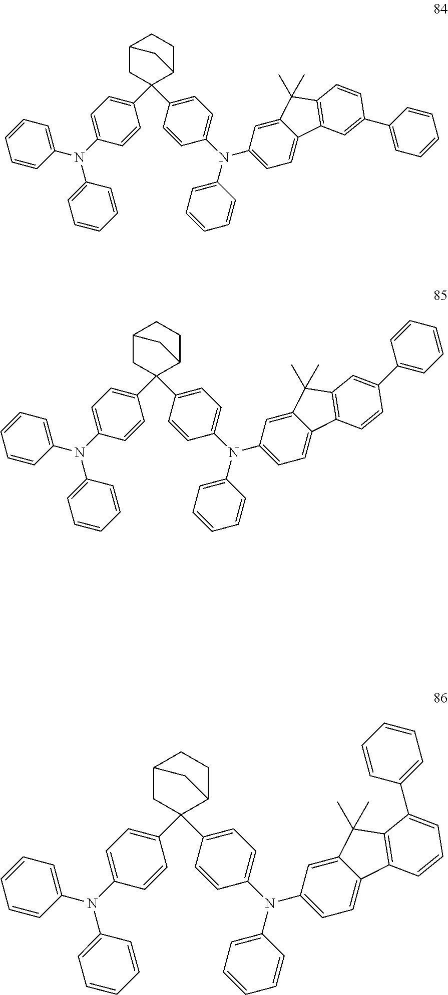 [SMILES: CC1(C)c2ccc(-c3ccccc3)cc2-c2ccc(N(c3ccccc3)c3ccc(C4(c5ccc(N(c6ccccc6)c6ccccc6)cc5)CC5CCC4C5)cc3)cc21 ; CC1(C)c2cc(-c3ccccc3)ccc2-c2ccc(N(c3ccccc3)c3ccc(C4(c5ccc(N(c6ccccc6)c6ccccc6)cc5)CC5CCC4C5)cc3)cc21 ; CC1(C)c2cc(N(c3ccccc3)c3ccc(C4(c5ccc(N(c6ccccc6)c6ccccc6)cc5)CC5CCC4C5)cc3)ccc2-c2cccc(-c3ccccc3)c21]

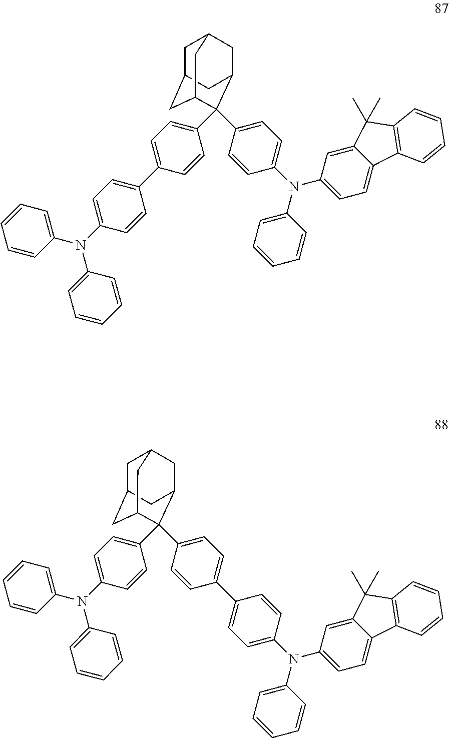 [SMILES: CC1(C)c2ccccc2-c2ccc(N(c3ccccc3)c3ccc(C4(c5ccc(-c6ccc(N(c7ccccc7)c7ccccc7)cc6)cc5)C5CC6CC(C5)CC4C6)cc3)cc21 ; CC1(C)c2ccccc2-c2ccc(N(c3ccccc3)c3ccc(-c4ccc(C5(c6ccc(N(c7ccccc7)c7ccccc7)cc6)C6CC7CC(C6)CC5C7)cc4)cc3)cc21]

In an embodiment, additionally provided is a diamine-based compound including a condensed polycyclic group and an aromatic substituted amine group,

the aromatic substituted amine group may include a fluorene group unsubstituted or substituted with at least one R₁₀ₐ or a fluorene derivative group unsubstituted or substituted with at least one R₁₀ₐ,

an energy level of a lowest unoccupied molecular orbital (LUMO) may be from about −5.3 eV to about −5.1 eV,

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any suitable combinations thereof;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any suitable combinations thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

Q₁, Q₂, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any suitable combinations thereof, and

Q₁ and Q₂ may optionally be bonded together to form a C₃-C₆₀ carbocyclic group unsubstituted or substituted with at least one R₁₀ₐ or a C₁-C₆₀ heterocyclic group unsubstituted or substituted with at least one R₁₀ₐ.

In an embodiment, the condensed polycyclic group may be one of an adamantanyl group, a norbornanyl group, a norbornenyl group, a bicyclo[1.1.1]pentyl group, a bicyclo[2.1.1]hexyl group, a bicyclo[2.2.2]octyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.2]octyl group, a mono(C₁-C₂₀ alkyl)adamantanyl group, a di(C₁-C₂₀ alkyl)adamantanyl group, a mono(C₁-C₂₀ alkyl)norbornanyl group, a di(C₁-C₂₀ alkyl)norbornanyl group, a mono(C₁-C₂₀ alkyl)norbornenyl group, or a di(C₁-C₂₀ alkyl)norbornenyl group.

In an embodiment, the fluorene derivative group may include at least one of a carbazole moiety, a dibenzosilole moiety, a dibenzothiophene moiety, or a dibenzofuran moiety.

In an embodiment, the condensed polycyclic group and the amine group may be linked together through at least one aromatic linking group, the aromatic linking group may be a divalent linking group of a C₆-C₆₀ aryl group unsubstituted or substituted with at least one R₁₀ₐ or a divalent linking group of a C₃-C₆₀ heteroaryl group unsubstituted or substituted with at least one R₁₀ₐ, and

R₁₀ₐ is the same as described in the present disclosure.

The diamine-based compound represented by Formula 1 includes a linking group represented by A and has a molecular structure having large steric hindrance. Furthermore, when the diamine-based compound represented by Formula 1 includes at least one substituent represented by Formula 1-2, the molecular structure has a greater steric hindrance (than the steric hindrance observed if the at least one substituent represented by Formula 1-2 were not included). Accordingly, the diamine-based compound may maintain an optimal intermolecular density.

Also, a heteroatom and an unshared electron pair of the heteroatom in Formula 1-2 are located on the outer side of the diamine-based compound represented by Formula 1 and are relatively less covered, and excellent hole mobility may be achieved through an increase in interaction by the unshared electron pair.

Furthermore, by varying Rₐ and R_{b} in Formula 1 or varying Ar₂ and Ar₃ in Formula 1-1, an energy level of a highest occupied molecular orbital (HOMO), an energy level of a LUMO, and/or an energy level of T₁, a refractive index of the diamine-based compound represented by Formula 1 may be finely adjusted.

As a result, hole mobility and resonance effect may be improved (e.g., may each be improved or may each be similarly improved), and an electronic device, for example, an organic light-emitting device, including the diamine-based compound may have a low driving voltage, a high efficiency, and a long lifespan.

Methods of synthesizing the diamine-based compound represented by Formula 1 may be easily understood to those of ordinary skill in the art by referring to Synthesis Examples and Examples described herein.

At least one diamine-based compound represented by Formula 1 may be used in a light-emitting device (for example, an organic light-emitting device). Thus, provided is a light-emitting device including a first electrode, a second electrode facing the first electrode, an interlayer arranged between the first electrode and the second electrode and including an emission layer, and the diamine-based compound represented by Formula 1 as described in the present disclosure.

In an embodiment,

the first electrode of the light-emitting device may be an anode,

the second electrode of the light-emitting device may be a cathode,

the interlayer may further include a hole transport region between the first electrode and the emission layer and an electron transport region between the emission layer and the second electrode,

the hole transport region may include a hole injection layer, a hole transport layer, an emission auxiliary layer, an electron blocking layer, or any suitable combination thereof, and

the electron transport region may include a buffer layer, a hole blocking layer, an electron control layer, an electron transport layer, an electron injection layer, or any suitable combination thereof.

In an embodiment, the diamine-based compound may be included between the first electrode and the second electrode of the light-emitting device. Therefore, the diamine-based compound may be included in the interlayer of the light-emitting device, for example, in the emission layer of the interlayer.

In an embodiment, the emission layer of the interlayer of the light-emitting device may include a dopant and a host, and the diamine-based compound may be included in the host. For example, the diamine-based compound may serve as a host. The emission layer may emit red light, green light, blue light, and/or white light. For example, the emission layer may emit blue light. The blue light may have a maximum emission wavelength in a range of about 40 nm to about 490 nm.

In an embodiment, the emission layer of the interlayer of the light-emitting device may include a dopant and a host, the diamine-based compound may be included in the host, and the dopant may emit blue light. For example, the dopant may include a transition metal and ligand(s) in the number of m, m may be an integer from 1 to 6, the ligand(s) in the number of m may be identical to or different from each other, at least one of the ligand(s) in the number of m may be bound to the transition metal via a carbon-transition metal bond, and the carbon-transition metal bond may be a coordinate bond. For example, at least one of the ligand(s) in the number of m may be a carbene ligand (e.g., Ir(pmp)₃ or the like). The transition metal may be, for example, iridium, platinum, osmium, palladium, rhodium, or gold. More details on the emission layer and the dopant may respectively be the same as those described in the present disclosure.

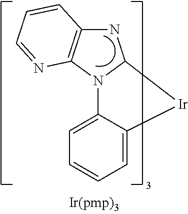

In an embodiment, the light-emitting device may include a capping layer located outside the first electrode or located outside the second electrode.

For example, the light-emitting device may further include at least one of a first capping layer located outside the first electrode and a second capping layer located outside the second electrode, and at least one of the first capping layer and the second capping layer may include the diamine-based compound represented by Formula 1. More details on the first capping layer and/or the second capping layer may respectively be the same as those described in the present disclosure.

In an embodiment, the light-emitting device may include:

a first capping layer arranged outside the first electrode and including the diamine-based compound represented by Formula 1;

a second capping layer arranged outside the second electrode and including the diamine-based compound represented by Formula 1; or

the first capping layer and the second capping layer.

The wording “(interlayer and/or capping layer) includes a diamine-based compound” as used herein may be understood as “(interlayer and/or capping layer) may include one kind of diamine-based compound represented by Formula 1 or two different kinds of diamine-based compounds, each represented by Formula 1”.

For example, the interlayer and/or the capping layer may include Compound 1 only as the diamine-based compound. In this embodiment, Compound 1 may be included in the emission layer of the light-emitting device. In another embodiment, the interlayer may include Compound 1 and Compound 2 as the diamine-based compounds. In this embodiment, Compound 1 and Compound 2 may be present in the same layer (for example, both Compound 1 and Compound 2 may be present in an emission layer), or may be present in different layers (for example, Compound 1 may be present in an emission layer, and Compound 2 may be present in an electron transport region).

The term “interlayer” as used herein refers to a single layer and/or all of a plurality of layers arranged between the first electrode and the second electrode of the light-emitting device.

According to one or more embodiments, an electronic apparatus including the light-emitting device is provided. The electronic apparatus may further include a thin-film transistor. For example, the electronic apparatus may further include a thin-film transistor including a source electrode and a drain electrode, and the first electrode of the light-emitting device may be electrically connected to the source electrode or the drain electrode. In an embodiment, the electronic apparatus may further include a color filter, a color conversion layer, a touch screen layer, a polarizing layer, or any suitable combination thereof. More details on the electronic apparatus are as described in the present disclosure.

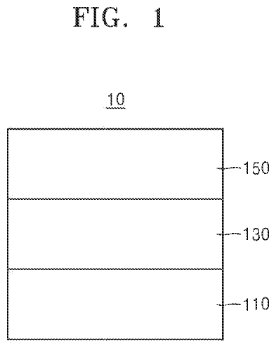

### Description of FIG. 1

FIG. 1 is a schematic cross-sectional view of a light-emitting device 10 according to an embodiment of the disclosure. The light-emitting device 10 includes a first electrode 110, an interlayer 130, and a second electrode 150.

Hereinafter, a structure of the light-emitting device 10 according to an embodiment and a method of manufacturing the light-emitting device 10 will be described in connection with FIG. 1.

### First Electrode 110

In FIG. 1, a substrate may be additionally located under the first electrode 110 or above the second electrode 150. As the substrate, a glass substrate or a plastic substrate may be used. In an embodiment, the substrate may be a flexible substrate, and may include plastics having excellent heat resistance and durability, such as polyimide, polyethylene terephthalate (PET), polycarbonate, polyethylene naphthalate, polyarylate (PAR), polyetherimide, or any suitable combinations thereof.

The first electrode 110 may be formed by, for example, depositing or sputtering a material for forming the first electrode 110 on the substrate. When the first electrode 110 is an anode, a material for forming the first electrode 110 may be a high work function material that facilitates injection of holes.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any suitable combinations thereof. In an embodiment, when the first electrode 110 is a semi-transmissive electrode or a reflective electrode, magnesium (Mg), silver (Ag), aluminum (AI), aluminum-lithium (Al—Li), calcium (Ca), magnesium-indium (Mg—In), magnesium-silver (Mg—Ag), or any suitable combinations thereof may be used as a material for forming a first electrode.

The first electrode 110 may have a single-layered structure consisting of a single layer or a multi-layered structure including a plurality of layers. For example, the first electrode 110 may have a three-layered structure of ITO/Ag/ITO.

### Interlayer 130

The interlayer 130 may be located on the first electrode 110. The interlayer 130 may include an emission layer.

The interlayer 130 may further include a hole transport region between the first electrode 110 and the emission layer and an electron transport region between the emission layer and the second electrode 150.

The interlayer 130 may further include metal-containing compounds such as organometallic compounds, inorganic materials such as quantum dots, and/or the like, in addition to various suitable organic materials.

In an embodiment, the interlayer 130 may include, i) two or more emitting units sequentially stacked between the first electrode 110 and the second electrode 150, and ii) a charge generation layer located between the two emitting units. When the interlayer 130 includes the emitting units and the charge generation layer as described above, the light-emitting device 10 may be a tandem light-emitting device. Hole transport region in interlayer 130

The hole transport region may have: i) a single-layered structure including a single layer consisting of a single material, ii) a single-layered structure including a single layer including a plurality of different materials, or iii) a multi-layered structure including a plurality of layers including different materials.

The hole transport region may include a hole injection layer, a hole transport layer, an emission auxiliary layer, an electron blocking layer, or any suitable combination thereof.

For example, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein, in each structure, layers are stacked sequentially stacked from the first electrode 110.

The hole transport region may include a compound represented by Formula 201, a compound represented by Formula 202, or any suitable combination thereof:

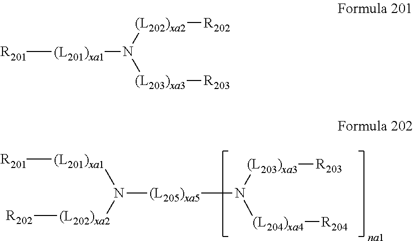

wherein, in Formulae 201 and 202,

L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,

xa1 to xa4 may each independently be an integer from 0 to 5,

xa5 may be an integer from 1 to 10,

R₂₀₁ to R₂₀₄ and 0201 may each independently a C₃-C₆₀ carbocyclic group unsubstituted or substituted with at least one R₁₀ₐ or a C₁-C₆₀ heterocyclic group unsubstituted or substituted with at least one R₁₀ₐ,

R₂₀₁ and R₂₀₂ may optionally be bonded to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group or the like) unsubstituted or substituted with at least one R₁₀ₐ (for example, Compound HT16),

R₂₀₃ and R₂₀₄ may optionally be bonded to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and

na1 may be an integer from 1 to 4.

For example, each of Formulae 201 and 202 may include at least one of groups represented by Formulae CY201 to CY217.

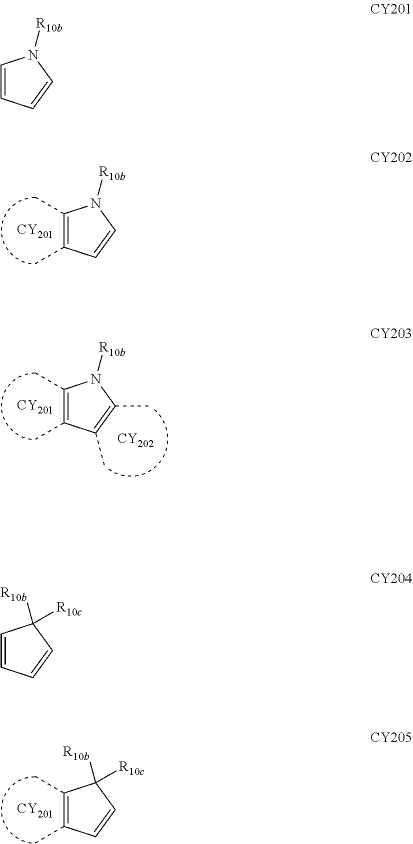

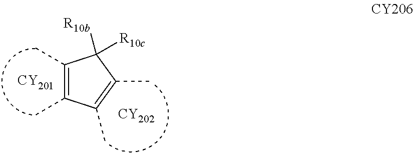

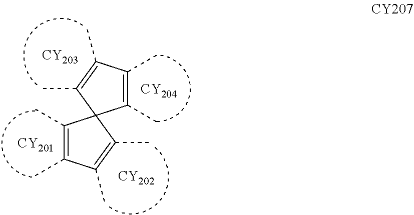

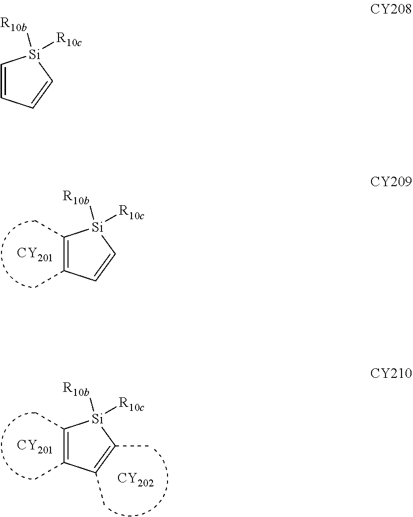

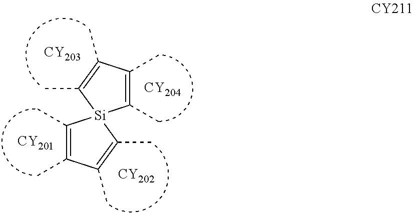

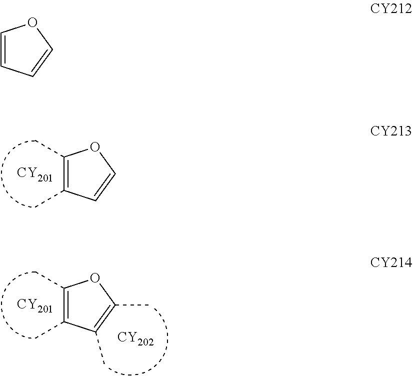

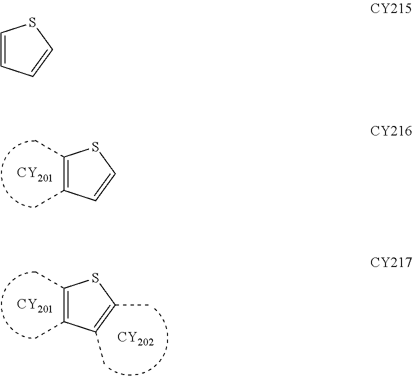

In Formulae CY201 to CY217, R_{10b} and R_{10c} are respectively the same as those described in connection with R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described in the present specification.

In an embodiment, in Formulae CY201 to CY217, ring CY₂₀₁ to ring CY₂₀₄ may each independently be a benzene group, a naphthalene group, a phenanthrene group, or an anthracene group.

In an embodiment, each of Formulae 201 and 202 may include at least one of the groups represented by Formulae CY201 to CY203.

In an embodiment, Formula 201 may include at least one of the groups represented by Formulae CY201 to CY203 and at least one of the groups represented by Formulae CY204 to CY217.

In an embodiment, xa1 in Formula 201 may be 1, R₂₀₁ may be a group represented by one of Formulae CY201 to CY203, xa2 may be 0, and R₂₀₂ may be a group represented by one of Formulae CY204 to CY207.

In an embodiment, each of Formulae 201 and 202 may not include groups represented by Formulae CY201 to CY203.

In an embodiment, each of Formulae 201 and 202 may not include groups represented by Formulae CY201 to CY203, and may include at least one of groups represented by Formulae CY204 to CY217.

In an embodiment, each of Formulae 201 and 202 may not include groups represented by Formulae CY201 to CY217.

For example, the hole transport region may include one of Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any suitable combinations thereof:

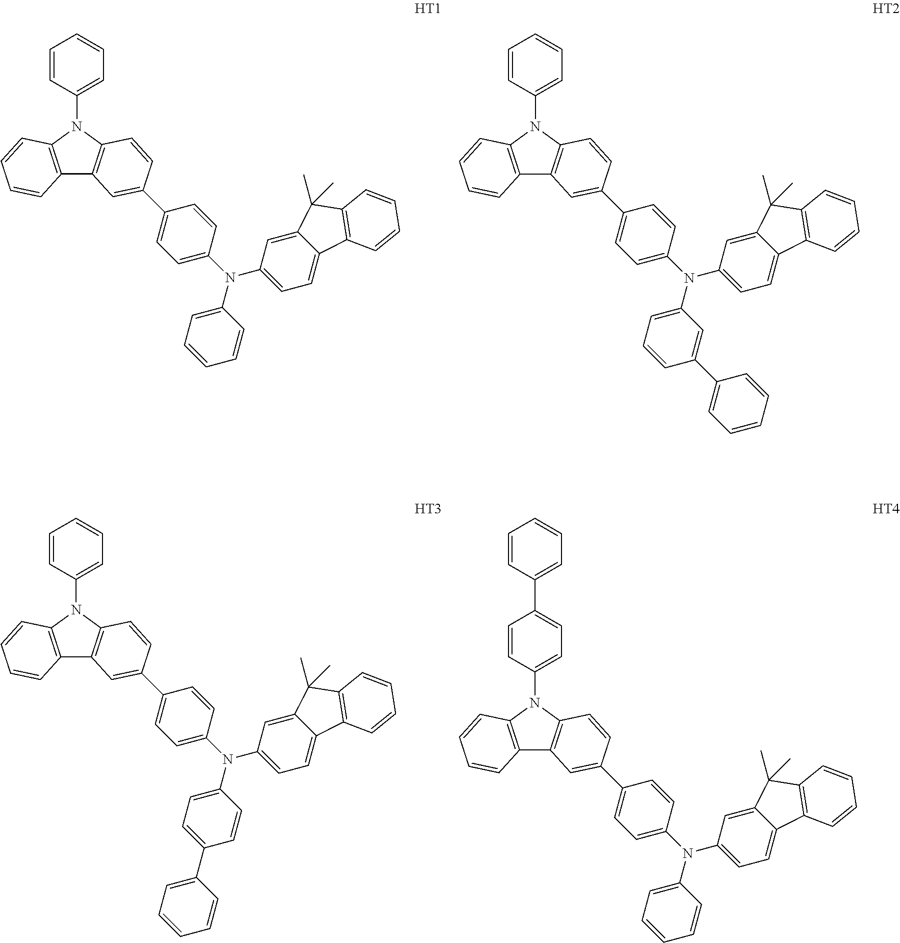 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

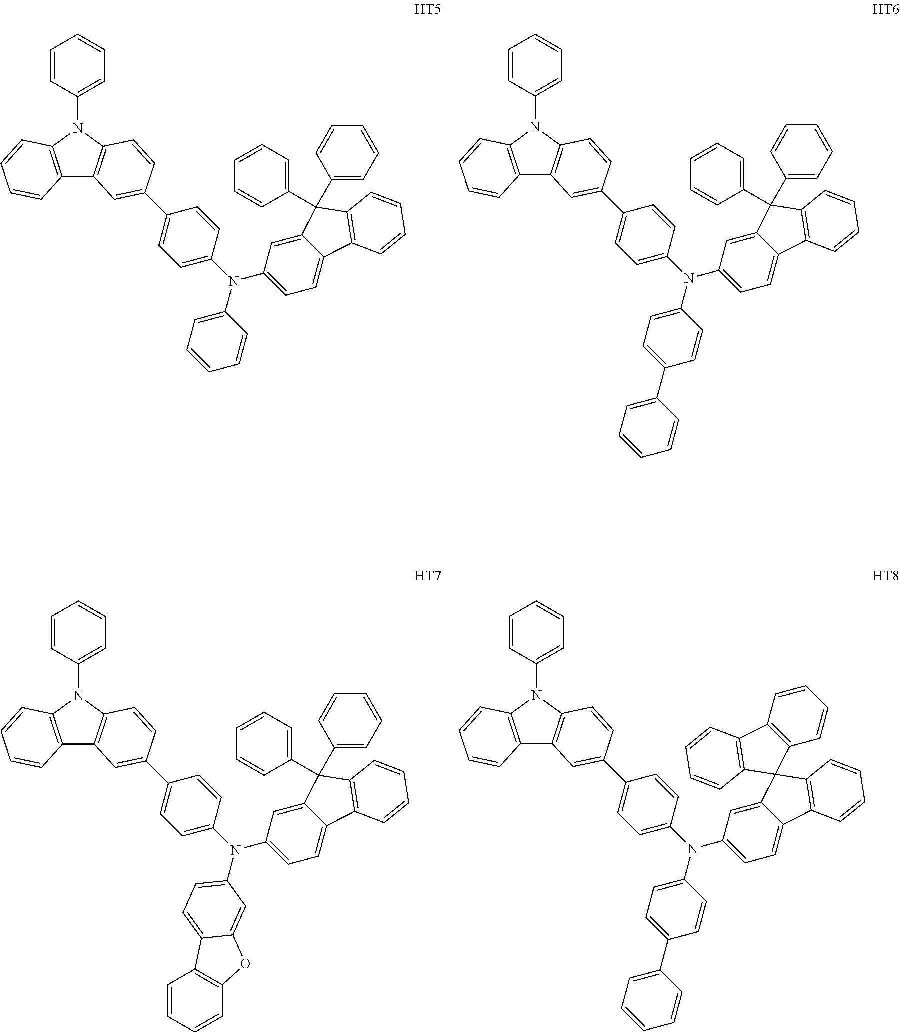 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

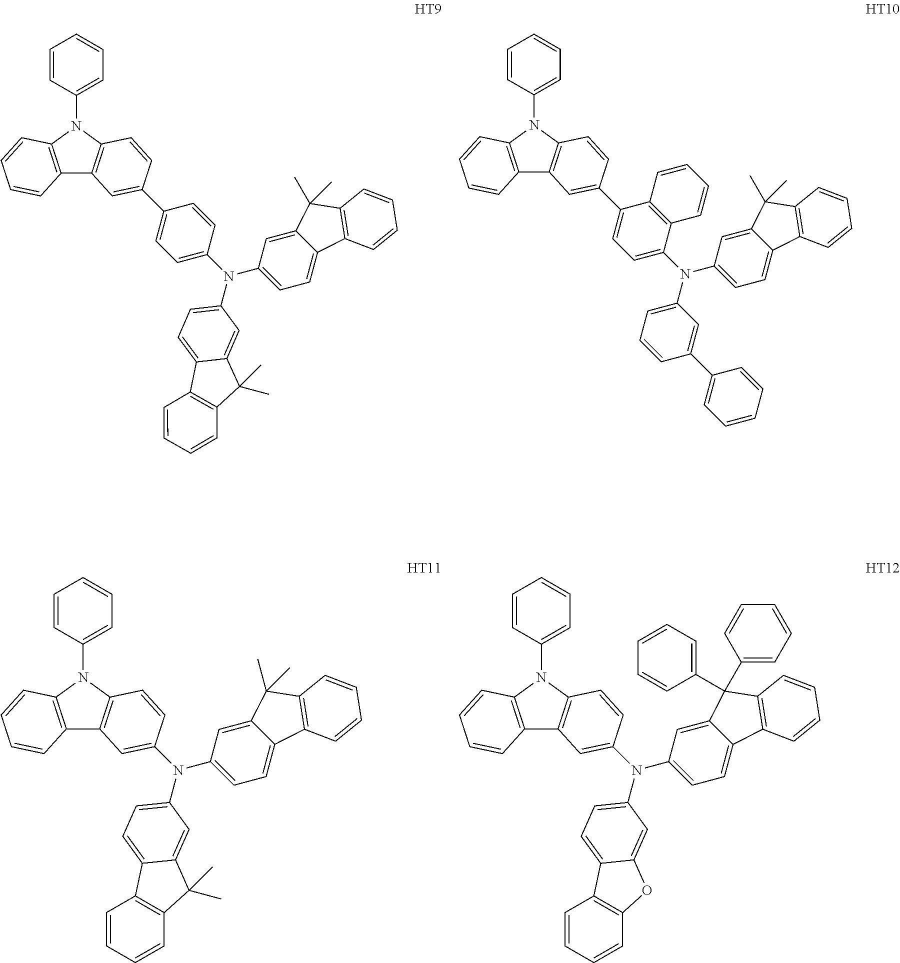 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

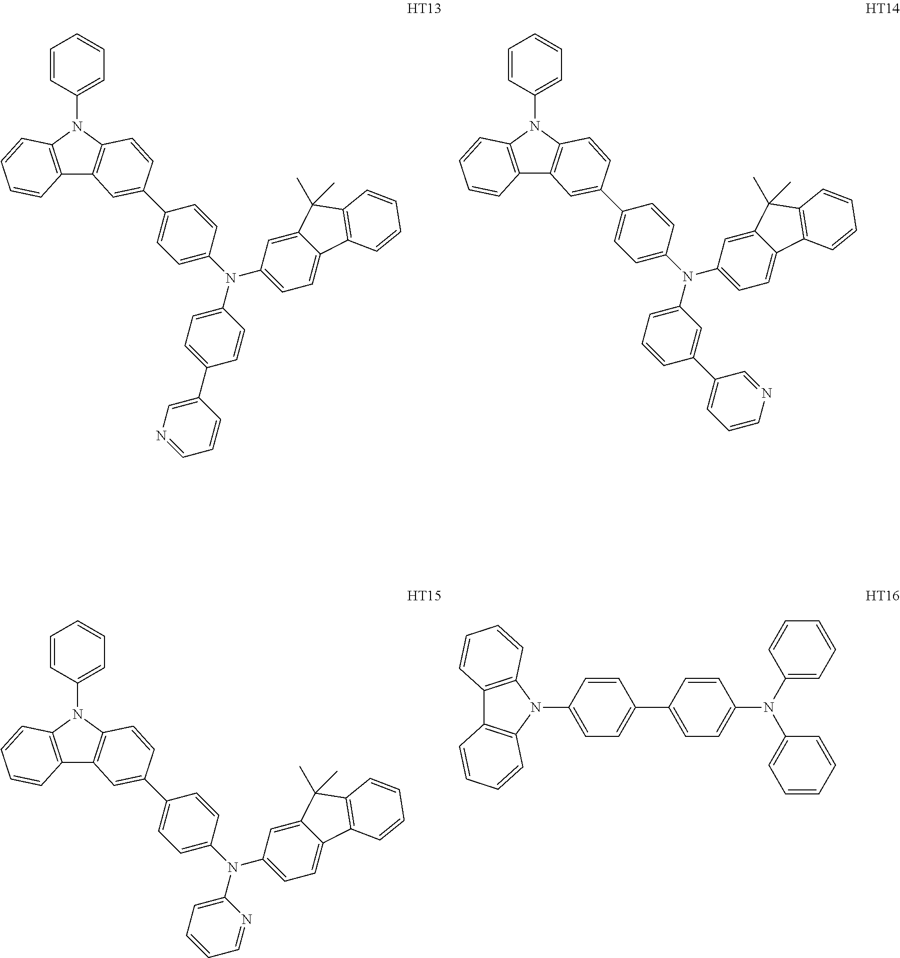 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

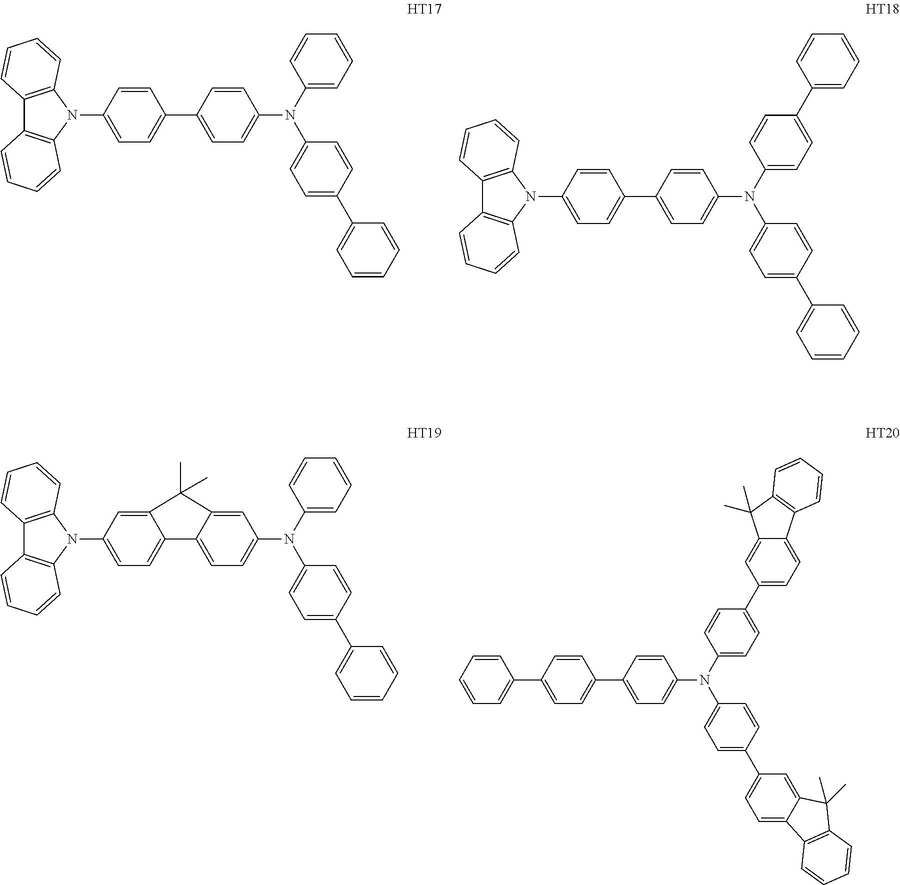 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

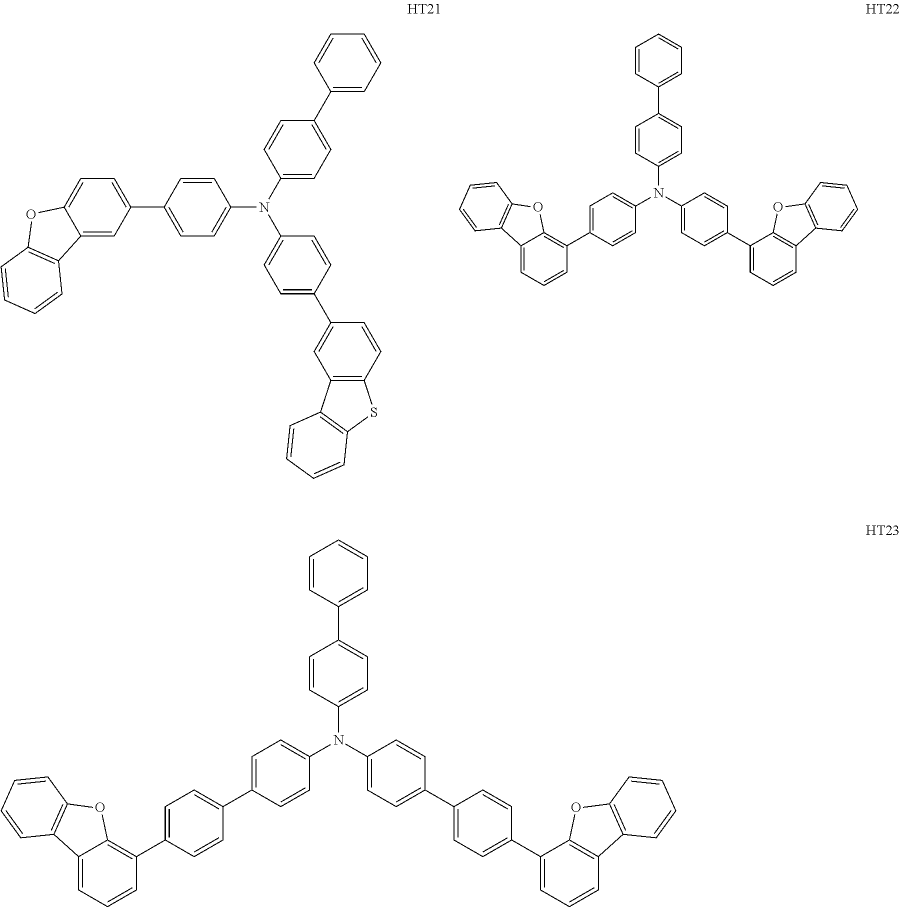 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

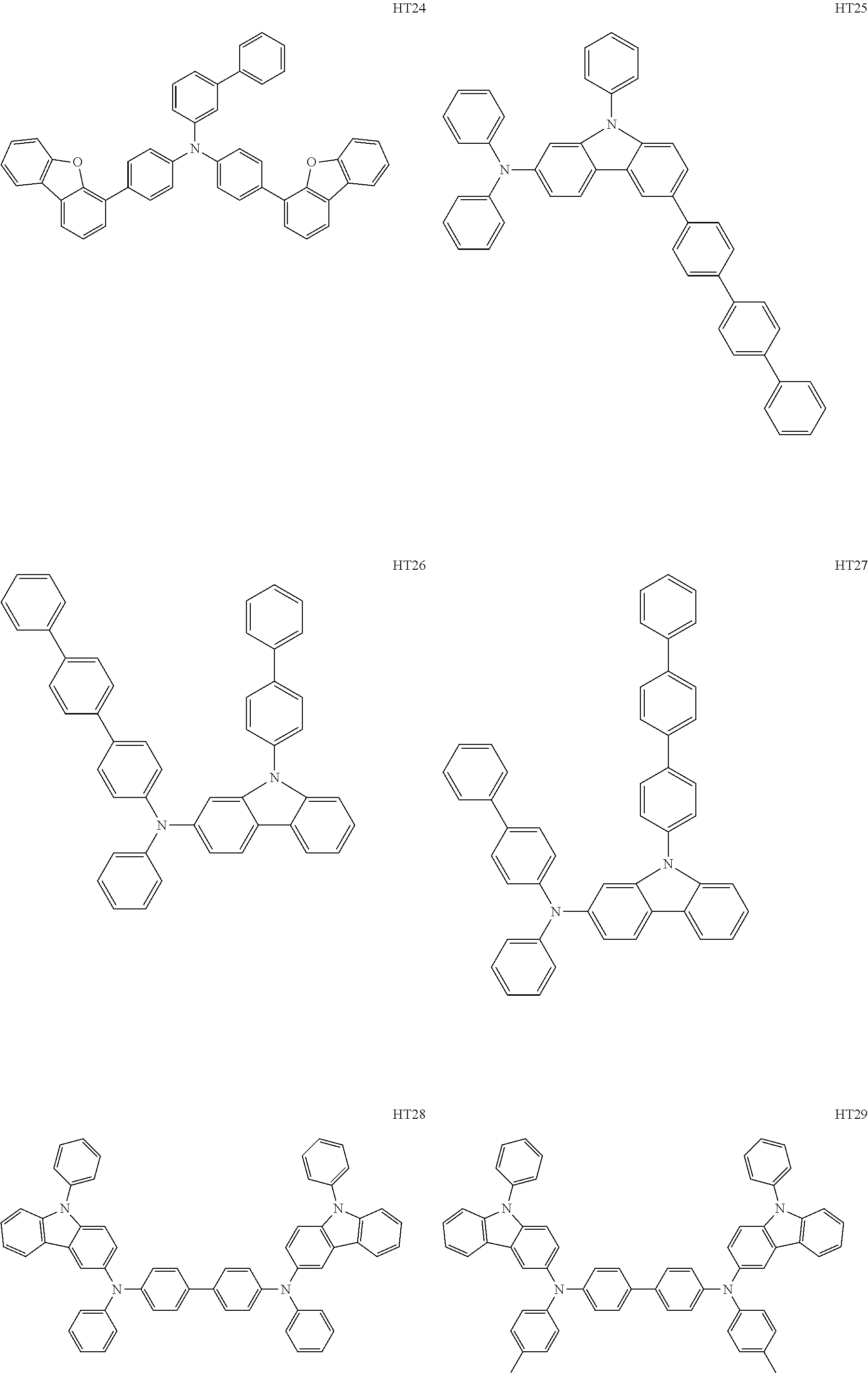

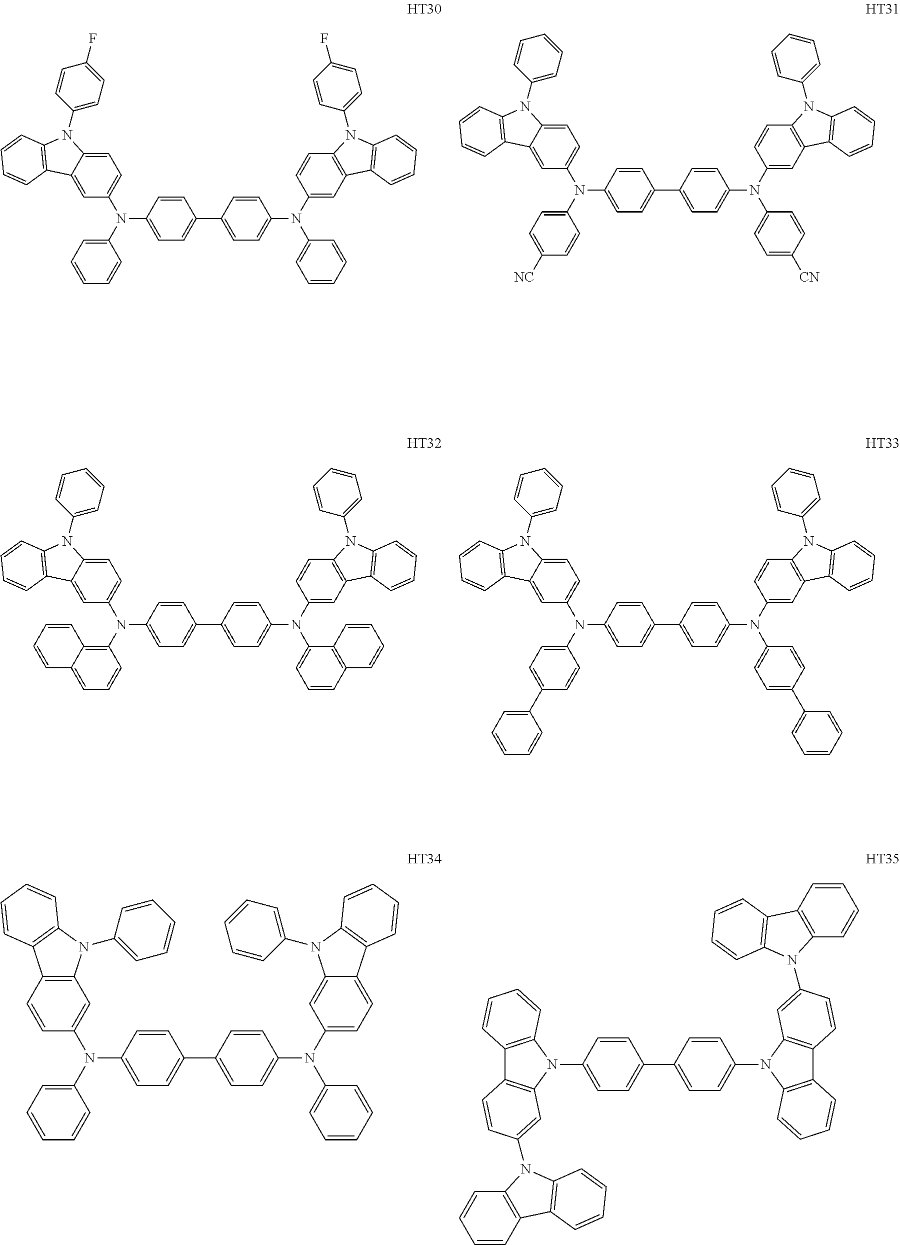

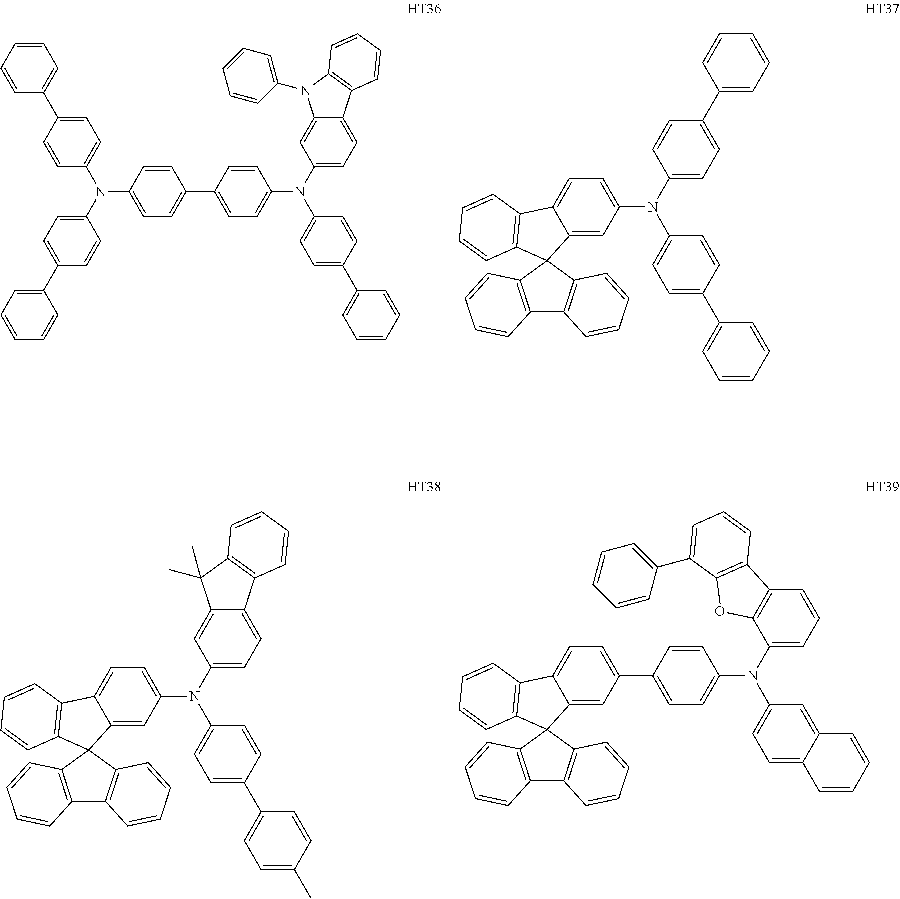 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

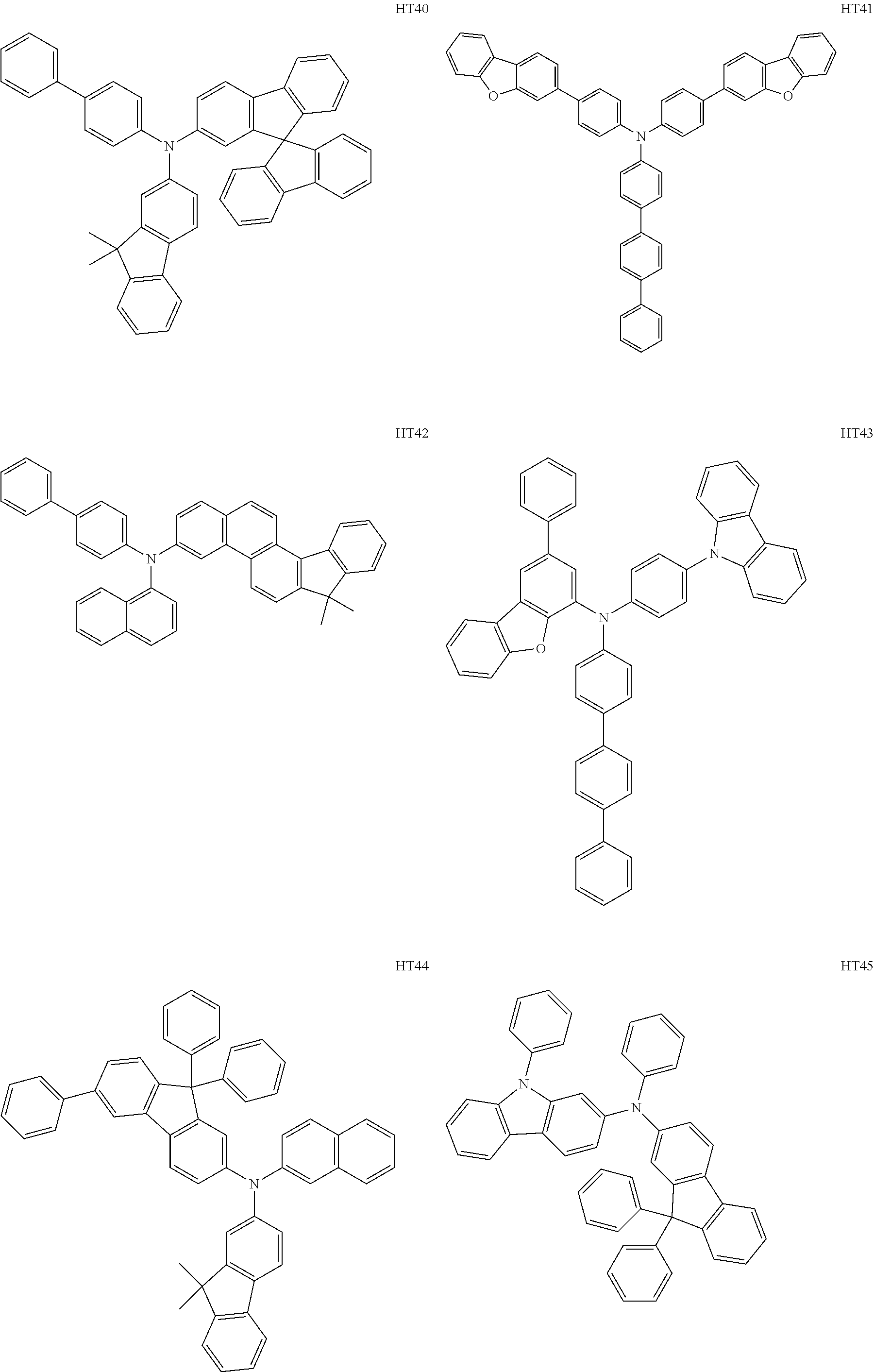

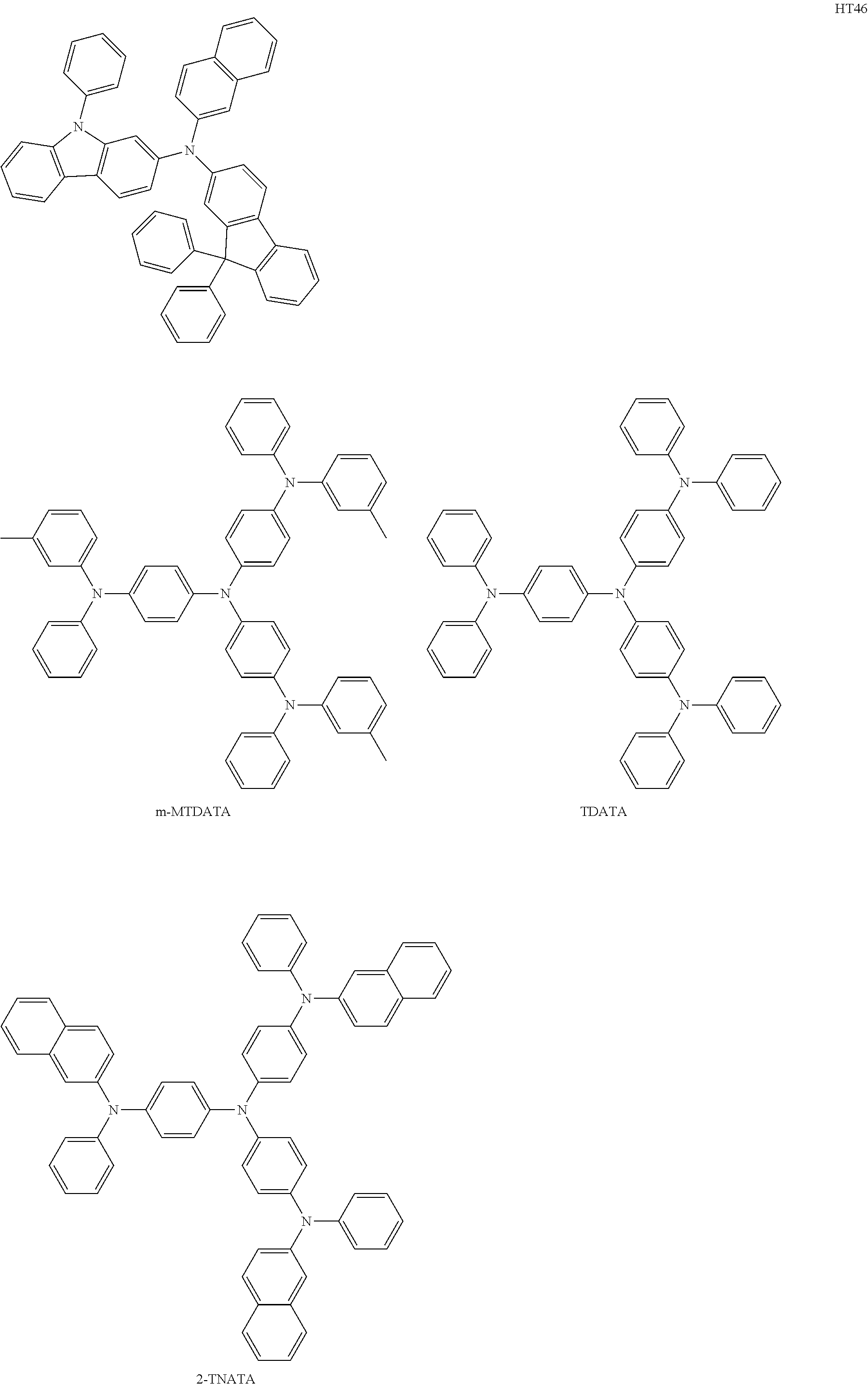

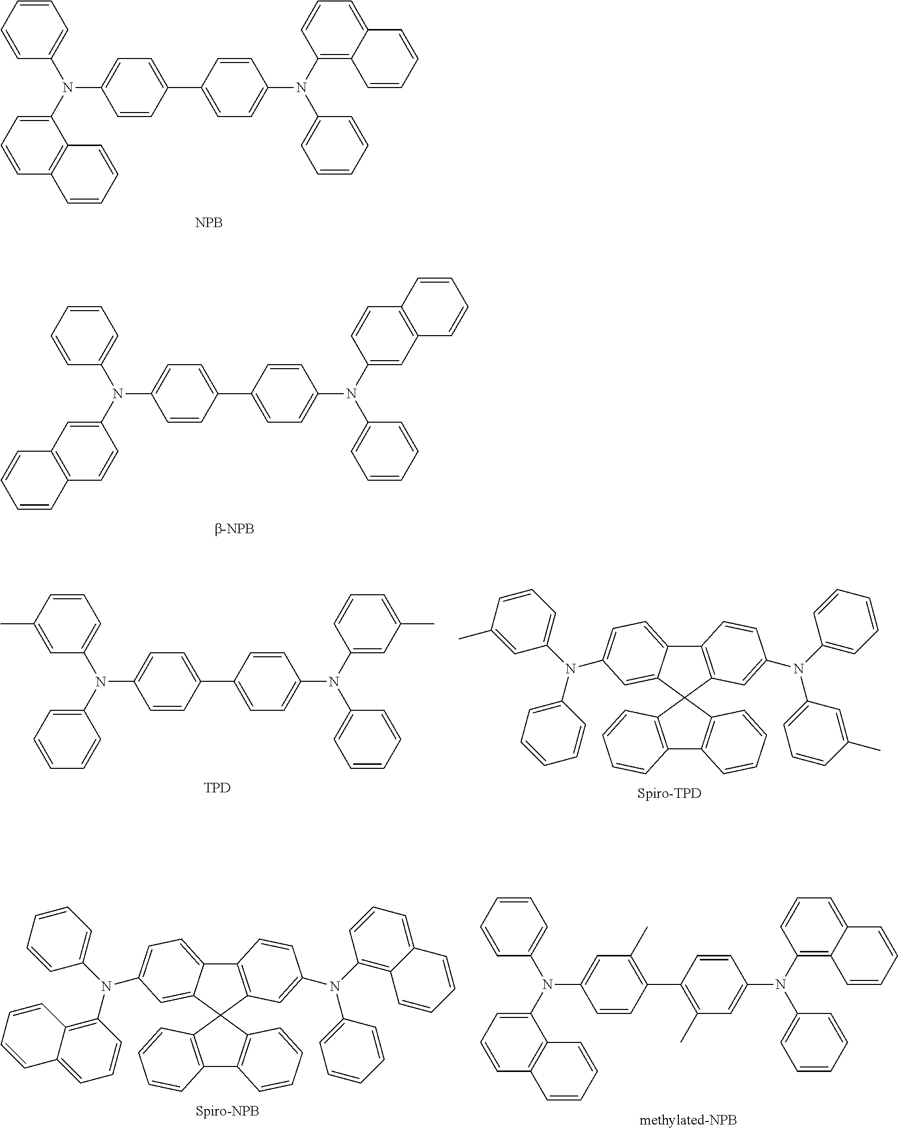

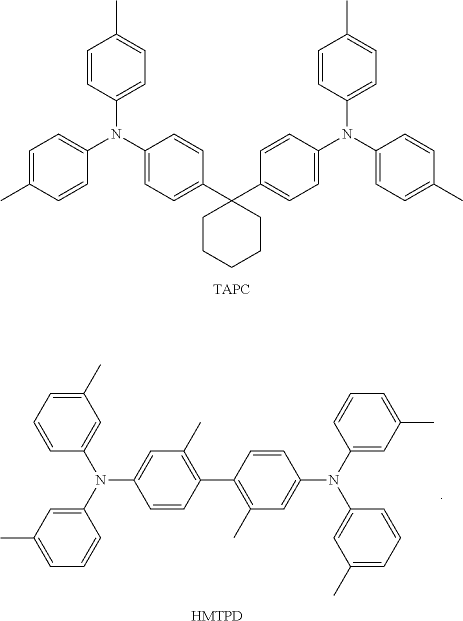

A thickness of the hole transport region may be in a range of about 50 Å to about 10,000 Å, for example, about 100 Å to about 4,000 Å. When the hole transport region includes a hole injection layer, a hole transport layer, or any suitable combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer and the hole transport layer are within these ranges, satisfactory (suitable) hole-transporting characteristics may be obtained without a substantial increase in driving voltage.

The emission auxiliary layer may increase light-emission efficiency by compensating for an optical resonance distance according to the wavelength of light emitted by an emission layer, and the electron blocking layer may block (reduce) the leakage of electrons from an emission layer to a hole transport region. Materials that may be included in the hole transport region may be included in the emission auxiliary layer and the electron blocking layer.

p-Dopant

The hole transport region may further include, in addition to these materials, a charge-generation material for the improvement of conductive properties. The charge-generation material may be substantially uniformly or non-uniformly dispersed in the hole transport region (for example, in the form of a single layer including a charge-generation material).

The charge-generation material may be, for example, a p-dopant.

In an embodiment, a LUMO energy level of the p-dopant may be about −3.5 eV or less.

In an embodiment, the p-dopant may include a quinone derivative, a cyano group-containing compound, a compound containing element EL1 and element EL2, or any suitable combination thereof.

Examples of the quinone derivative may include TCNQ, F4-TCNQ, and/or the like.

Examples of the cyano group-containing compound may include HAT-CN, a compound represented by Formula 221 below, and/or the like.

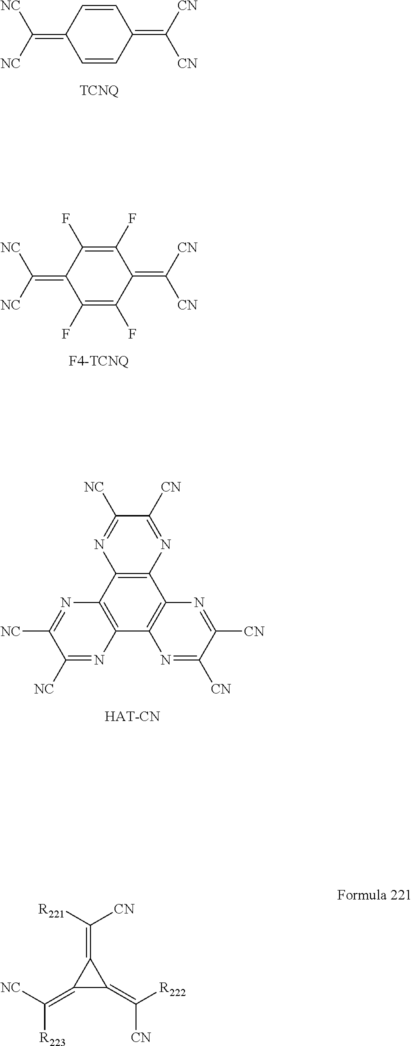

In Formula 221,

R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and

at least one of R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any suitable combinations thereof.

In the compound containing element EL1 and element EL2, element EL1 may be metal, metalloid, or a combination thereof, and element EL2 may be non-metal, metalloid, or a combination thereof.

Examples of the metal may include an alkali metal (for example, lithium (Li), sodium (Na), potassium (K), rubidium (Rb), cesium (Cs), and the like.); an alkaline earth metal (for example, beryllium (Be), magnesium (Mg), calcium (Ca), strontium (Sr), barium (Ba), and the like); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), and the like); a post-transition metal (for example, zinc (Zn), indium (In), tin (Sn), and the like); and a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and/or the like).

Examples of the metalloid may include silicon (Si), antimony (Sb), and/or tellurium (Te).

Examples of the non-metal may include oxygen (O) and/or halogen (for example, F, Cl, Br, I, etc.).

In an embodiment, examples of the compound containing element EL1 and element EL2 may include metal oxide, metal halide (for example, metal fluoride, metal chloride, metal bromide, or metal iodide), metalloid halide (for example, metalloid fluoride, metalloid chloride, metalloid bromide, or metalloid iodide), metal telluride, or any suitable combinations thereof.

Examples of the metal oxide may include tungsten oxide (for example, WO, W₂O₃, WO₂, WO₃, W₂O₅, etc.), vanadium oxide (for example, VO, V₂O₃, VO₂, V₂O₅, etc.), molybdenum oxide (MoO, Mo₂O₃, MoO₂, MoO₃, Mo₂O₅, etc.), and rhenium oxide (for example, ReO₃, etc.).

Examples of the metal halide may include an alkali metal halide, an alkaline earth metal halide, a transition metal halide, a post-transition metal halide, and/or a lanthanide metal halide.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, and/or CsI.

Examples of the alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, and/or BaI₂.

Examples of the transition metal halide may include titanium halide (for example, TiF₄, TiCl₄, TiBr₄, Til₄, etc.), zirconium halide (for example, ZrF₄, ZrCl₄, ZrBr₄, ZrI₄, etc.), hafnium halide (for example, HfF₄, HfCl₄, HfBr₄, HfI₄, etc.), vanadium halide (for example, VF₃, VCl₃, VBr₃, VI₃, etc.), niobium halide (for example, NbF₃, NbCl₃, NbBr₃, Nbl₃, etc.), tantalum halide (for example, TaF₃, TaCl₃, TaBr₃, TaI₃, etc.), chromium halide (for example, CrF₃, CrCl₃, CrBr₃, CrI₃, etc.), molybdenum halide (for example, MoF₃, MoCl₃, MoBr₃, MoI₃, etc.), tungsten halide (for example, WF₃, WCl₃, WBr₃, WI₃, etc.), manganese halide (for example, MnF₂, MnCl₂, MnBr₂, MnI₂, etc.), technetium halide (for example, TcF₂, TcCl₂, TcBr₂, TcI₂, etc.), rhenium halide (for example, ReF₂, ReCl₂, ReBr₂, ReI₂, etc.), iron halide (for example, FeF₂, FeCl₂, FeBr₂, FeI₂, etc.), ruthenium halide (for example, RuF₂, RuCl₂, RuBr₂, RuI₂, etc.), osmium halide (for example, OsF₂, OsCl₂, OsBr₂, OsI₂, etc.), cobalt halide (for example, CoF₂, CoCl₂, CoBr₂, CoI₂, etc.), rhodium halide (for example, RhF₂, RhCl₂, RhBr₂, Rhl₂, etc.), iridium halide (for example, IrF₂, IrCl₂, IrBr₂, IrI₂, etc.), nickel halide (for example, NiF₂, NiCl₂, NiBr₂, NiI₂, etc.), palladium halide (for example, PdF₂, PdCl₂, PdBr₂, PdI₂, etc.), platinum halide (for example, PtF₂, PtCl₂, PtBr₂, PtI₂, etc.), copper halide (for example, CuF, CuCl, CuBr, CuI, etc.), silver halide (for example, AgF, AgCl, AgBr, AgI, etc.), and/or gold halide (for example, AuF, AuCl, AuBr, AuI, etc.).

Examples of the post-transition metal halide may include zinc halide (for example, ZnF₂, ZnCl₂, ZnBr₂, ZnI₂, etc.), indium halide (for example, InI₃, etc.), and/or tin halide (for example, SnI₂, etc.).

Examples of the lanthanide metal halide may include YbF, YbF₂, YbF₃, SmF₃, YbCl, YbCl₂, YbCl₃ SmCl₃, YbBr, YbBr₂, YbBr₃, SmBr₃, Ybl, Ybl₂, Ybl₃, and/or SmI₃.

Examples of the metalloid halide may include antimony halide (for example, SbCl₅, etc.).

Examples of the metal telluride may include alkali metal telluride (for example, Li₂Te, Na₂Te, K₂Te, Rb₂Te, Cs₂Te, etc.), alkaline earth metal telluride (for example, BeTe, MgTe, CaTe, SrTe, BaTe, etc.), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post-transition metal telluride (for example, ZnTe, etc.), and/or lanthanide metal telluride (for example, LaTe, CeTe, PrTe, NdTe, PmTe, EuTe, GdTe, TbTe, DyTe, HoTe, ErTe, TmTe, YbTe, LuTe, etc.).

### Emission Layer in Interlayer 130

When the light-emitting device 10 is a full-color light-emitting device, the emission layer may be patterned into a red emission layer, a green emission layer, and/or a blue emission layer, according to a sub-pixel. In an embodiment, the emission layer may have a stacked structure of two or more layers of a red emission layer, a green emission layer, and a blue emission layer, in which the two or more layers contact each other or are separated from each other. In one or more embodiments, the emission layer may include two or more materials of a red light-emitting material, a green light-emitting material, and a blue light-emitting material, in which the two or more materials are mixed with each other in a single layer to emit white light.

The emission layer may include a host and a dopant. The dopant may include a phosphorescent dopant, a fluorescent dopant, or any suitable combination thereof.

An amount of the dopant in the emission layer may be from about 0.01 parts by weight to about 15 parts by weight based on 100 parts by weight of the host.

In an embodiment, the emission layer may include a quantum dot.

In an embodiment, the emission layer may include a delayed fluorescence material. The delayed fluorescence material may act as a host or a dopant in the emission layer.

A thickness of the emission layer may be in a range of about 100 Å to about 1,000 Å, for example, about 200 Å to about 600 Å. When the thickness of the emission layer is within the range, excellent light-emission characteristics may be obtained without a substantial increase in driving voltage.

### Host

The host may include a compound represented by Formula 301 below:

[Ar₃₀₁]_{xb11}-[(L₃₀₁)_{xb1}-R₃₀₁]_{xb21} Formula 301

wherein, in Formula 301,

Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xb11 may be 1, 2, or 3,

xb1 may be an integer from 0 to 5,

R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

xb21 may be an integer from 1 to 5, and

Q₃₀₁ to Q₃₀₃ are the same as described in connection with Q₁.

For example, when xb11 in Formula 301 is 2 or more, two or more of Ar₃₀₁ may be

linked together via a single bond.

In an embodiment, the host may include a compound represented by Formula 301-1, a compound represented by Formula 301-2, or any suitable combinations thereof:

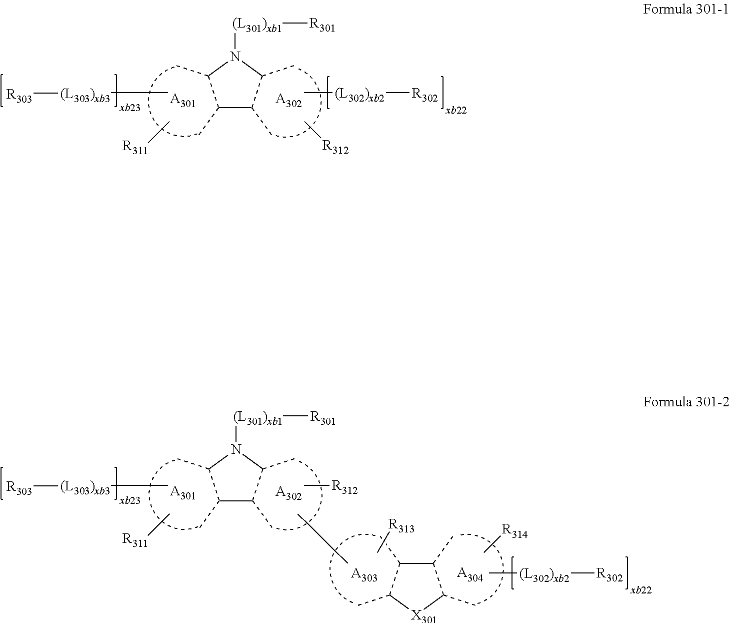

wherein, in Formulae 301-1 and 301-2,

ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

X₃₀₁ may be O, S, N-[(L₃₀₄)_{xb4}-R₃₀₄], C(R₃₀₄)(R₃₀₅), or Si(R₃₀₄)(R₃₀₅),

xb22 and xb23 may each independently be 0, 1, or 2,

L₃₀₁, xb1, and R₃₀₁ are respectively the same as those described in the present disclosure,

L₃₀₂ to L₃₀₄ are each independently the same as described in connection with L₃₀₁,

xb2 to xb4 are each independently the same as described in connection with xb1, and

R₃₀₂ to R₃₀₅ and R₃₁₁ to R₃₁₄ are respectively the same as those described in connection with R₃₀₁.

In an embodiment, the host may include an alkali earth metal complex, a post-transition metal complex, or a combination thereof. In an embodiment, the host may include a Be complex (for example, Compound H55), an Mg complex, a Zn complex, or a combination thereof.

In an embodiment, the host may include one of Compounds H1 to H124, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any suitable combinations thereof:

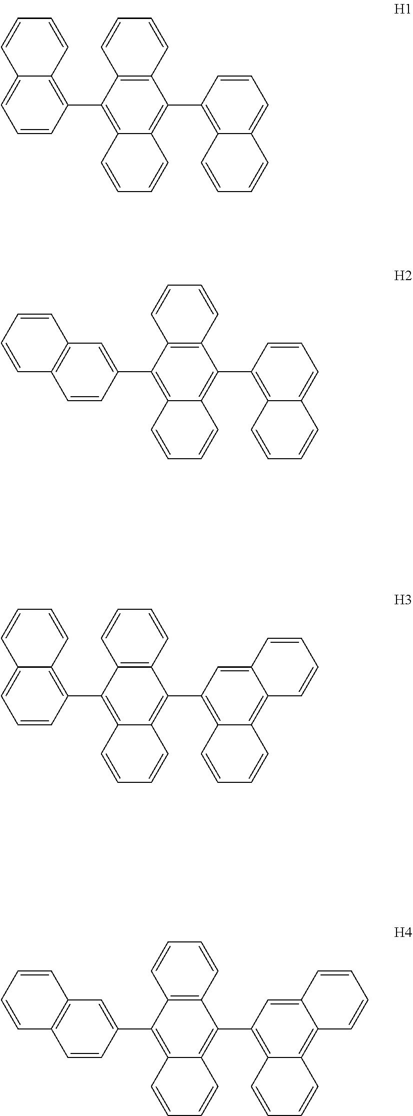

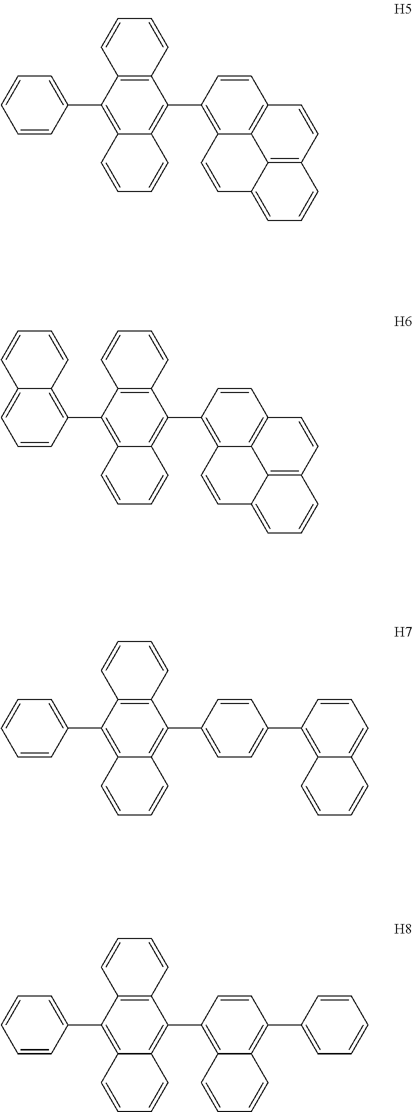

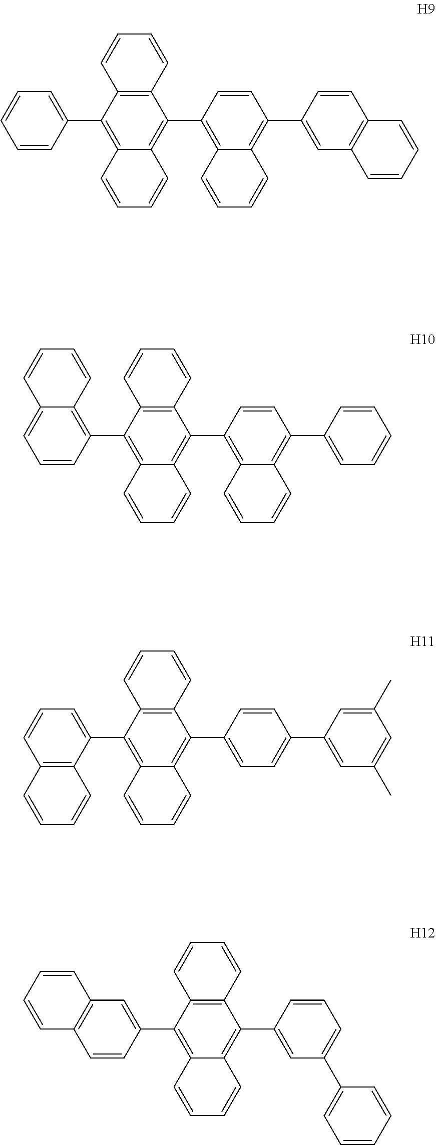

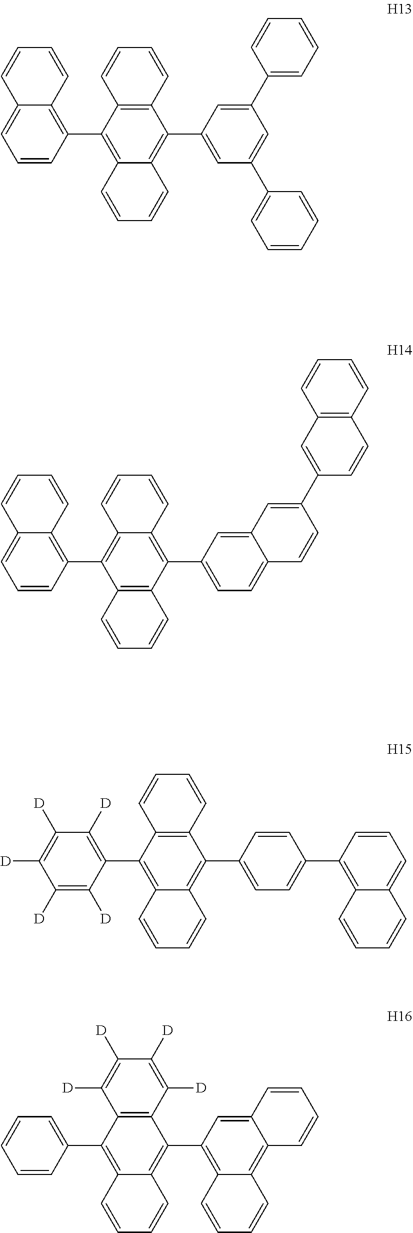

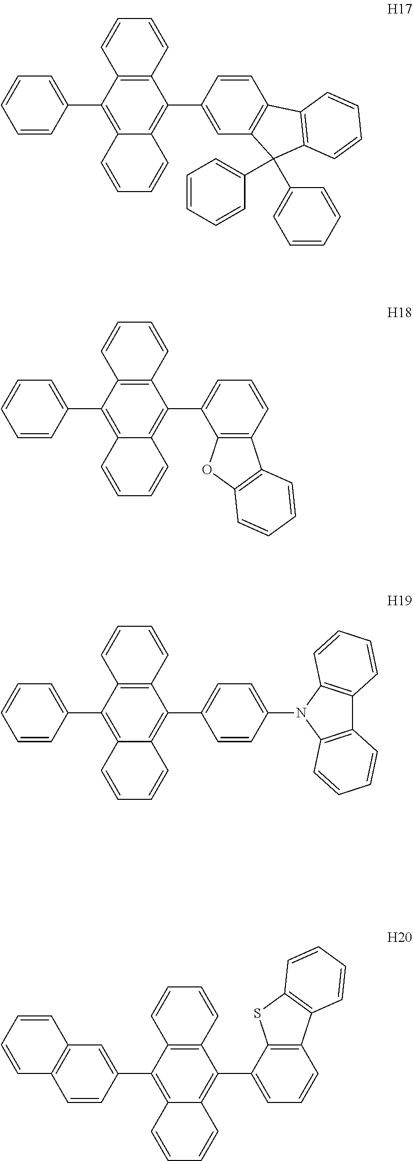

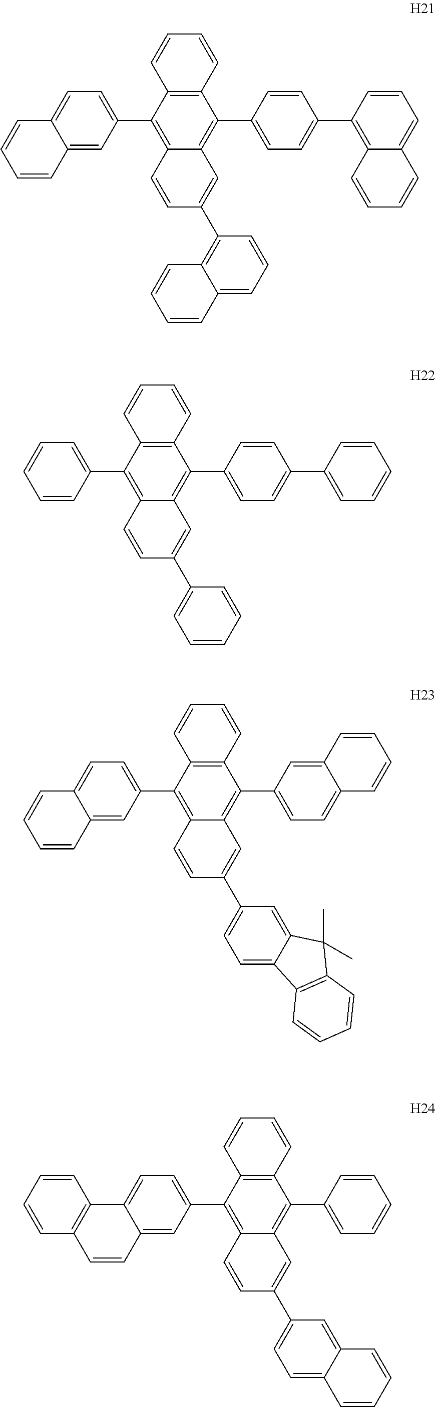

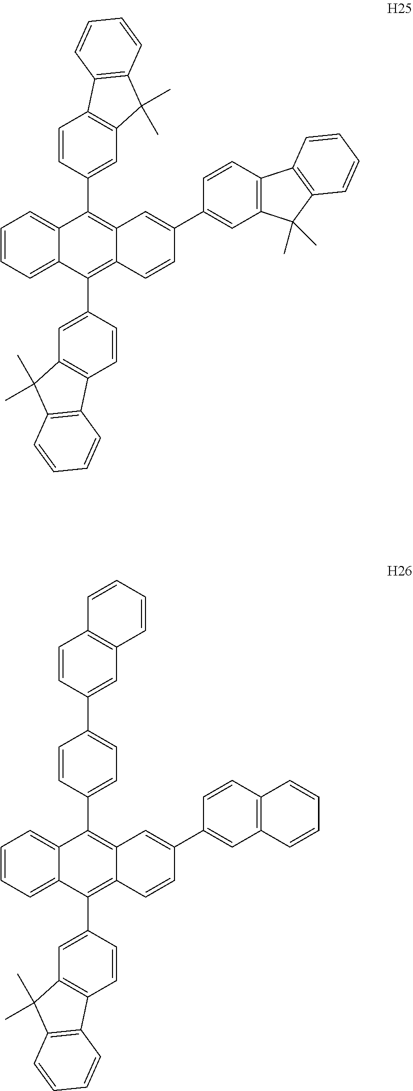

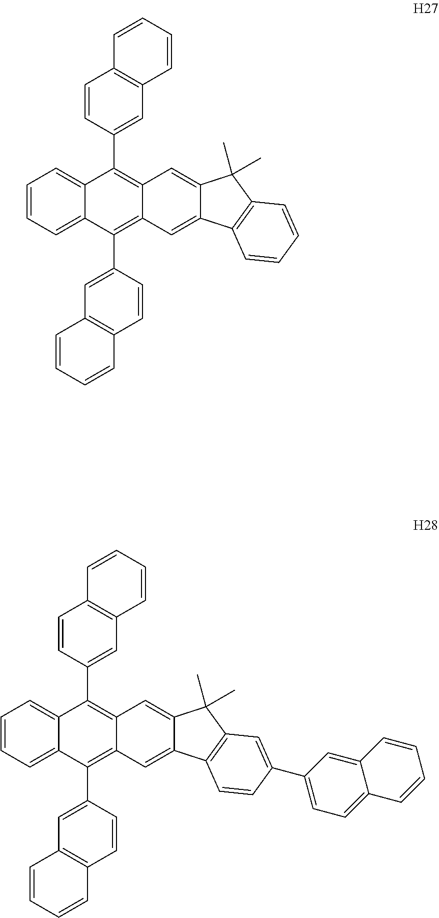

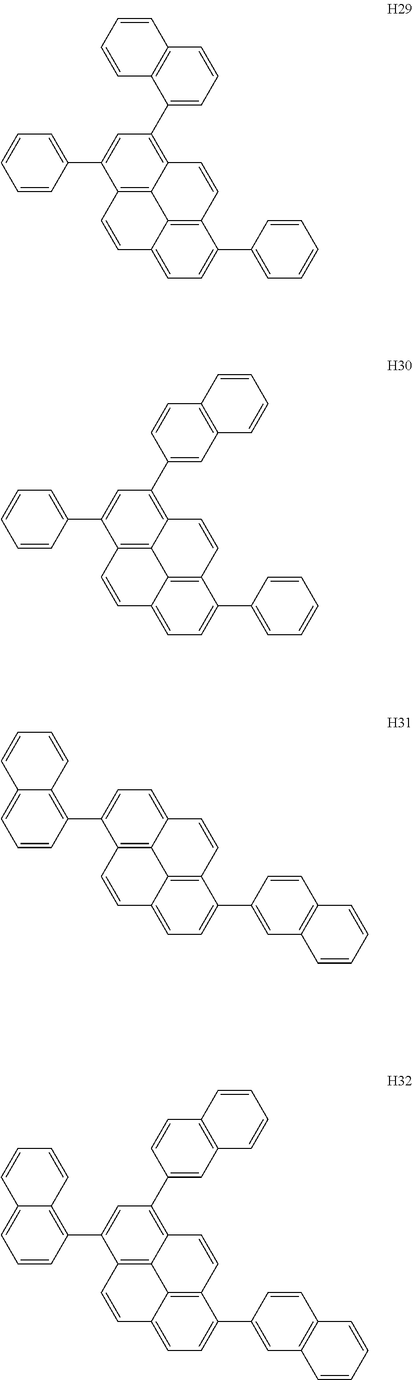

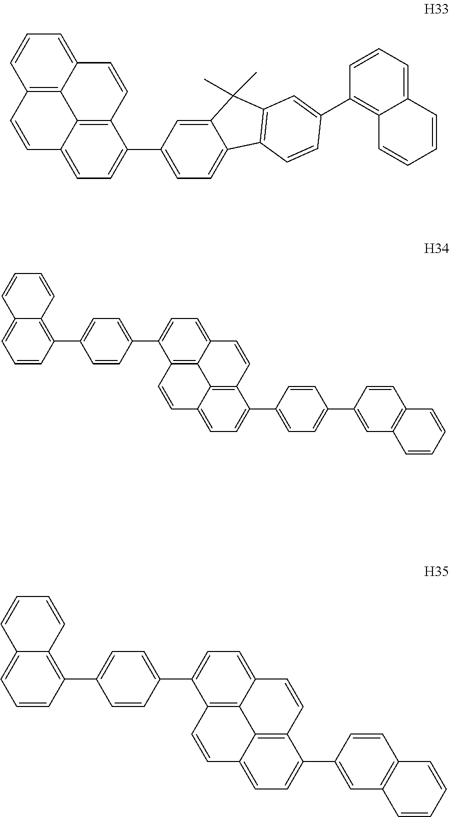

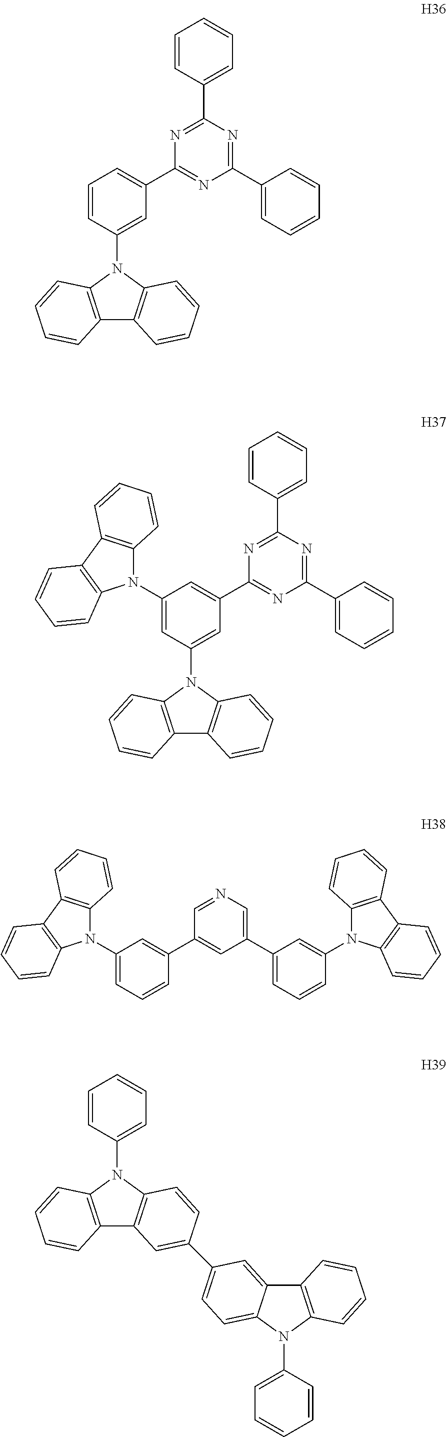 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1]

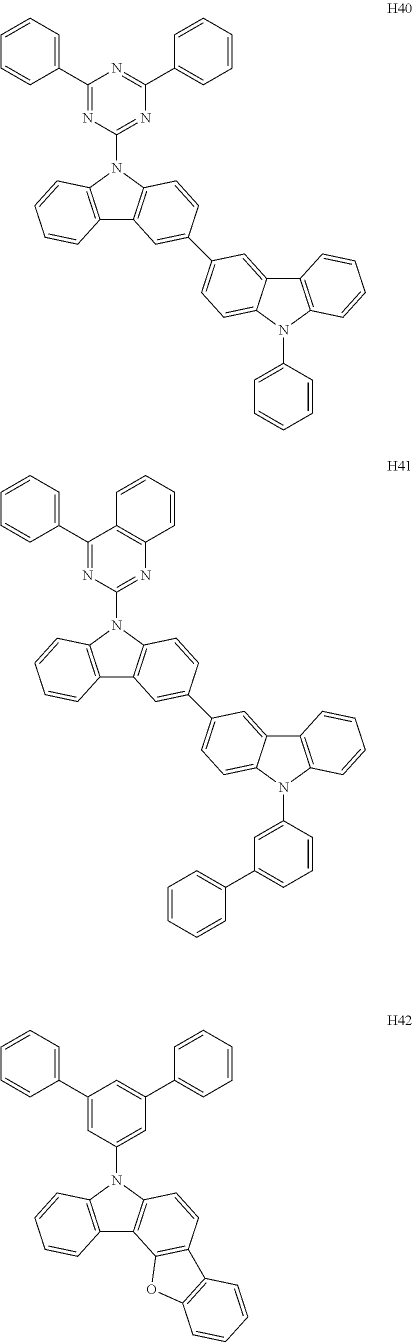 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

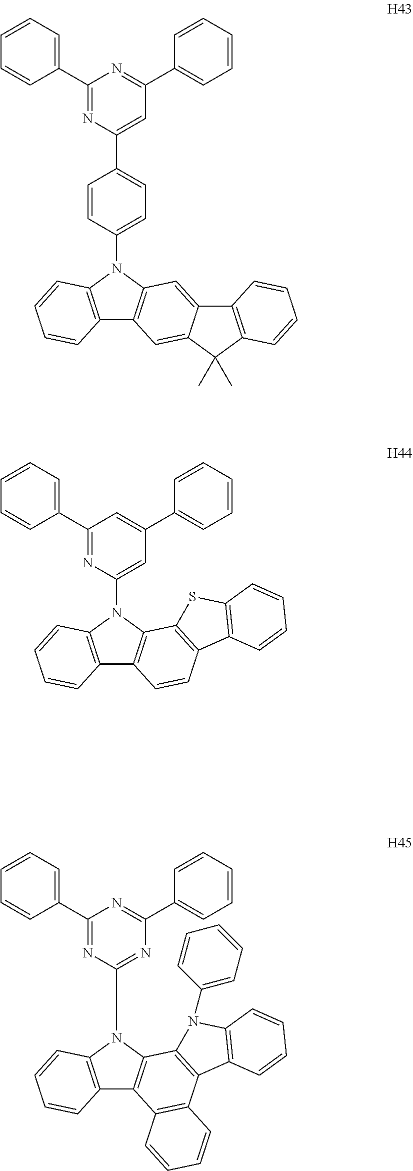 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1]

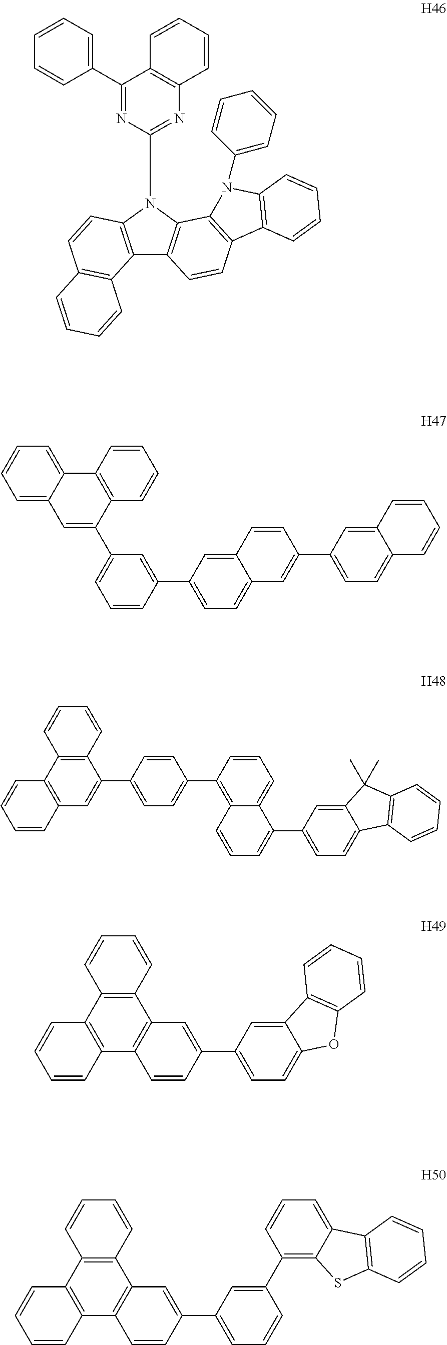

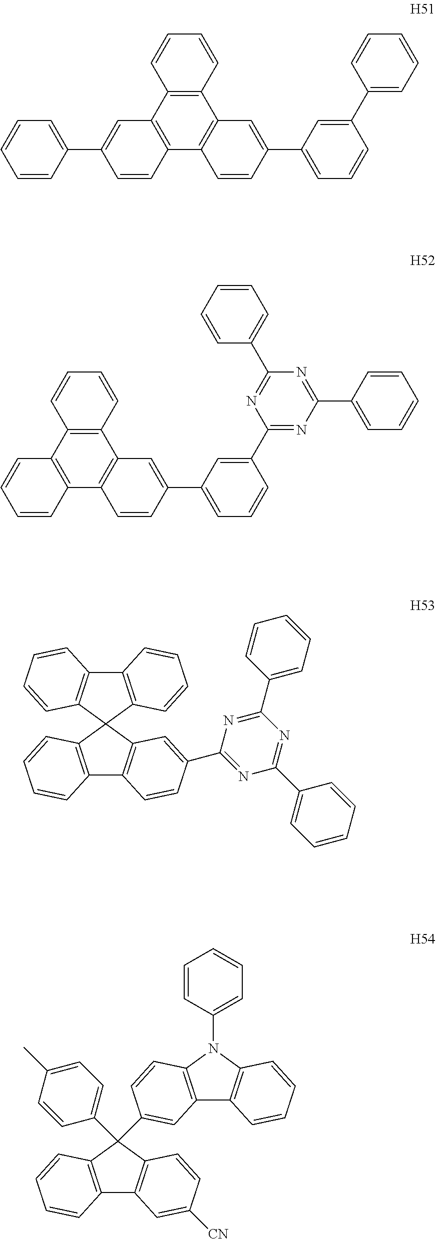

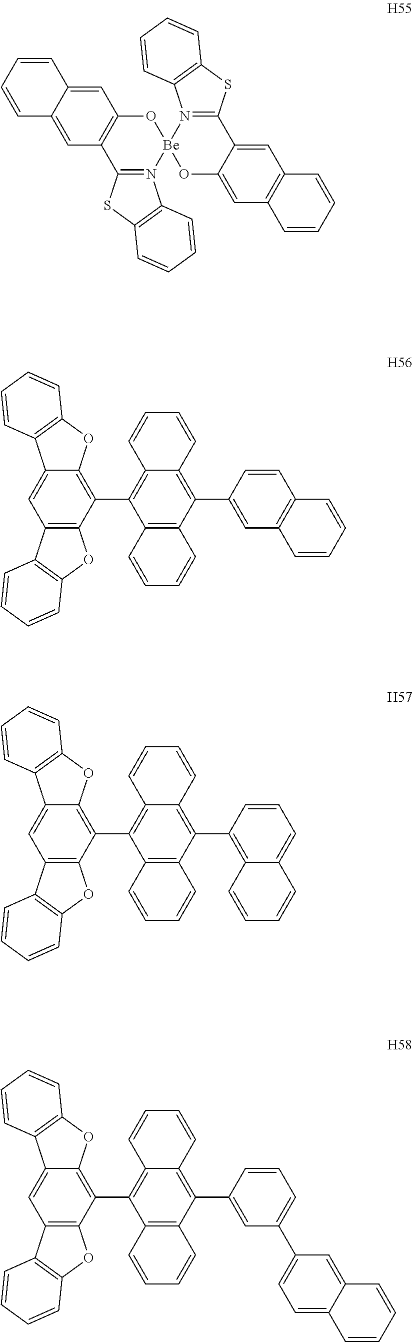

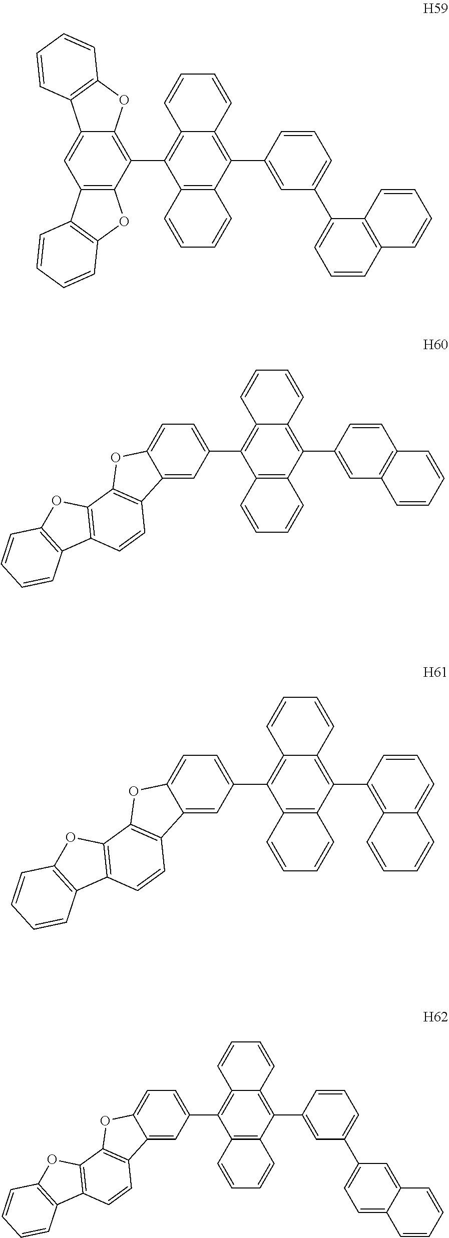 [SMILES: c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1]

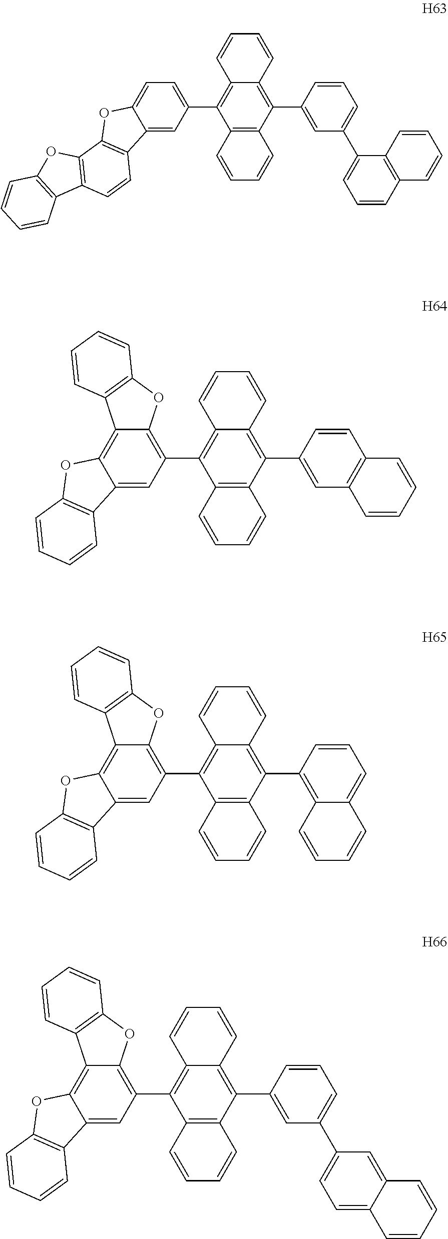 [SMILES: c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1 ; c1ccc2c(-c3c4ccccc4c(-c4cc5c6ccccc6oc5c5c4oc4ccccc45)c4ccccc34)cccc2c1 ; c1cc(-c2ccc3ccccc3c2)cc(-c2c3ccccc3c(-c3cc4c5ccccc5oc4c4c3oc3ccccc34)c3ccccc23)c1]

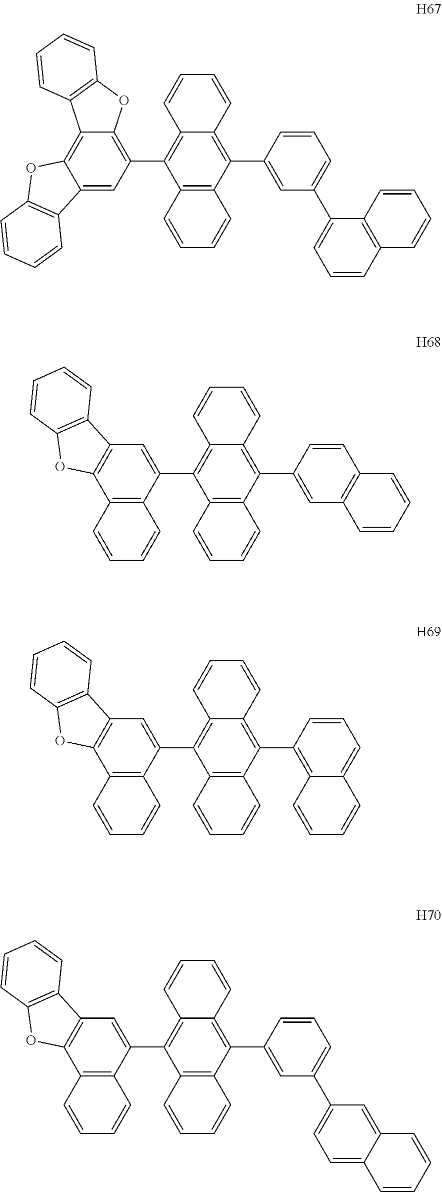 [SMILES: c1cc(-c2cccc3ccccc23)cc(-c2c3ccccc3c(-c3cc4c5ccccc5oc4c4c3oc3ccccc34)c3ccccc23)c1 ; c1ccc2cc(-c3c4ccccc4c(-c4cc5c6ccccc6oc5c5ccccc45)c4ccccc34)ccc2c1 ; c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1]

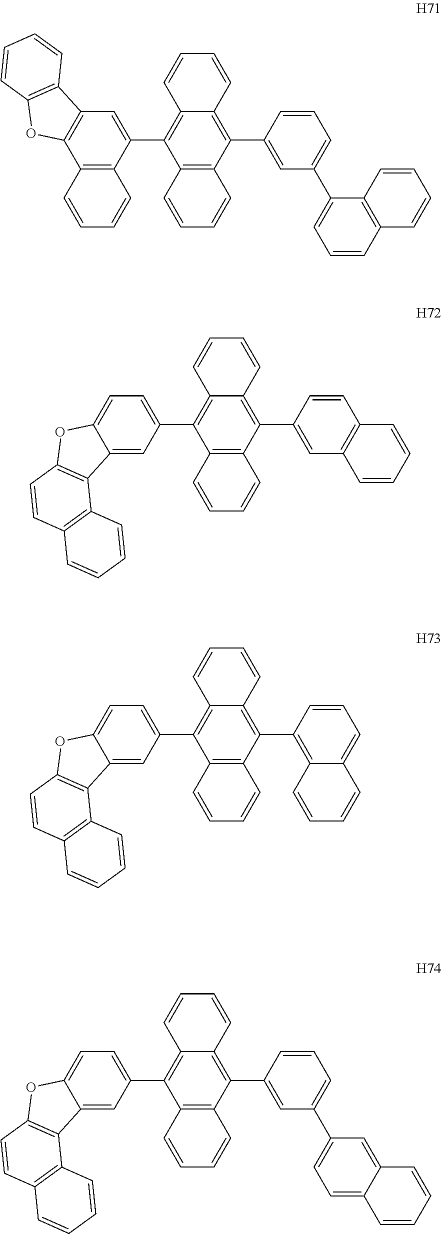 [SMILES: c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1 ; c1ccc2c(-c3c4ccccc4c(-c4ccc5oc6ccc7ccccc7c6c5c4)c4ccccc34)cccc2c1 ; c1cc(-c2ccc3ccccc3c2)cc(-c2c3ccccc3c(-c3ccc4oc5ccc6ccccc6c5c4c3)c3ccccc23)c1]

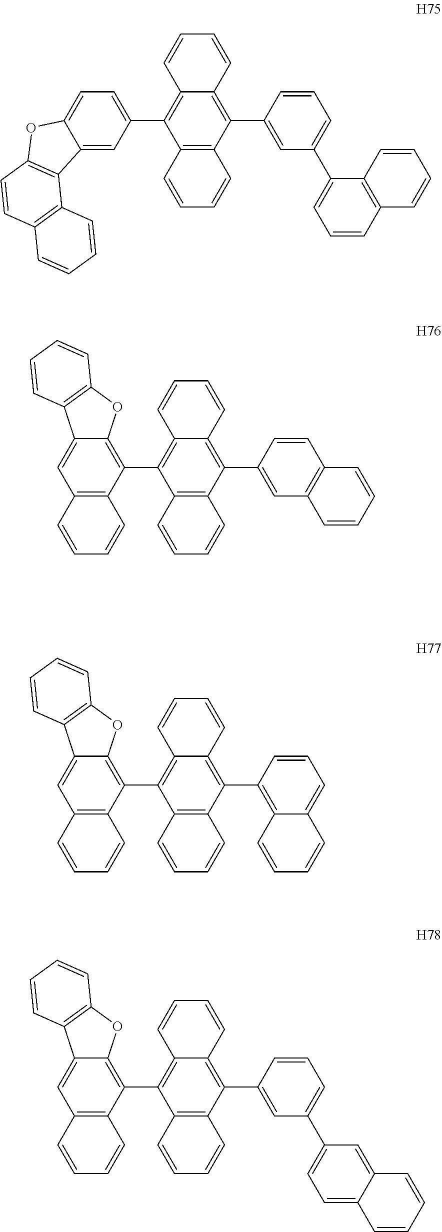 [SMILES: c1cc(-c2cccc3ccccc23)cc(-c2c3ccccc3c(-c3ccc4oc5ccc6ccccc6c5c4c3)c3ccccc23)c1 ; c1ccc2cc(-c3c4ccccc4c(-c4c5ccccc5cc5c4oc4ccccc45)c4ccccc34)ccc2c1 ; c1ccc2c(-c3c4ccccc4c(-c4c5ccccc5cc5c4oc4ccccc45)c4ccccc34)cccc2c1 ; c1cc(-c2ccc3ccccc3c2)cc(-c2c3ccccc3c(-c3c4ccccc4cc4c3oc3ccccc34)c3ccccc23)c1]

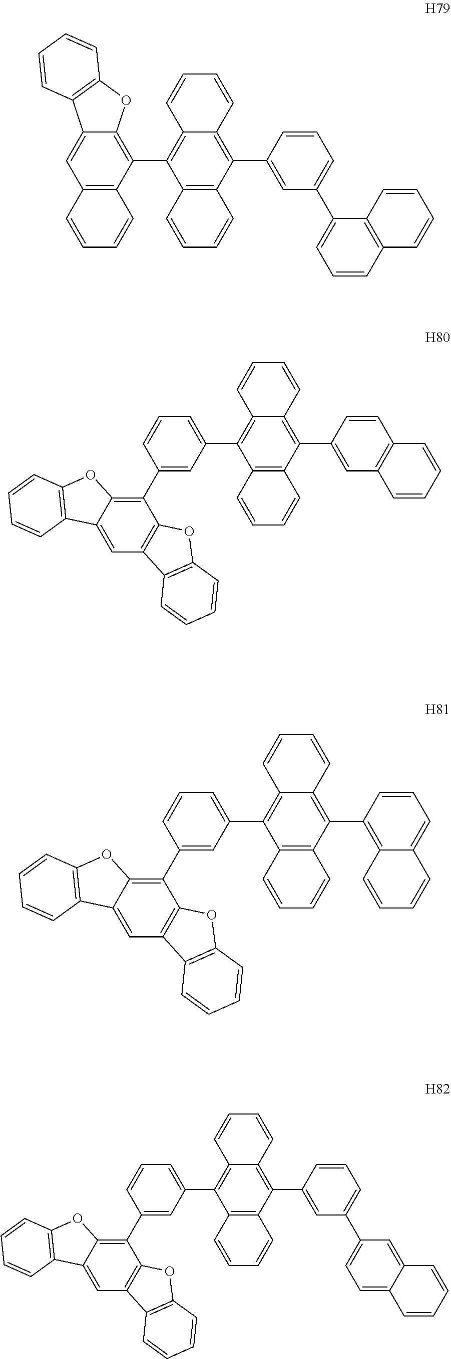 [SMILES: c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1 ; c1cc(-c2ccc3ccccc3c2)cc(-c2c3ccccc3c(-c3cccc(-c4c5oc6ccccc6c5cc5c4oc4ccccc45)c3)c3ccccc23)c1]

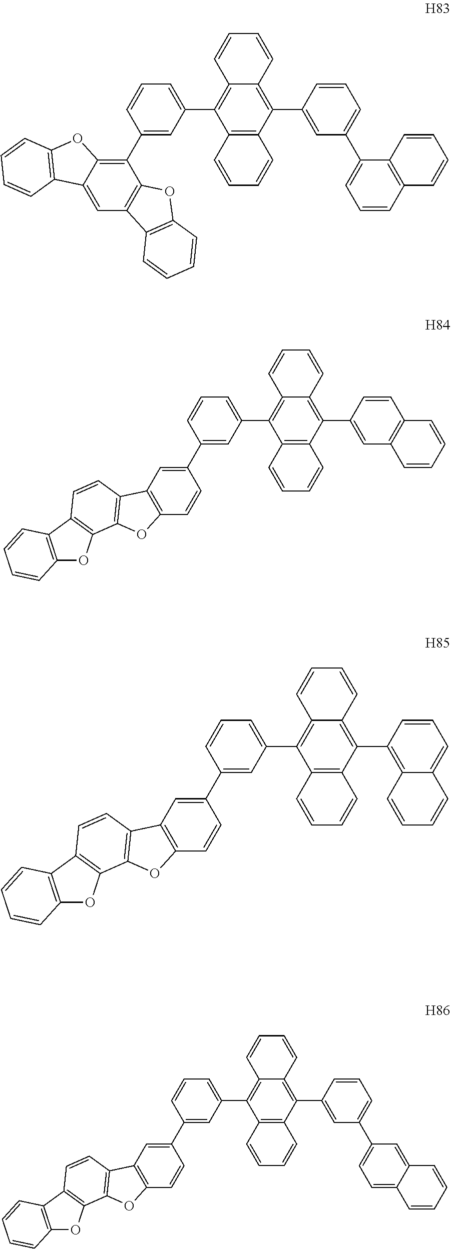 [SMILES: c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1]

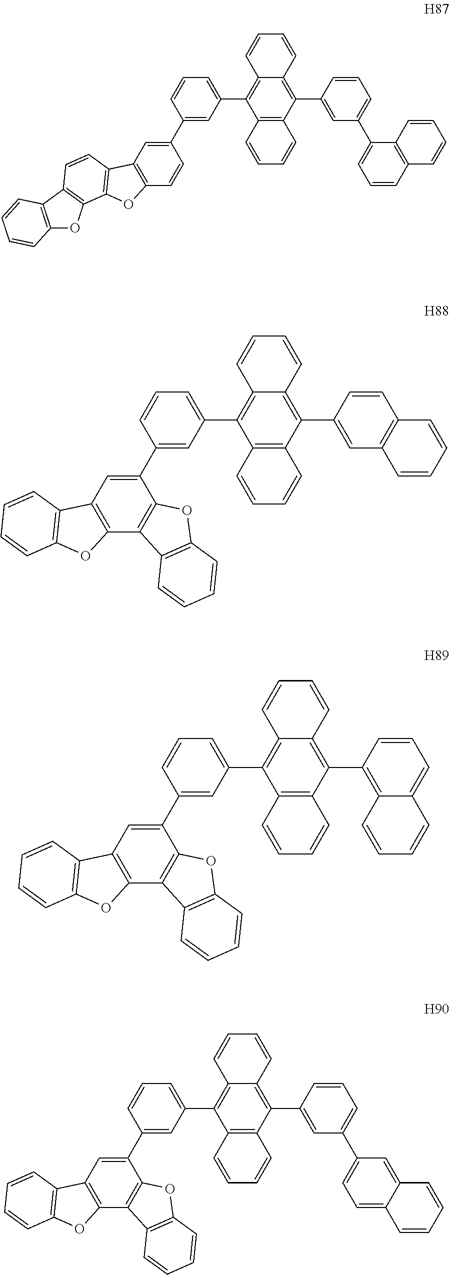 [SMILES: c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1]

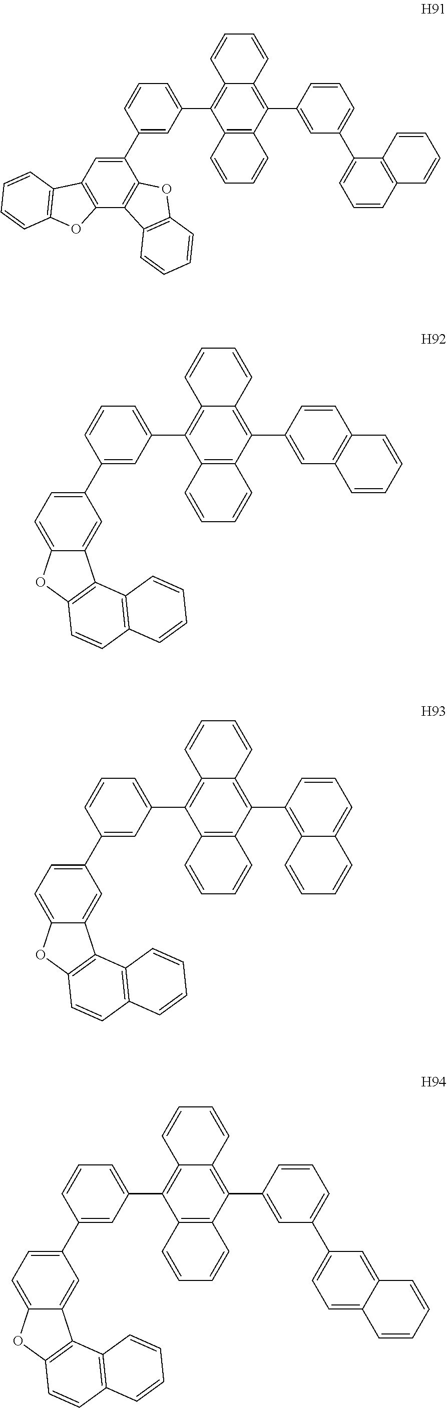 [SMILES: c1cc(-c2cccc3ccccc23)cc(-c2c3ccccc3c(-c3cccc(-c4cc5c6ccccc6oc5c5c4oc4ccccc45)c3)c3ccccc23)c1 ; c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1]

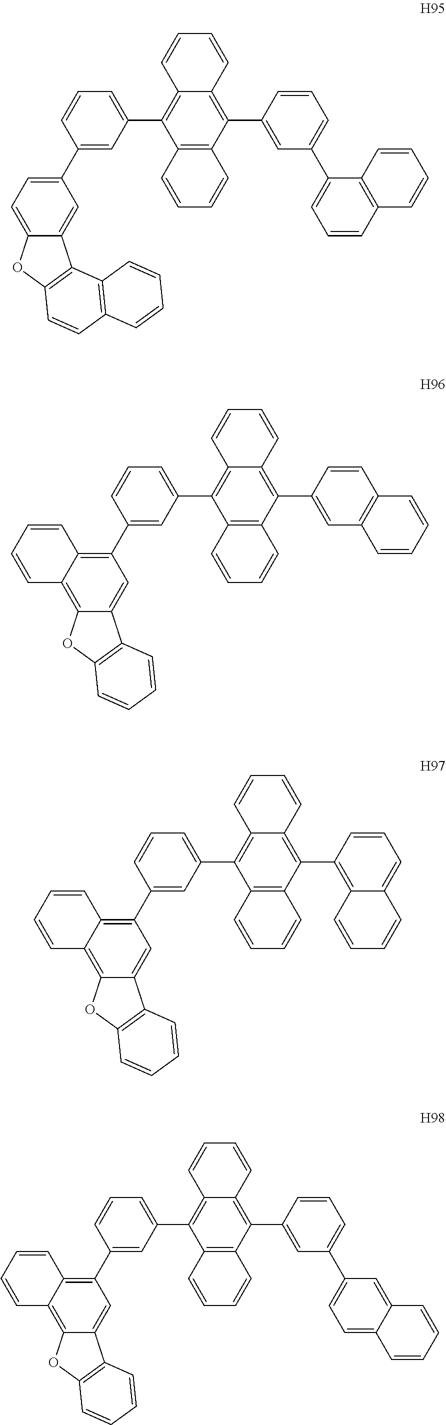 [SMILES: c1cc(-c2ccc3oc4ccc5ccccc5c4c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1]

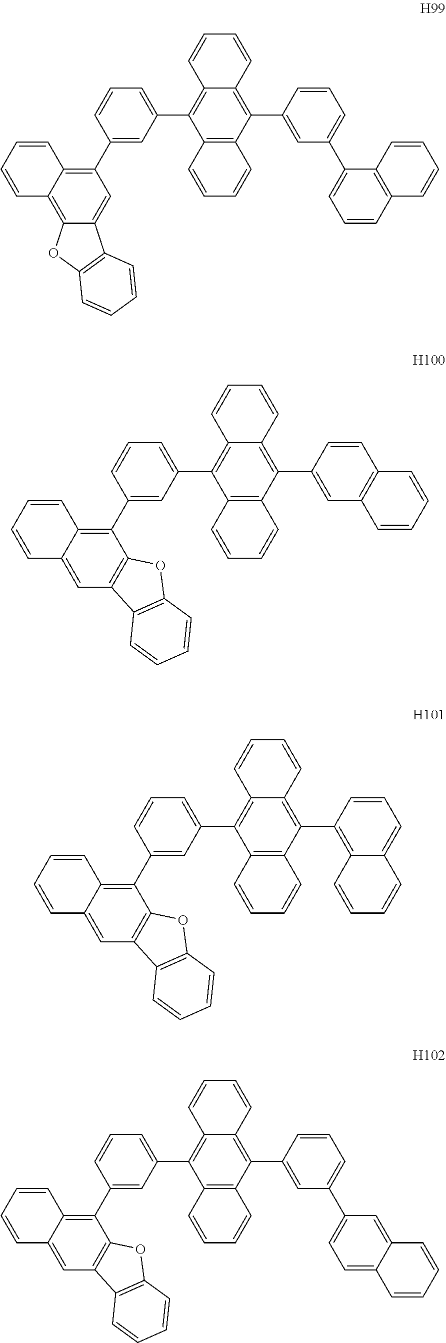 [SMILES: c1cc(-c2cccc3ccccc23)cc(-c2c3ccccc3c(-c3cccc(-c4cc5c6ccccc6oc5c5ccccc45)c3)c3ccccc23)c1 ; c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1]

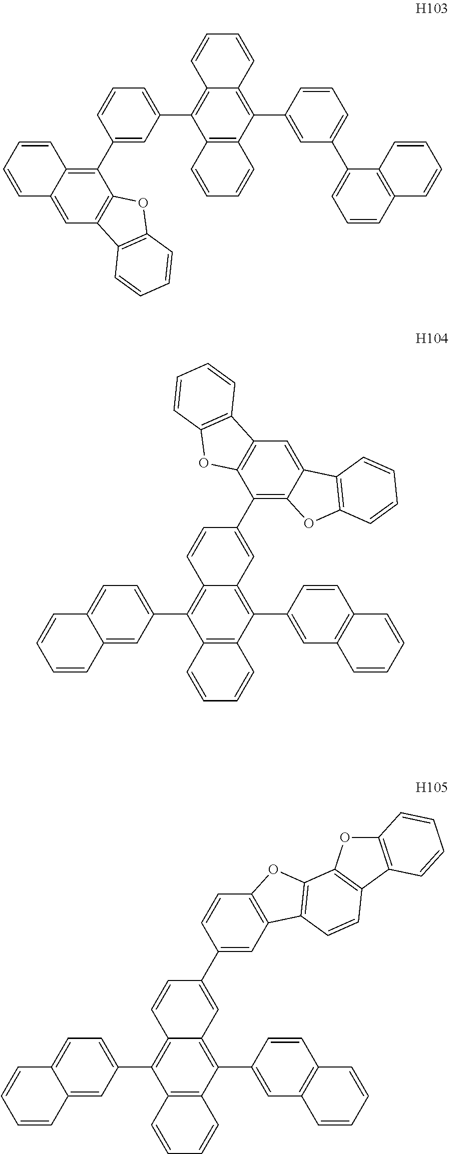 [SMILES: c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1 ; c1ccc2cc(-c3c4ccccc4c(-c4ccc5ccccc5c4)c4cc(-c5ccc6oc7c(ccc8c9ccccc9oc87)c6c5)ccc34)ccc2c1]

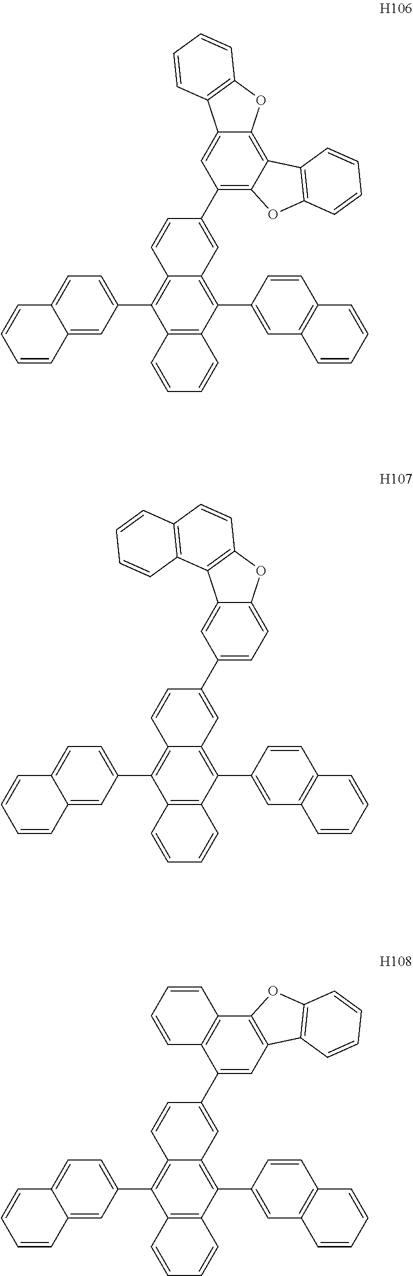 [SMILES: c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1 ; c1ccc2cc(-c3c4ccccc4c(-c4ccc5ccccc5c4)c4cc(-c5cc6c7ccccc7oc6c6ccccc56)ccc34)ccc2c1]

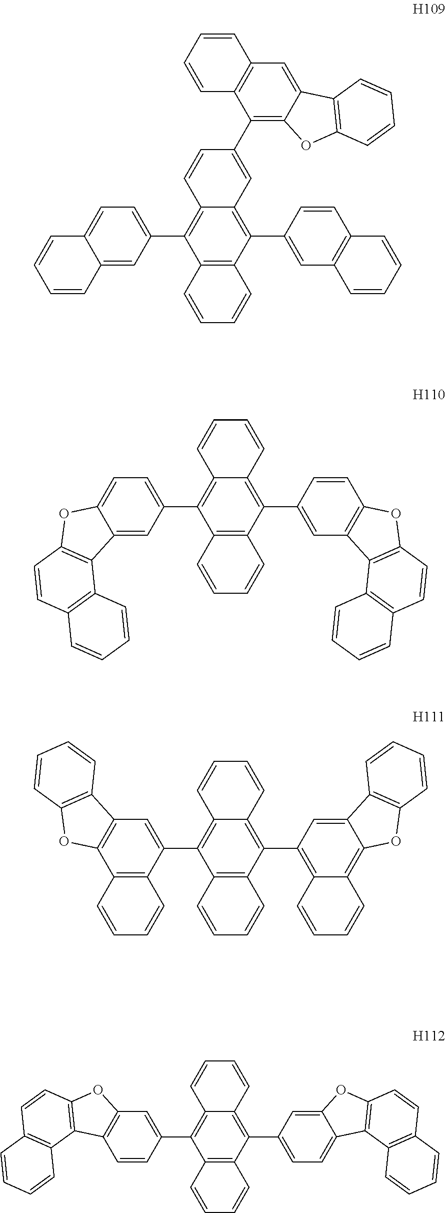 [SMILES: c1ccc2cc(-c3c4ccccc4c(-c4ccc5ccccc5c4)c4cc(-c5c6ccccc6cc6c5oc5ccccc56)ccc34)ccc2c1 ; c1ccc2c(c1)ccc1oc3ccc(-c4c5ccccc5c(-c5ccc6oc7ccc8ccccc8c7c6c5)c5ccccc45)cc3c12 ; c1ccc2c(c1)oc1c3ccccc3c(-c3c4ccccc4c(-c4cc5c6ccccc6oc5c5ccccc45)c4ccccc34)cc21 ; c1ccc2c(c1)ccc1oc3cc(-c4c5ccccc5c(-c5ccc6c(c5)oc5ccc7ccccc7c56)c5ccccc45)ccc3c12]

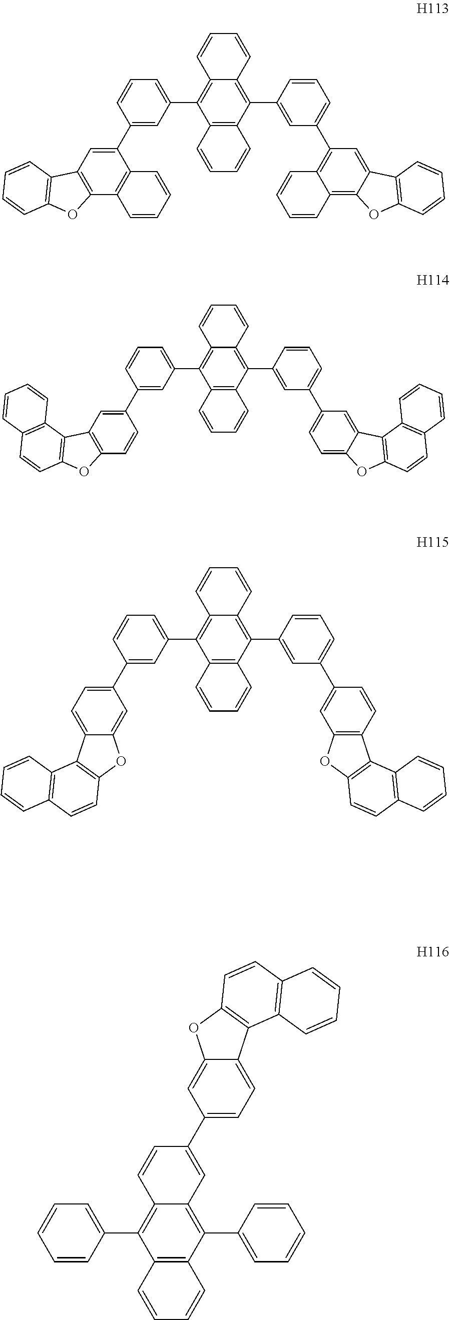 [SMILES: c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1]

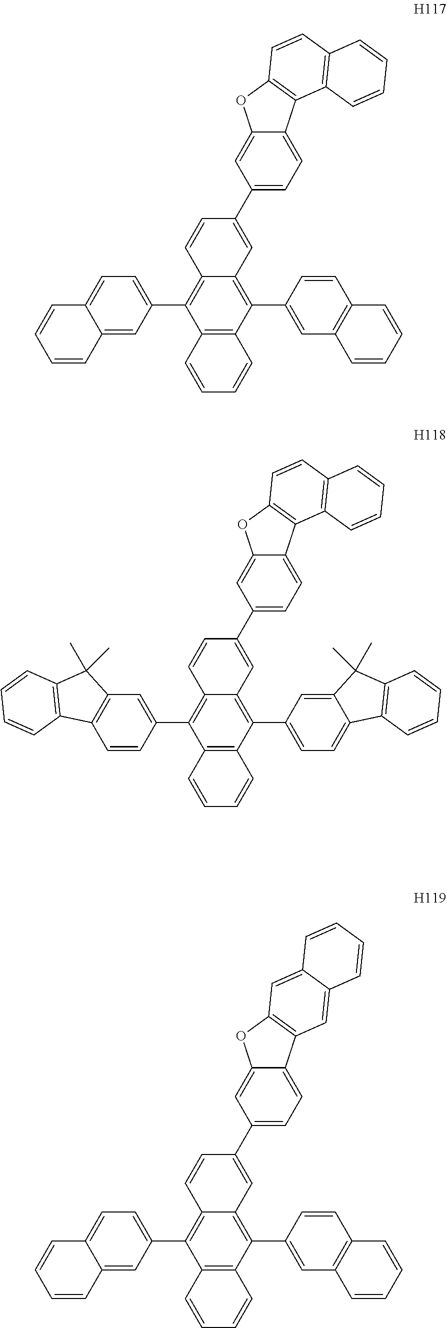 [SMILES: c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21 ; c1ccc2cc(-c3c4ccccc4c(-c4ccc5ccccc5c4)c4cc(-c5ccc6c(c5)oc5cc7ccccc7cc56)ccc34)ccc2c1]

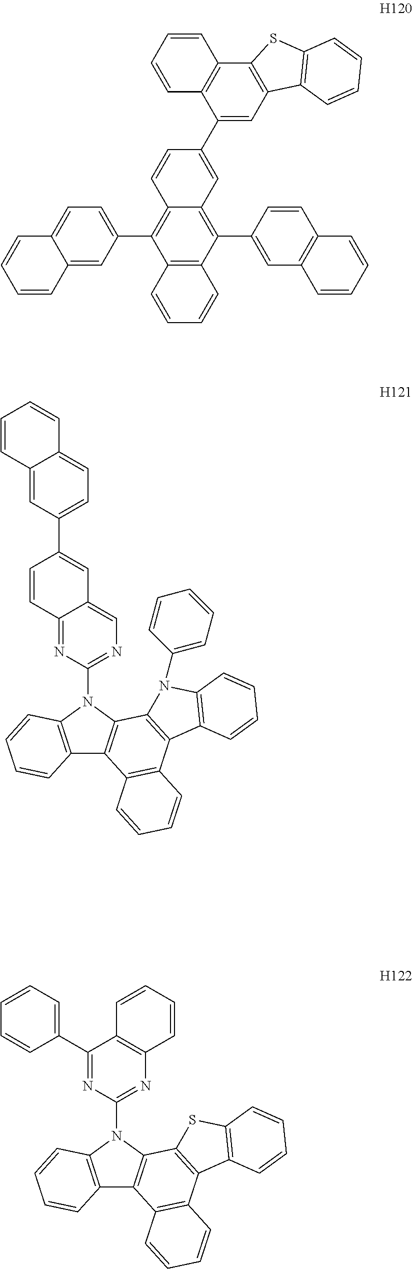 [SMILES: c1ccc2cc(-c3c4ccccc4c(-c4ccc5ccccc5c4)c4cc(-c5cc6c7ccccc7sc6c6ccccc56)ccc34)ccc2c1 ; c1ccc(-n2c3ccccc3c3c4ccccc4c4c5ccccc5n(-c5ncc6cc(-c7ccc8ccccc8c7)ccc6n5)c4c32)cc1 ; c1ccc(-c2nc(-n3c4ccccc4c4c5ccccc5c5c6ccccc6sc5c43)nc3ccccc23)cc1]

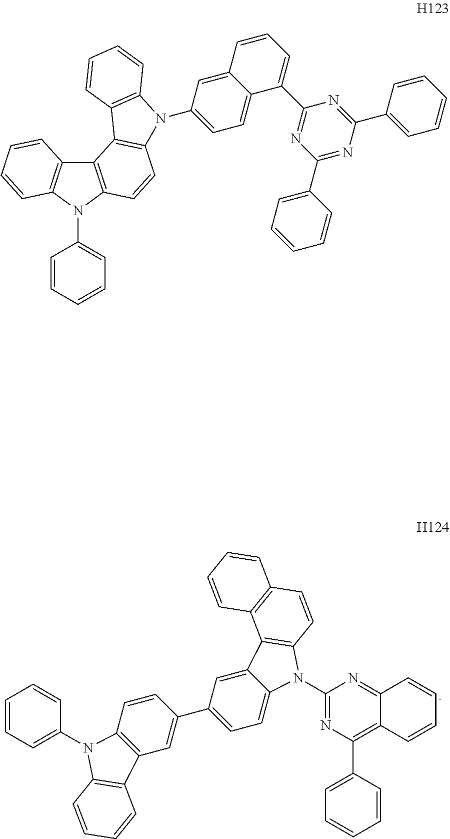 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

### Phosphorescent Dopant

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any suitable combination thereof.

The phosphorescent dopant may be electrically neutral.

In an embodiment, the phosphorescent dopant may include an organometallic compound represented by Formula 401:

M(L₄₀₁)_{xc1}(L₄₀₂)_{xc2} Formula 401

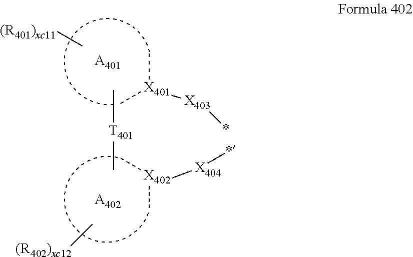

wherein, in Formulae 401 and 402,

M may be transition metal (for example, iridium (Ir), platinum (Pt), palladium (Pd), osmium (Os), titanium (Ti), gold (Au)hafnium (Hf), europium (Eu), terbium (Tb), rhodium (Rh), rhenium (Re), or thulium (Tm)),

L₄₀₁ may be a ligand represented by Formula 402, and xc1 may be 1, 2, or 3, wherein, when xc1 is two or more, two or more of L₄₀₁(s) may be identical to or different from each other,

L₄₀₂ may be an organic ligand, and xc2 may be 0, 1, 2, 3, or 4, wherein, when xc2 is 2 or more, two or more of L₄₀₂(s) may be identical to or different from each other,

X₄₀₁ and X₄₀₂ may each independently be nitrogen or carbon,

ring A₄₀₁ and ring A₄₀₂ may each independently be a C₃-C₆₀ carbocyclic group or a C₁-C₆₀ heterocyclic group,

T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)=C(Q₄₁₂)-*′, *—C(Q₄₁₁)=*′, or *═C(Q₄₁₁)=*′,

X₄₀₃ and X₄₀₄ may each independently be a chemical bond (for example, a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),

Q₄₁₁ to Q₄₁₄ are respectively the same as those described in connection with Q₁,

R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),

Q₄₀₁ to Q₄₀₃ are respectively the same as those described in connection with Q₁,

xc11 and xc12 may each independently be an integer from 0 to 10, and

* and *′ in Formula 402 each indicate a binding site to M in Formula 401.

In an embodiment, in Formula 402, i) X₄₀₁ may be nitrogen, and X₄₀₂ may be carbon, or ii) each of X₄₀₁ and X₄₀₂ may be nitrogen.

In an embodiment, when xc1 in Formula 402 is 2 or more, two ring A₄₀₁ in two or more of L₄₀₁(s) may be optionally linked to each other via T₄₀₂, which is a linking group, and two ring A₄₀₂ may optionally be linked to each other via T₄₀₃, which is a linking group (see e.g., Compounds PD1 to PD4 and PD7). T₄₀₂ and T₄₀₃ are respectively the same as those described in connection with T₄₀₁.

L₄₀₂ in Formula 401 may be an organic ligand. In an embodiment, L₄₀₂ may include a halogen group, a diketone group (for example, an acetylacetonate group), a carboxylic acid group (for example, a picolinate group), —C(═O), an isonitrile group, —CN group, a phosphorus group (for example, a phosphine group, a phosphite group, etc.), or any suitable combinations thereof.

The phosphorescent dopant may include, for example, one of Compounds PD1 to PD39 or any suitable combinations thereof:

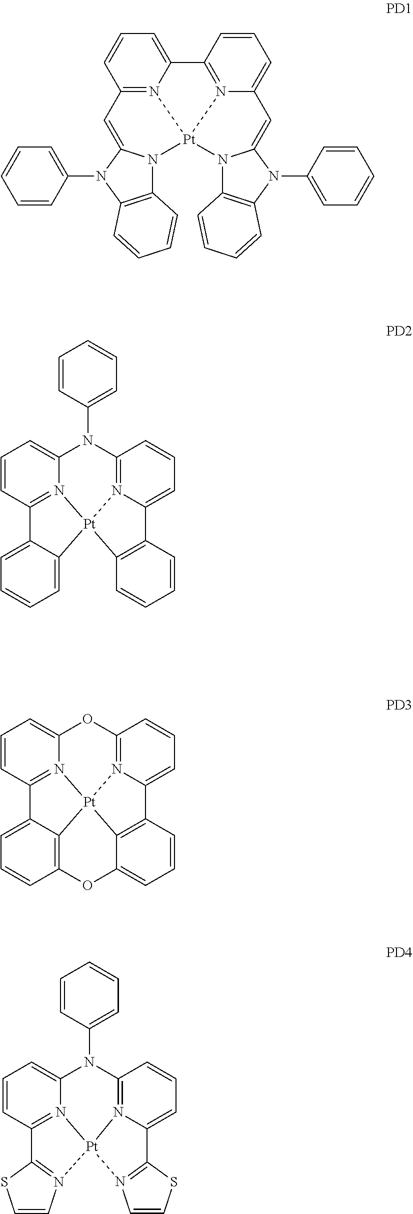

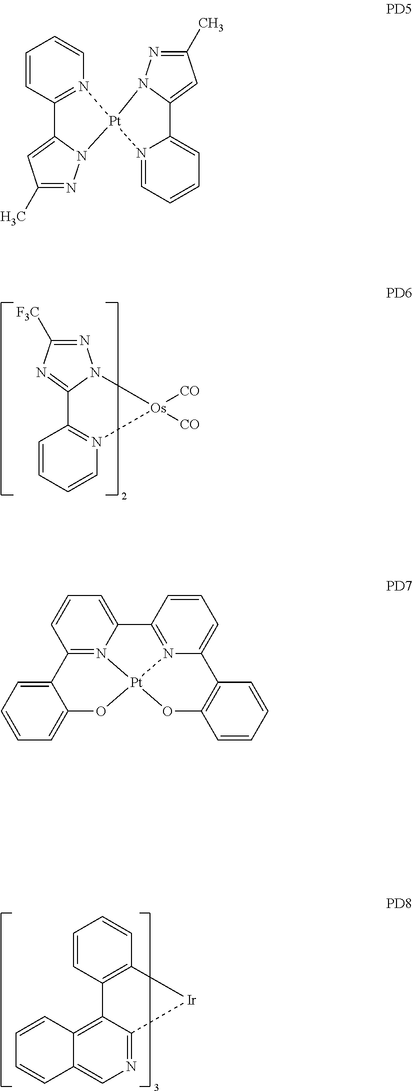

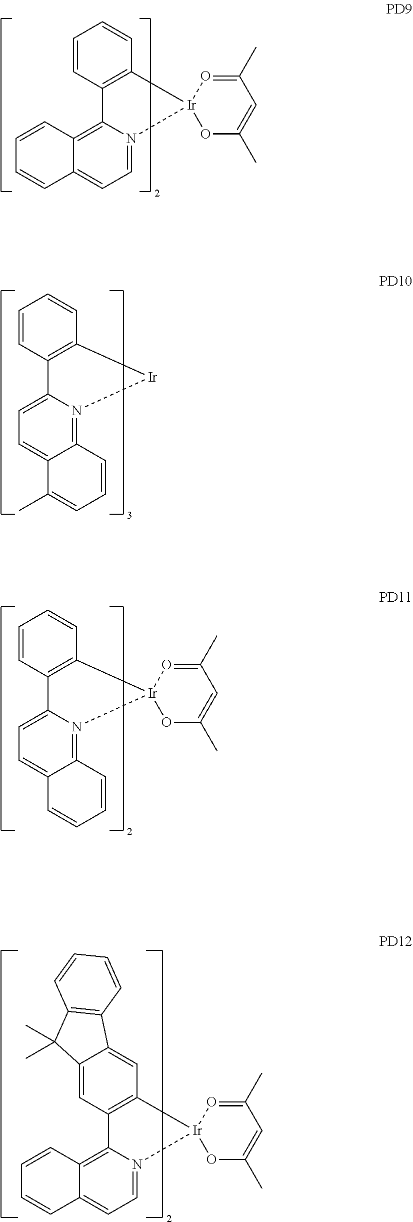

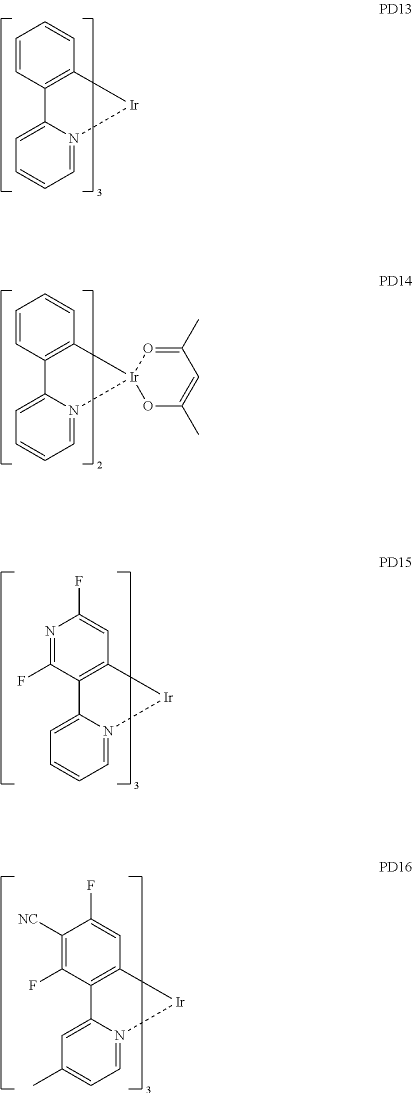

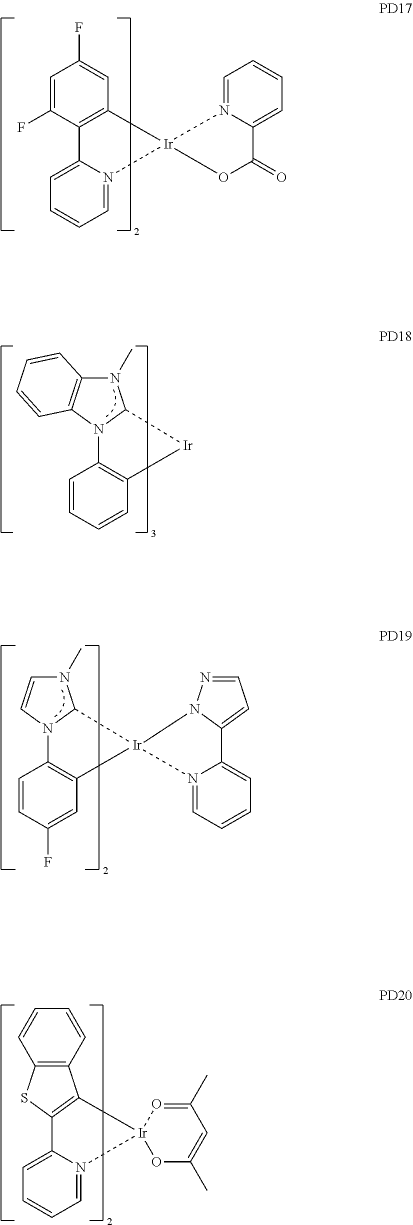

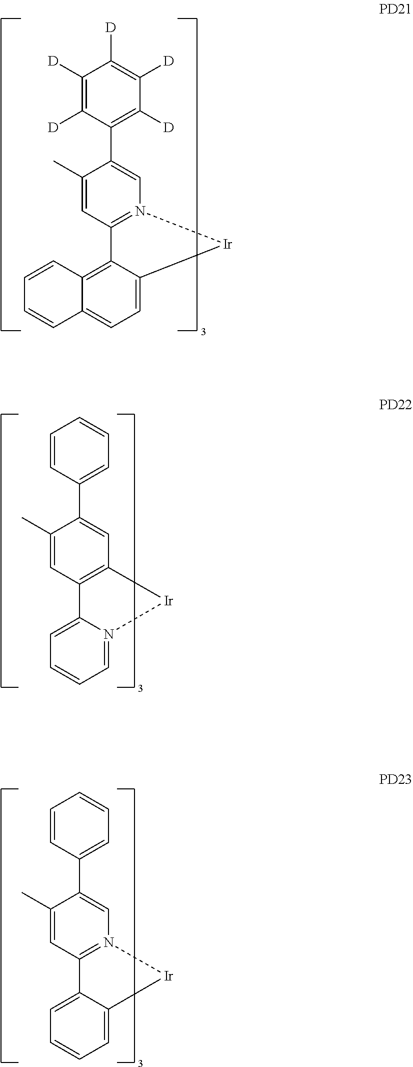

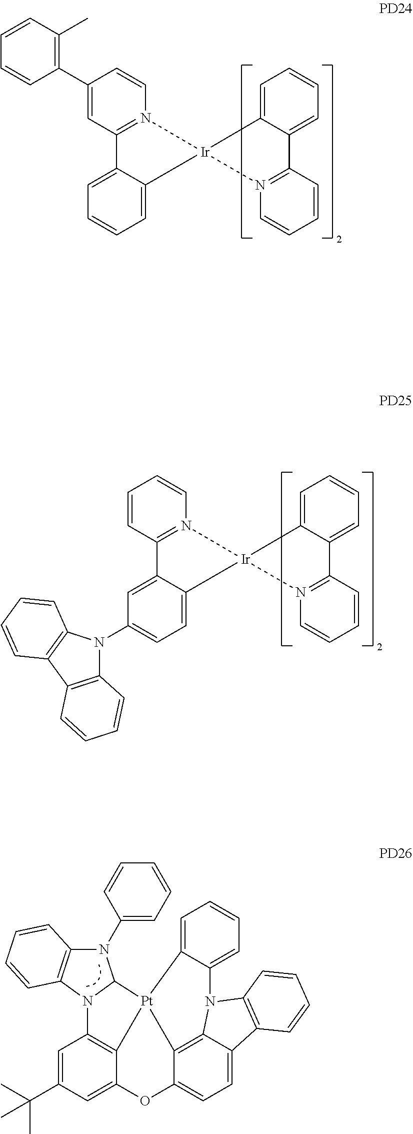

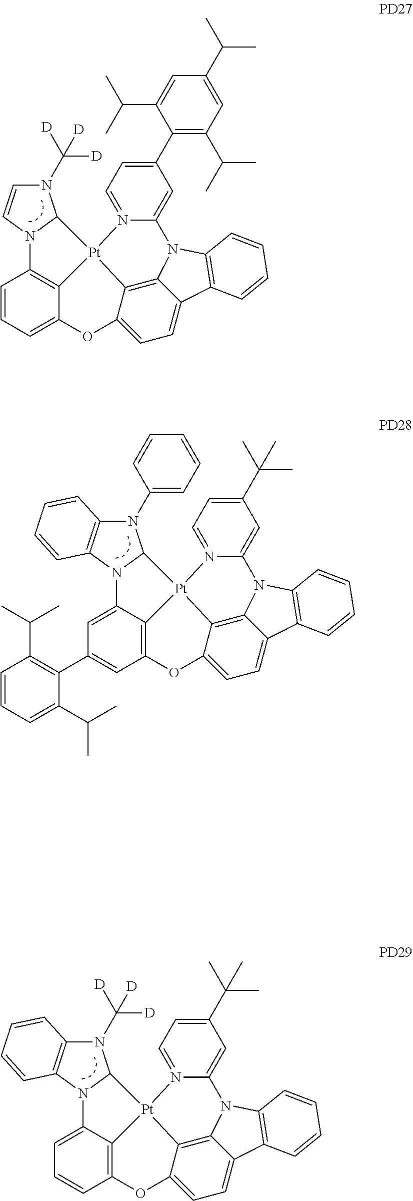

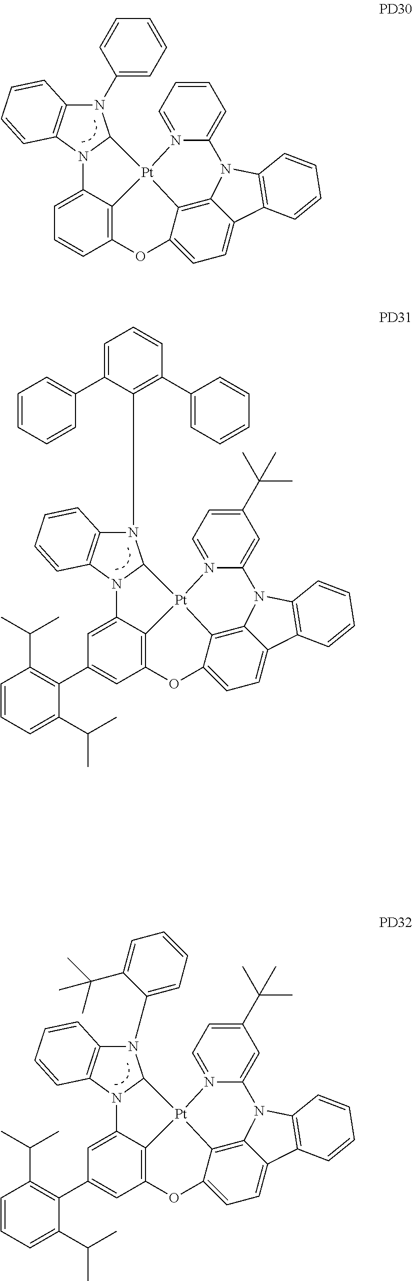

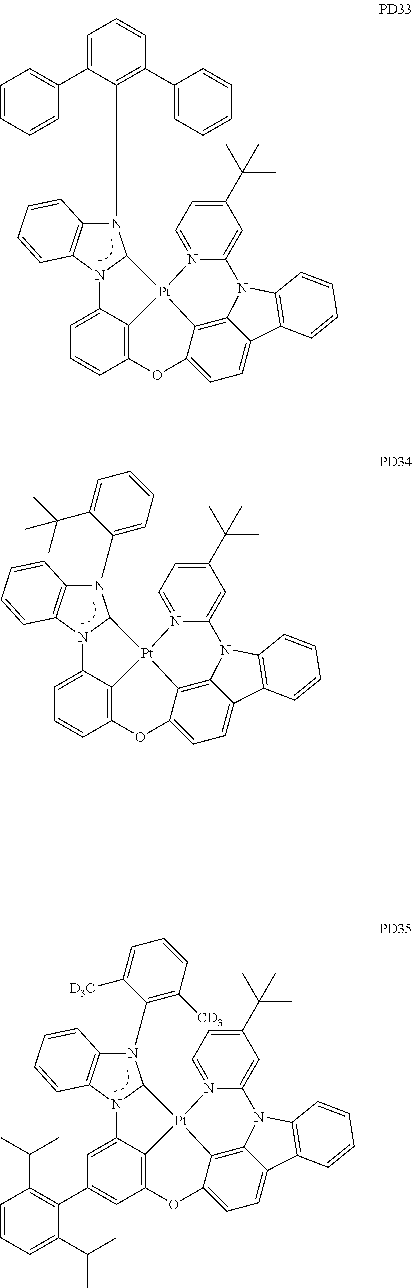

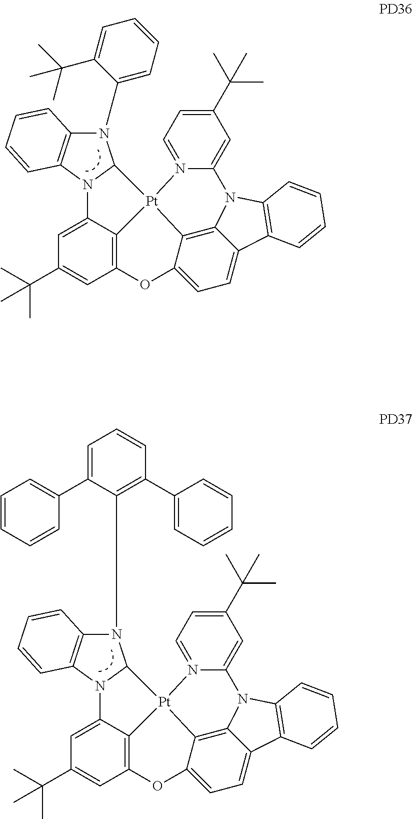

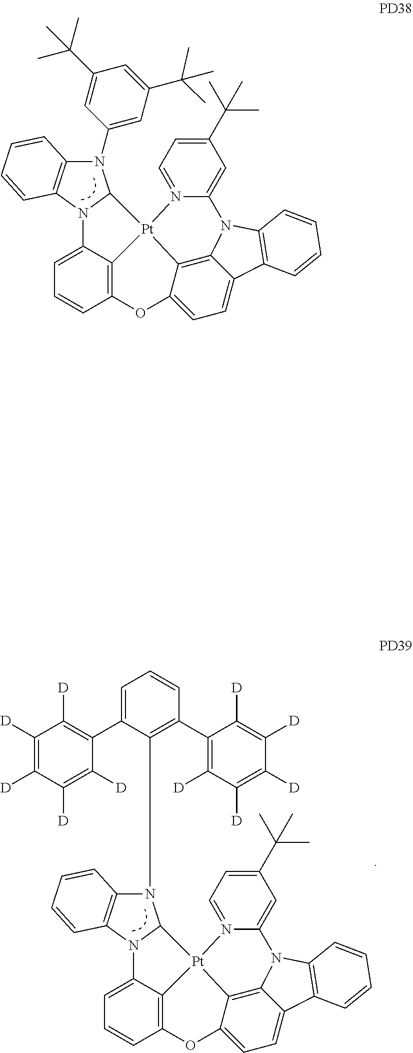

### Fluorescent Dopant

The fluorescent dopant may include an amine group-containing compound, a styryl group-containing compound, or any suitable combination thereof.

In an embodiment, the fluorescent dopant may include a compound represented by Formula 501:

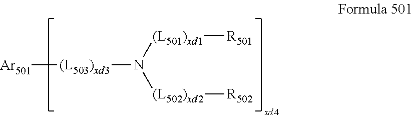

wherein, in Formula 501,

Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xd1 to xd3 may each independently be 0, 1, 2, or 3, and

xd4 may be 1, 2, 3, 4, 5, or 6.

In an embodiment, Ar₅₀₁ in Formula 501 may be a condensed cyclic group (for example, an anthracene group, a chrysene group, or a pyrene group) in which three or more monocyclic groups are condensed together.

In an embodiment, xd4 in Formula 501 may be 2.

In an embodiment, the fluorescent dopant may include one of Compounds FD1 to FD36, DPVBi, DPAVBi, or any suitable combinations thereof:

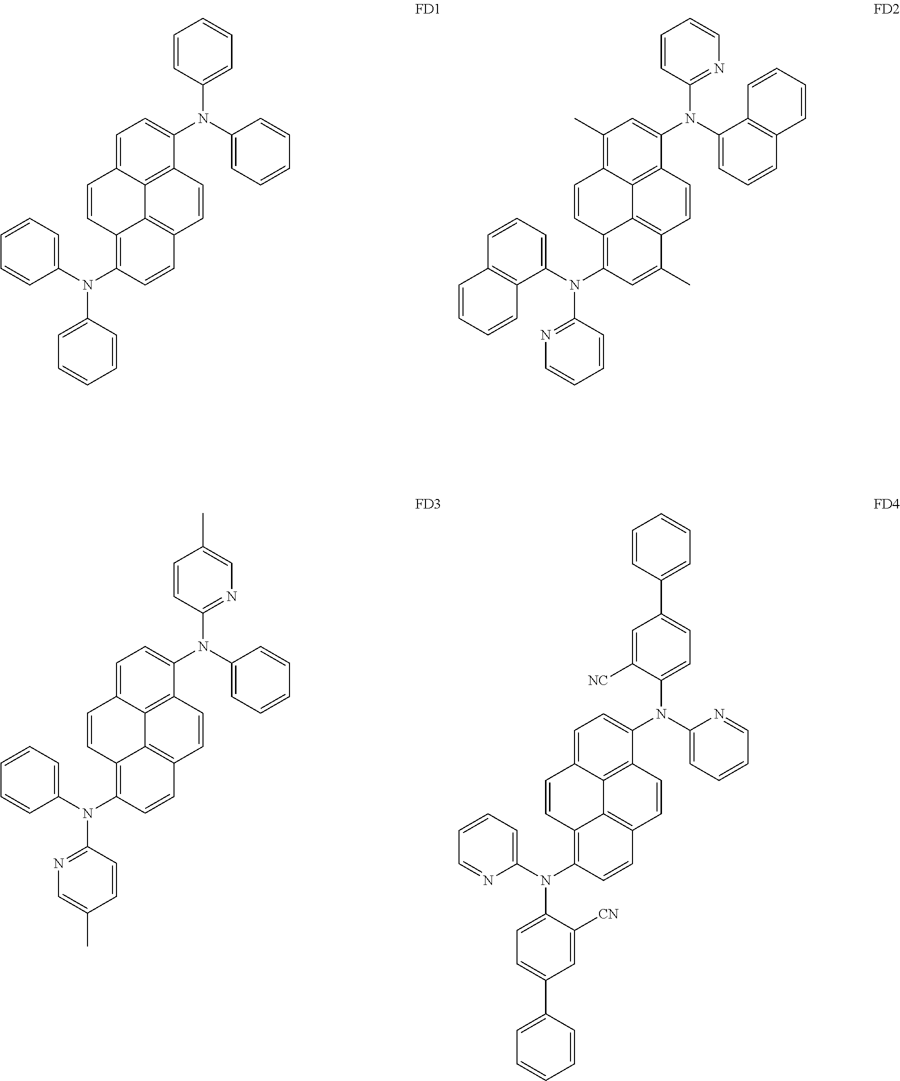 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1 ; N#Cc1cc(-c2ccccc2)ccc1N(c1ccccn1)c1ccc2ccc3c(N(c4ccccn4)c4ccc(-c5ccccc5)cc4C#N)ccc4ccc1c2c43]

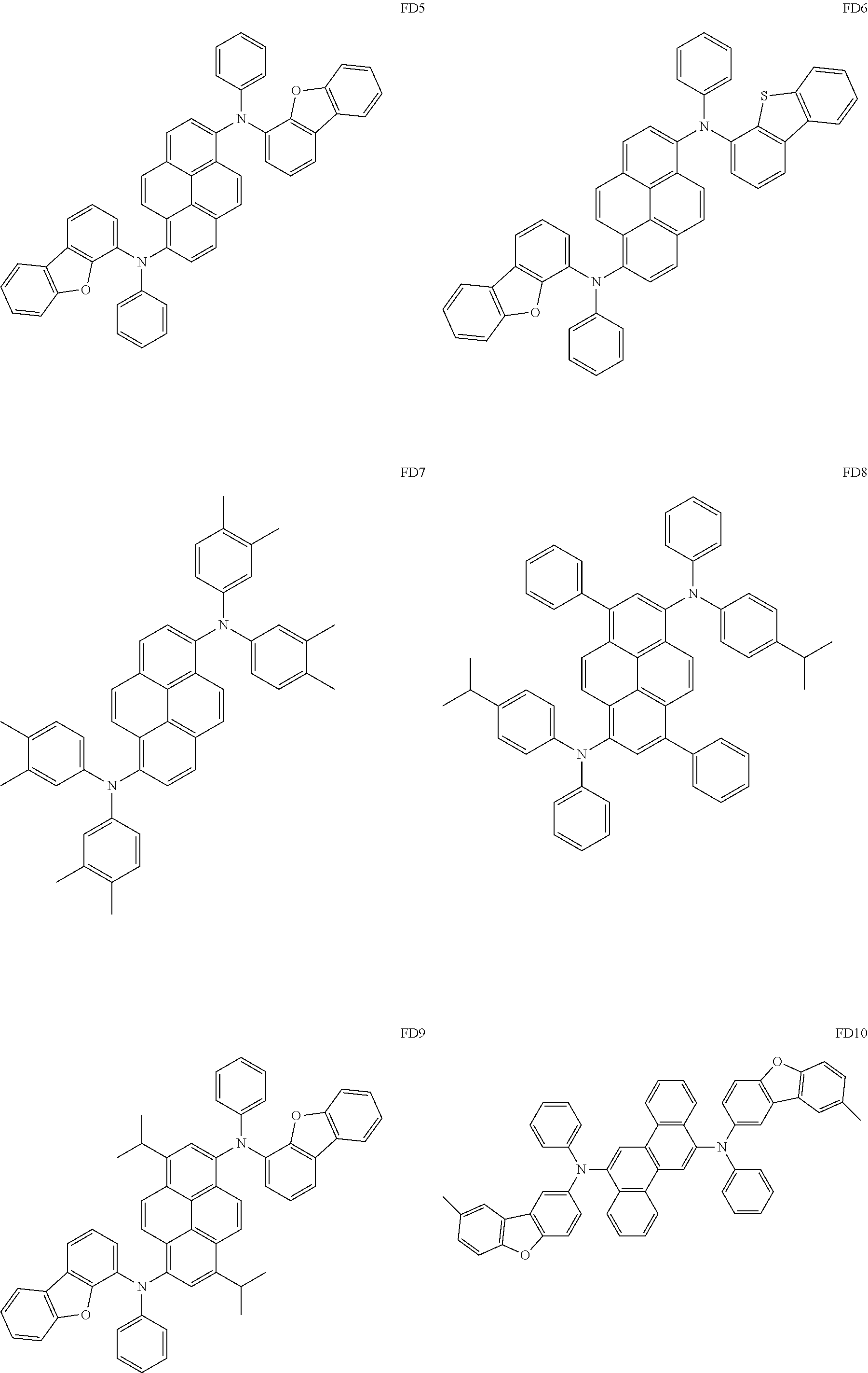

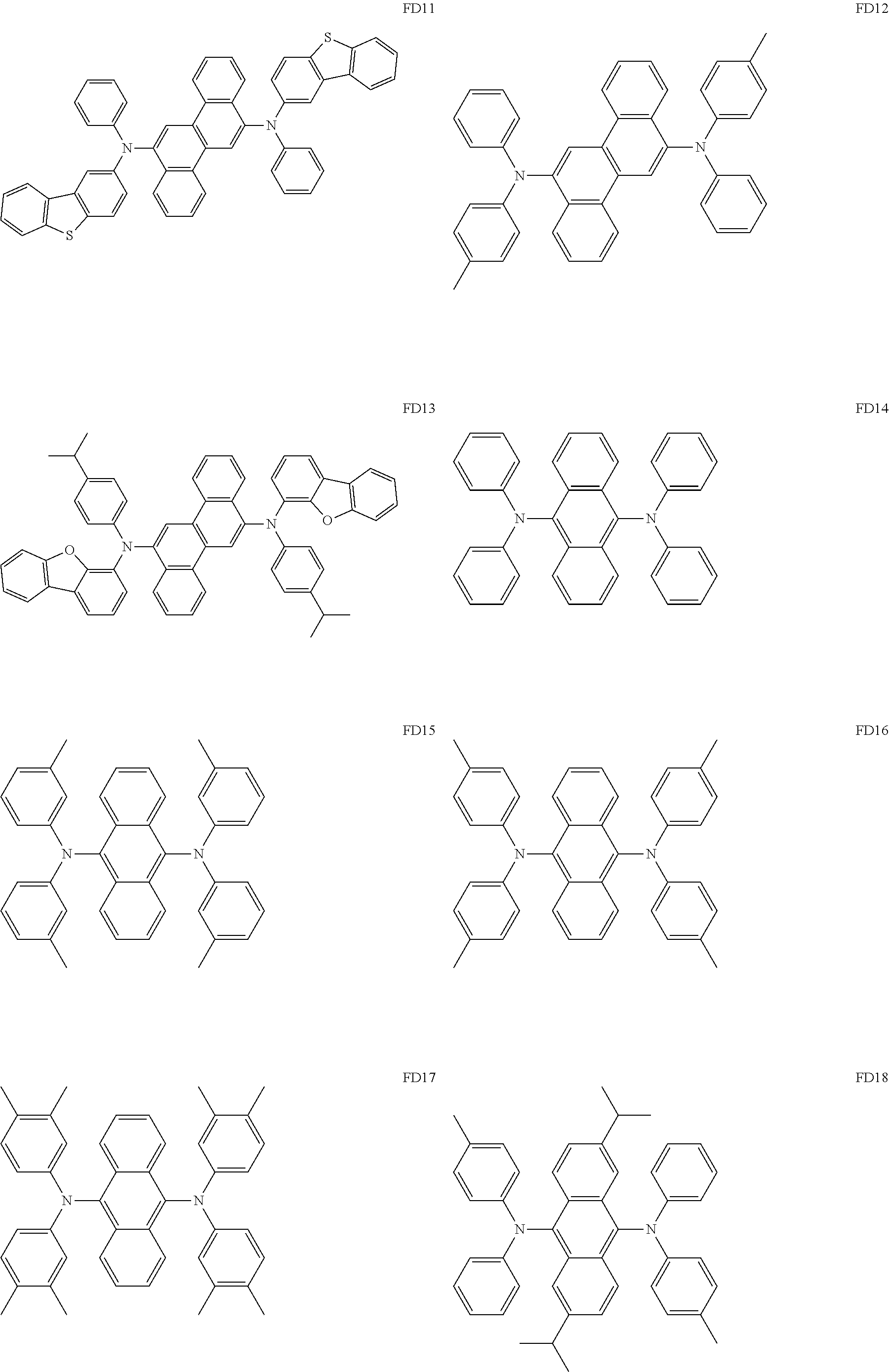

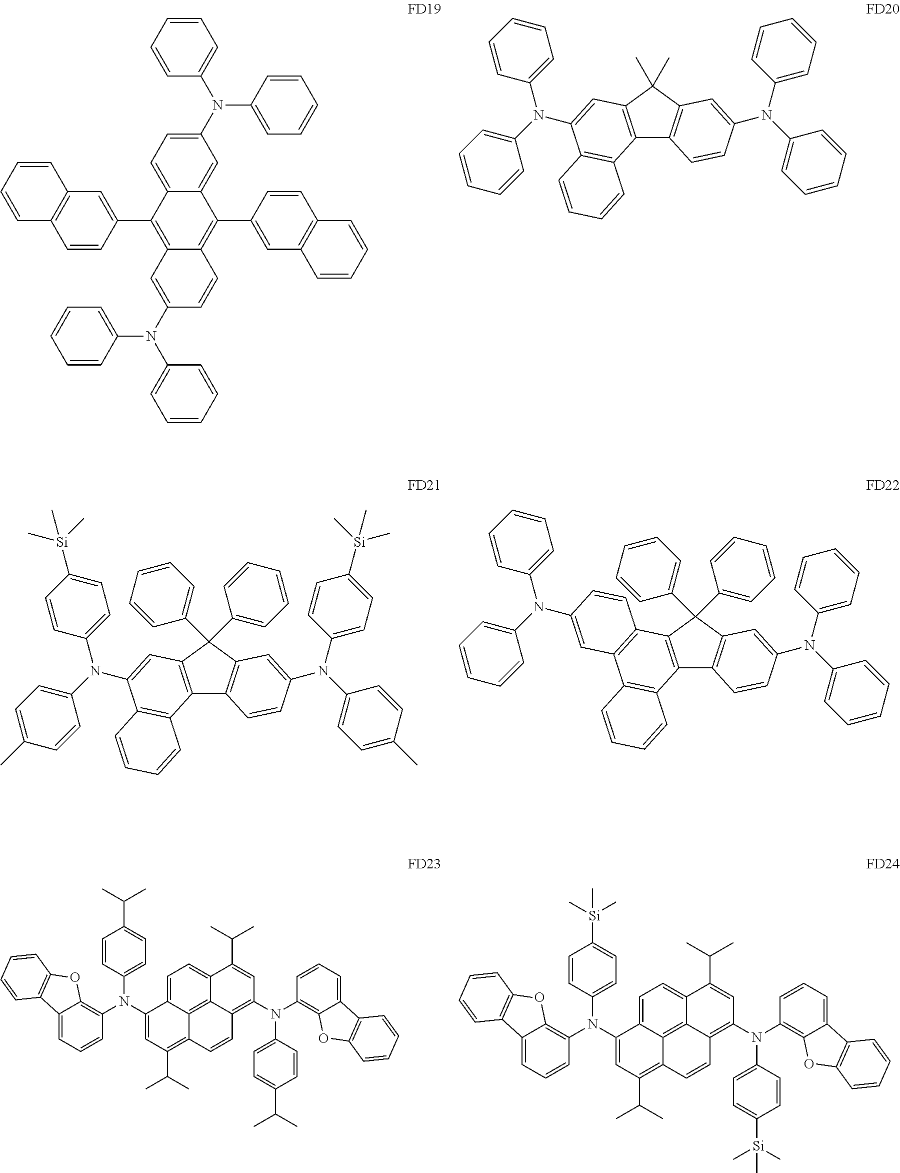

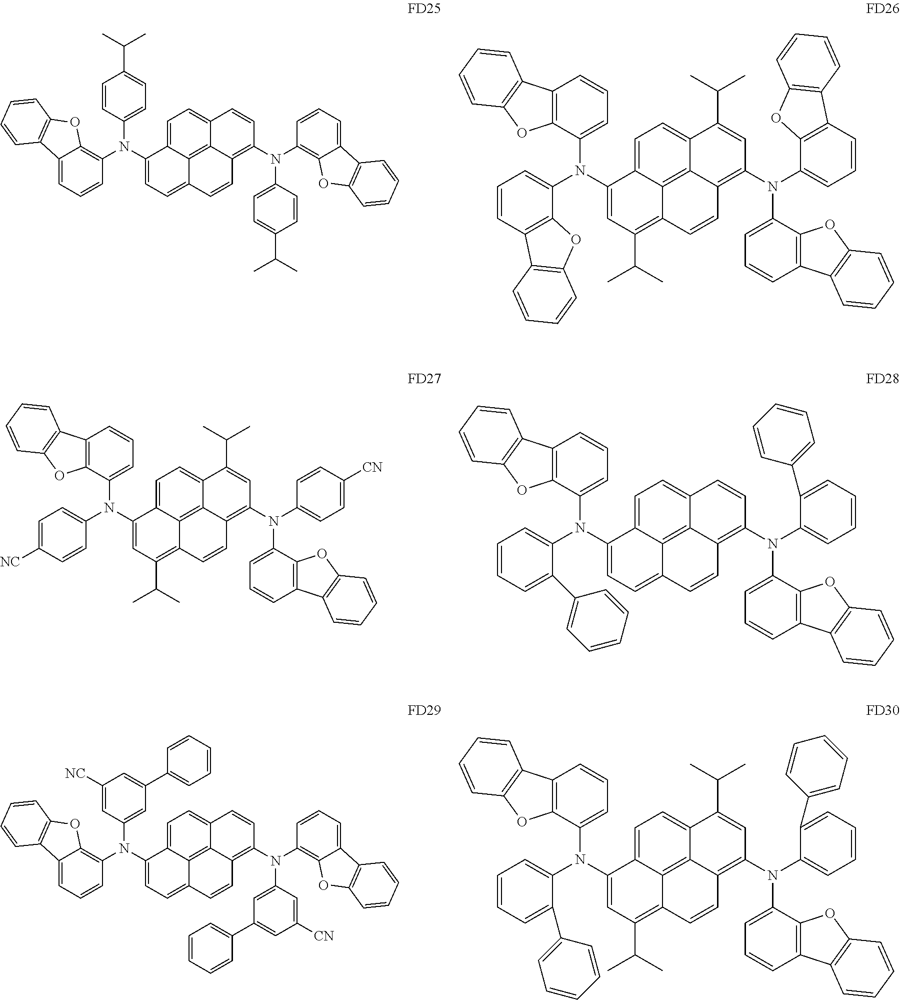

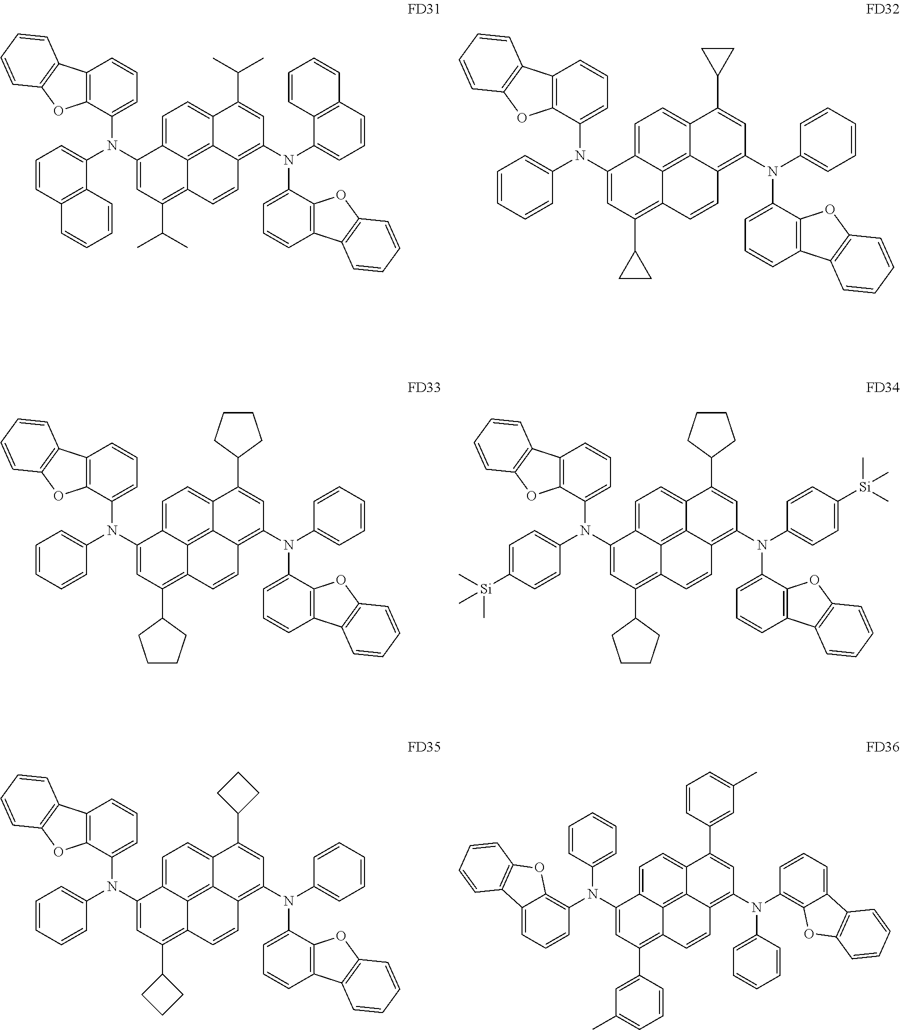

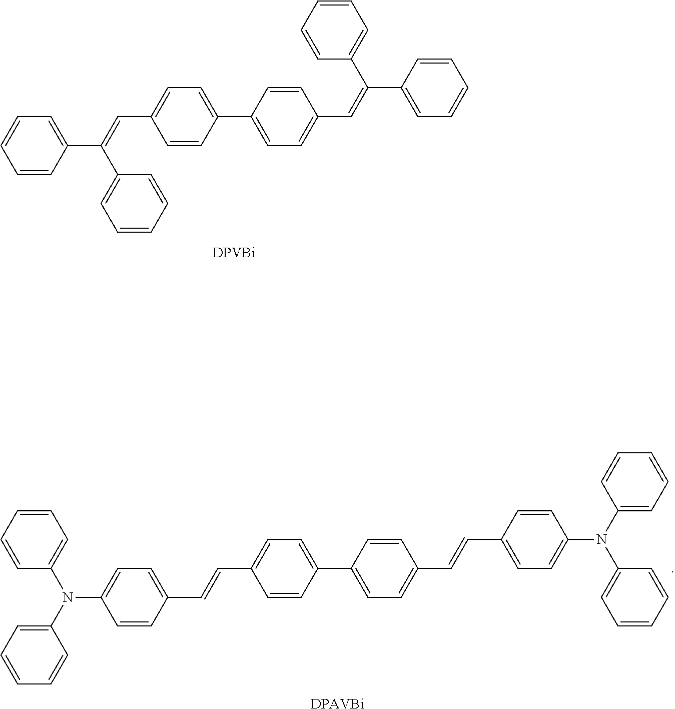

### Delayed Fluorescence Material

The emission layer may include a delayed fluorescence material.

In one or more embodiments of the present disclosure, the delayed fluorescence material may be selected from compounds capable of emitting delayed fluorescence based on a delayed fluorescence emission mechanism.

The delayed fluorescence material included in the emission layer may act as a host or a dopant depending on the type of other materials included in the emission layer.

In an embodiment, the difference between the triplet energy level (eV) of the delayed fluorescence material and the singlet energy level (eV) of the delayed fluorescence material may be greater than or equal to 0 eV and less than or equal to 0.5 eV. When the difference between the triplet energy level (eV) of the delayed fluorescence material and the singlet energy level (eV) of the delayed fluorescence material satisfies the above range, up-conversion from the triplet state to the singlet state of the delayed fluorescence materials may occur effectively, and thus, the luminescence efficiency of the light-emitting device 10 may be improved.

In an embodiment, the delayed fluorescence material may include i) a material including at least one electron donor (for example, a π electron-rich C₃-C₆₀ cyclic group, such as a carbazole group) and at least one electron acceptor (for example, a sulfoxide group, a cyano group, or a π electron-deficient nitrogen-containing C₁-C₆₀ cyclic group), and ii) a material including a C₈-C₆₀ polycyclic group in which two or more cyclic groups are condensed while sharing boron (B).

Examples of the delayed fluorescence material may include at least one of the following Compounds DF1 to DF9:

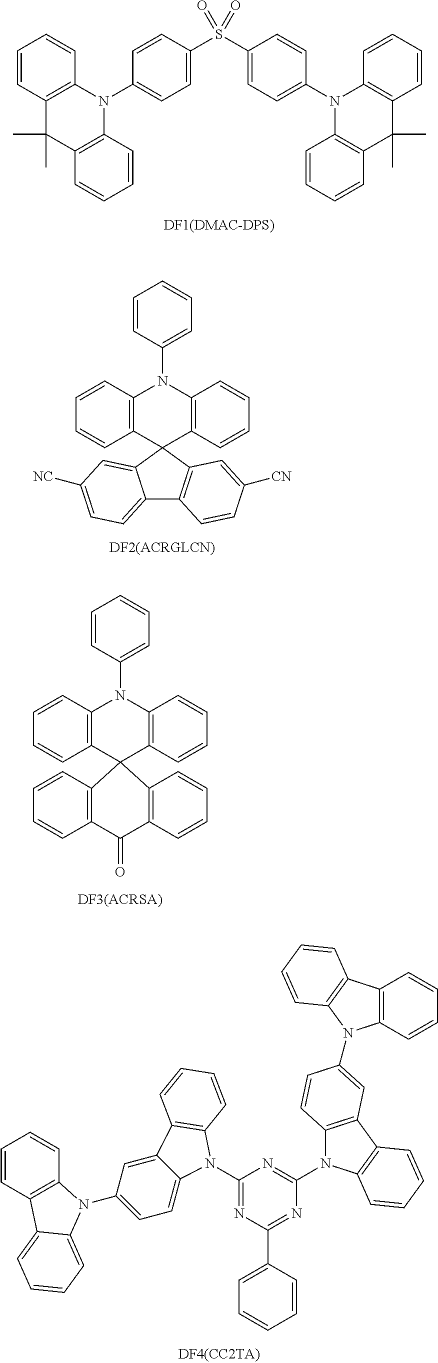

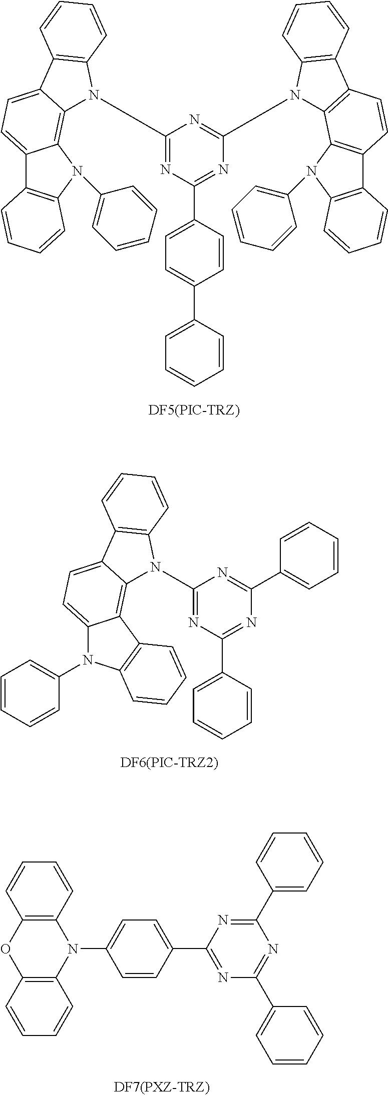

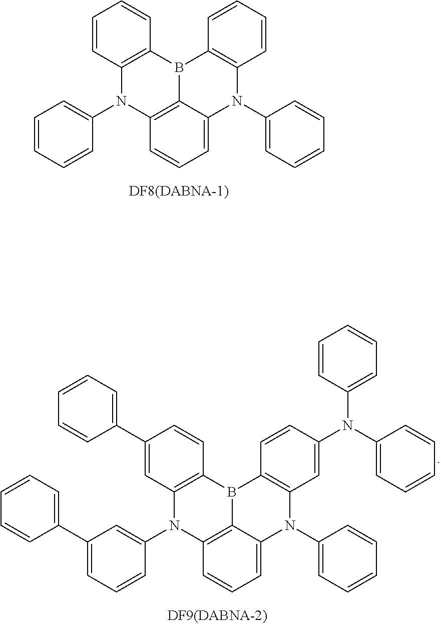 [SMILES: c1ccc(N2c3ccccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1]

### Quantum Dot

The emission layer may include a quantum dot.

In the present specification, a quantum dot refers to a crystal of a semiconductor compound, and may include any suitable material capable of emitting light of various suitable emission wavelengths according to the size of the crystal.

A diameter of the quantum dot may be, for example, in a range of about 1 nm to about 10 nm, or any range contained within.

The quantum dot may be synthesized by a wet chemical process, a metal organic chemical vapor deposition process, a molecular beam epitaxy process, or any suitable process similar thereto that should be apparent to one of ordinary skill in the art upon reviewing the disclosure.

According to the wet chemical process, a precursor material is mixed with an organic solvent to grow a quantum dot particle crystal. When the crystal grows, the organic solvent naturally acts as a dispersant coordinated on the surface of the quantum dot crystal and controls the growth of the crystal so that the growth of quantum dot particles can be controlled through a process which is more easily performed than vapor deposition methods, such as metal organic chemical vapor deposition (MOCVD) or molecular beam epitaxy (MBE), and which requires low costs.

The quantum dot may include: a Group II-VI semiconductor compound; a Group III-V semiconductor compound; a Group III-VI semiconductor compound; a Group I-III-VI semiconductor compound; a Group IV-VI semiconductor compound; a Group IV element or compound; or any combination thereof.

Examples of the Group II-VI semiconductor compound may include: a binary compound, such as CdSe, CdTe, ZnS, ZnSe, ZnTe, ZnO, HgS, HgSe, HgTe, MgSe, or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, or MgZnS; a quaternary compound, such as CdZnSeS, CdZnSeTe, CdZnSTe, CdHgSeS, CdHgSeTe, CdHgSTe, HgZnSeS, HgZnSeTe, or HgZnSTe; or any suitable combinations thereof.

Examples of the Group III-V semiconductor compound may include: a binary compound, such as GaN, GaP, GaAs, GaSb, AlN, AIP, AIAs, AISb, InN, InP, InAs, InSb, or the like; a ternary compound, such as GaNP, GaNAs, GaNSb, GaPAs, GaPSb, AINP, AINAs, AINSb, AIPAs, AIPSb, InGaP, InNP, InAIP, InNAs, InNSb, InPAs, InPSb, or the like; a quaternary compound, such as GaAINP, GaAINAs, GaAINSb, GaAIPAs, GaAIPSb, GaInNP, GaInNAs, GalnNSb, GaInPAs, GalnPSb, InAINP, InAINAs, InAINSb, InAIPAs, InAIPSb, and/or the like; or any suitable combinations thereof.

the Group III-V semiconductor compound may further include a Group II element. Examples of the Group III-V semiconductor compound further including Group II elements may include InZnP, InGaZnP, InAIZnP, and/or the like.

Examples of the Group III-VI semiconductor compound may include: a binary compound, such as GaS, GaSe, Ga₂Se₃, GaTe, InS, InSe, In₂S3, In₂Se₃, InTe, or the like; a ternary compound, such as InGaS₃, InGaSe₃, or the like; or any suitable combinations thereof.

Examples of the Group I-III-VI semiconductor compound may include: a ternary compound, such as AgInS, AgInS₂, CulnS, CuInS₂, CuGaO₂, AgGaO₂, or AgAlO₂; or any suitable combinations thereof.

Examples of the Group IV-VI semiconductor compound may include: a binary compound, such as SnS, SnSe, SnTe, PbS, PbSe, PbTe, or the like; a ternary compound, such as SnSeS, SnSeTe, SnSTe, PbSeS, PbSeTe, PbSTe, SnPbS, SnPbSe, SnPbTe, or the like; a quaternary compound, such as SnPbSSe, SnPbSeTe, SnPbSTe, and/or the like; or any suitable combinations thereof.

The Group IV element or compound may include: a single element compound, such as Si or Ge; a binary compound, such as SiC or SiGe; or any suitable combinations thereof.

Each element included in a multi-element compound such as the binary compound, ternary compound and quaternary compound, may exist in a particle form with a substantially uniform concentration or non-uniform concentration.

In an embodiment, the quantum dot may have a single structure or a dual core-shell structure. In an embodiment in which the quantum dot has a single structure, the concentration of each element included in the corresponding quantum dot is substantially uniform. In an embodiment, the material contained in the core and the material contained in the shell may be different from each other.

The shell of the quantum dot may act as a protective layer to prevent (reduce) chemical degeneration of the core to maintain semiconductor characteristics and/or as a charging layer to impart electrophoretic characteristics to the quantum dot. The shell may be a single layer or a multi-layer. The element presented in the interface between the core and the shell of the quantum dot may have a concentration gradient that decreases toward the center of the quantum dot.

Examples of the shell of the quantum dot may be an oxide of metal, metalloid, or non-metal, a semiconductor compound, or combinations thereof. Examples of the oxides of metal, metalloid, or non-metal may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; a ternary compound, such as MgAl₂O₄, CoFe204, NiFe₂O₄, or CoMn₂O₄; or any suitable combinations thereof. Examples of the semiconductor compound may include, as described herein, a Group II-VI semiconductor compound, a Group III-V semiconductor compound, a Group III-VI semiconductor compound, a Group I-III-VI semiconductor compound, a Group IV-VI semiconductor compound, or any combination thereof. In addition, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AIAs, AIP, AISb, or any suitable combinations thereof.

A full width at half maximum (FWHM) of an emission wavelength spectrum of the quantum dot may be about 45 nm or less, for example, about 40 nm or less, for example, about 30 nm or less, and within these ranges, color purity or color reproducibility may be increased.

In addition, because the light emitted through the quantum dot is emitted in all directions, the wide viewing angle can be improved (increased).

In addition, the quantum dot may be a substantially spherical particle, a pyramidal particle, a multi-arm particle, a cubic nanoparticle, a nanotube particle, a nanowire particle, a nanofiber particle, or a nanoplate particle.

Because the energy band gap can be adjusted by controlling the size of the quantum dot, light having various suitable wavelength bands may be obtained from the quantum dot emission layer. Therefore, by using quantum dots of different sizes, a light-emitting device that emits light of various wavelengths may be implemented. In an embodiment, the size of the quantum dot may be selected to emit red, green and/or blue light. In addition, the size of the quantum dot may be configured to emit white light by combining light of various suitable colors.

### Electron Transport Region in Interlayer 130

The electron transport region may have: i) a single-layered structure including a single layer including a single material, ii) a single-layered structure including a single layer including a plurality of different materials, or iii) a multi-layered structure including a plurality of layers including different materials.

The electron transport region may include a buffer layer, a hole blocking layer, an electron control layer, an electron transport layer, an electron injection layer, or any suitable combination thereof.

For example, the electron transport region may have a structure including an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein, in each structure, layers are sequentially stacked from the emission layer.

The electron transport region (for example, the buffer layer, the hole blocking layer, the electron control layer, or the electron transport layer in the electron transport region) may include a metal-free compound including at least one π electron-deficient nitrogen-containing C₁-C₆₀ cyclic group.

In an embodiment, the electron transport region may include a compound represented by Formula 601 below:

[Ar₆₀₁]ₓₑ₁₁-[(L₆₀₁)ₓₑ₁-R₆₀₁]ₓₑ₂₁ Formula 601

wherein, in Formula 601,

Ar₆₀₁, and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ are respectively the same as those described in connection with Q₁,

xe21 may be 1, 2, 3, 4, or 5, and

at least one of Ar₆₀₁, L₆₀₁, or R₆₀₁ may each independently be a π electron-deficient nitrogen-containing C₁-C₆₀ cyclic group unsubstituted or substituted with at least one R₁₀ₐ.

In an embodiment, when xe11 in Formula 601 is 2 or more, two or more of Ar₆₀₁(s) may be linked via a single bond.

In an embodiment, Ar₆₀₁ in Formula 601 may be a substituted or unsubstituted anthracene group.

In an embodiment, the electron transport region may include a compound represented by Formula 601-1:

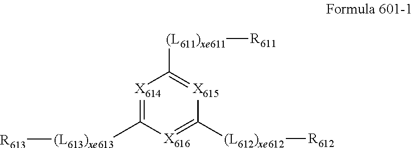

wherein, in Formula 601-1,

X₆₁₄ may be N or C(R₆₁₄), X₆₁₅ may be N or C(R₆₁₅), X₆₁₆ may be N or

C(R₆₁₆), and at least one of X₆₁₄ to X₆₁₆ may be N,

L₆₁₁ to L₆₁₃ are respectively the same as those described in connection with L₆₀₁,

xe611 to xe613 are respectively the same as those described in connection with xe1,

R₆₁₁ to R₆₁₃ are respectively the same as those described in connection with R₆₀₁, and

R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include one of Compounds ET1 to ET45, 2,9-dimethyl-4,7-diphenyl-1,10-phenanthroline (BCP), 4,7-diphenyl-1,10-phenanthroline (Bphen), Alq3, BAlq, TAZ, NTAZ, or any suitable combinations thereof:

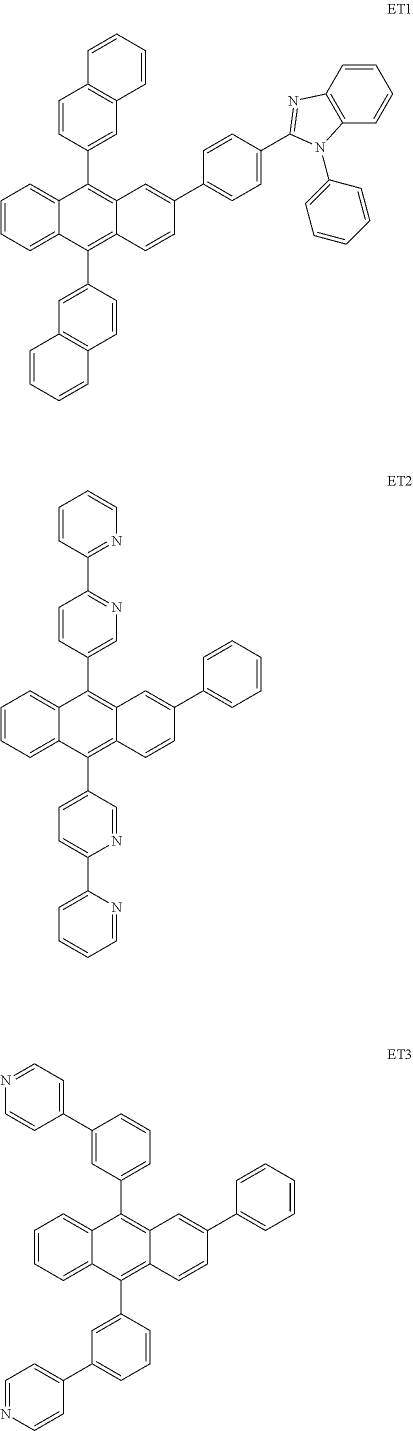 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

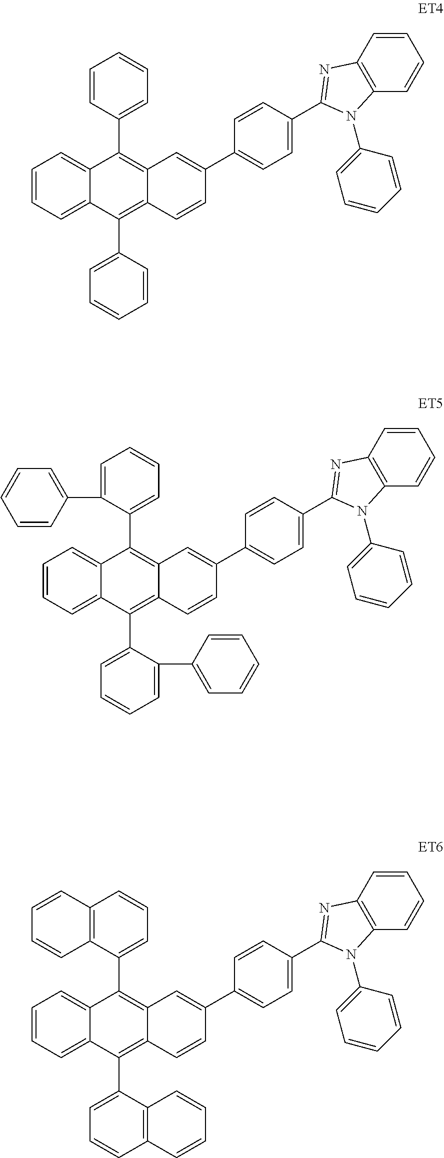 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

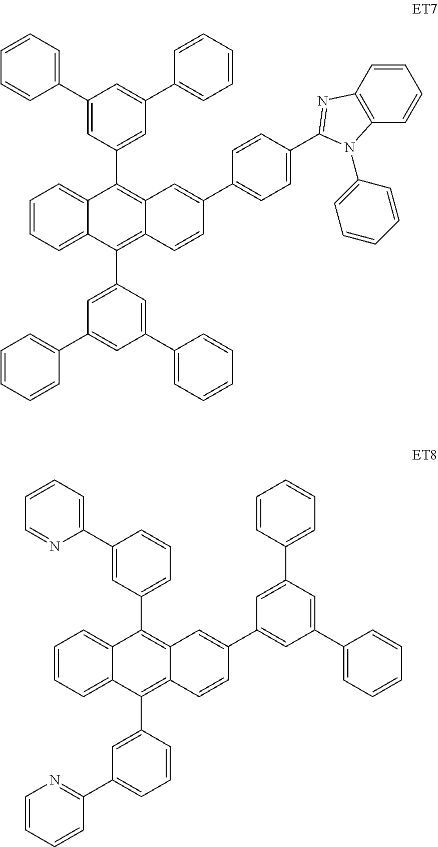 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1]

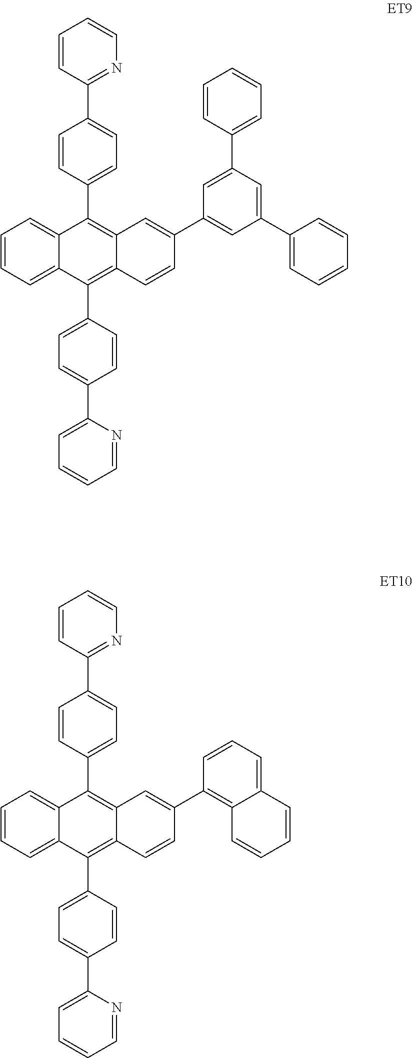 [SMILES: c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

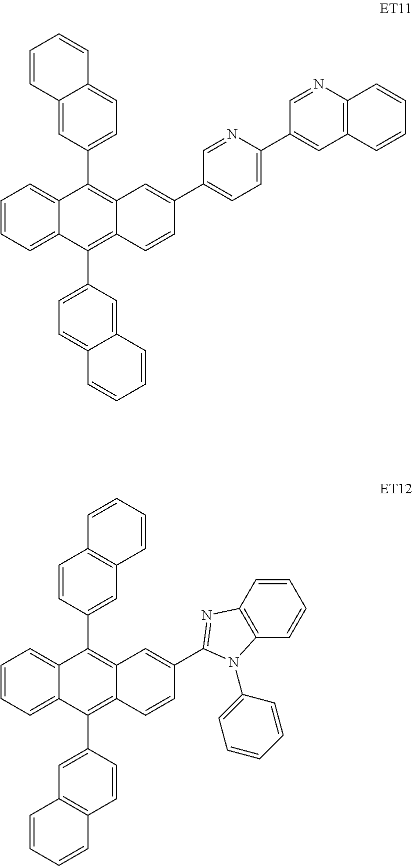 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

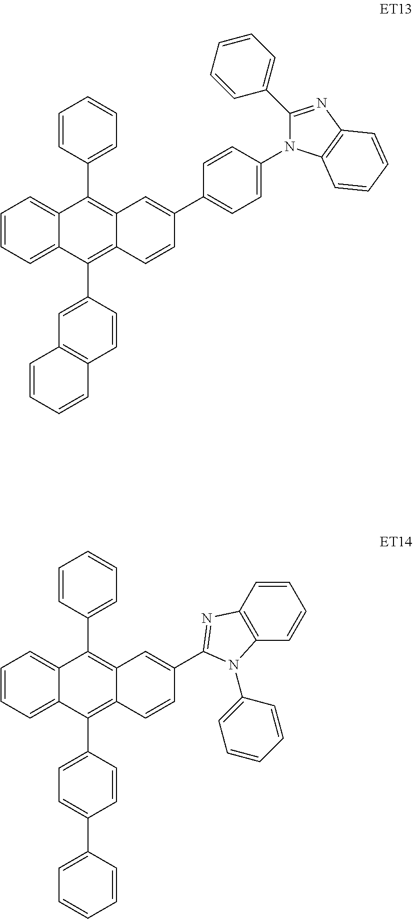 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1]

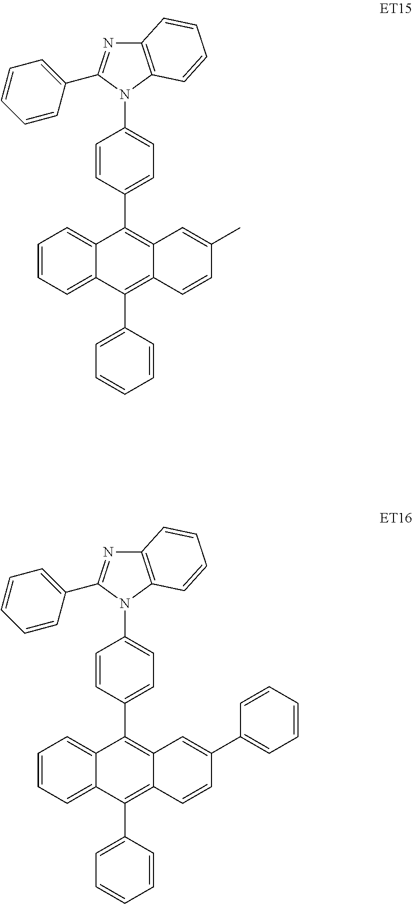 [SMILES: Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

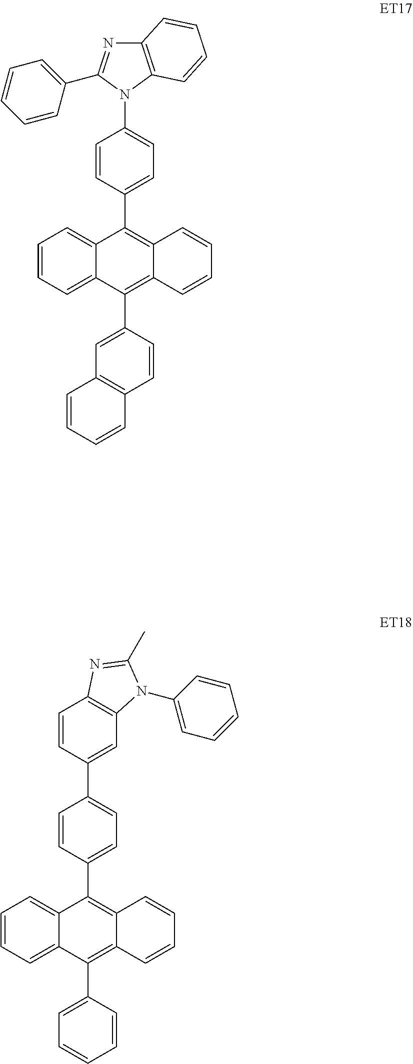 [SMILES: c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

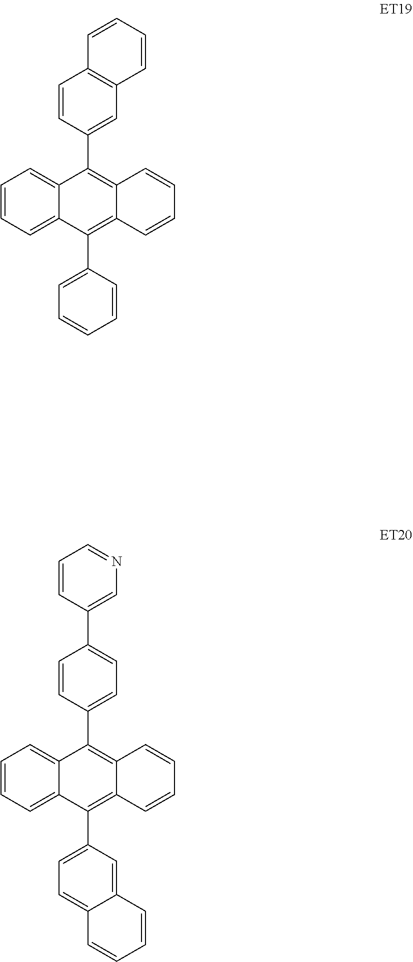

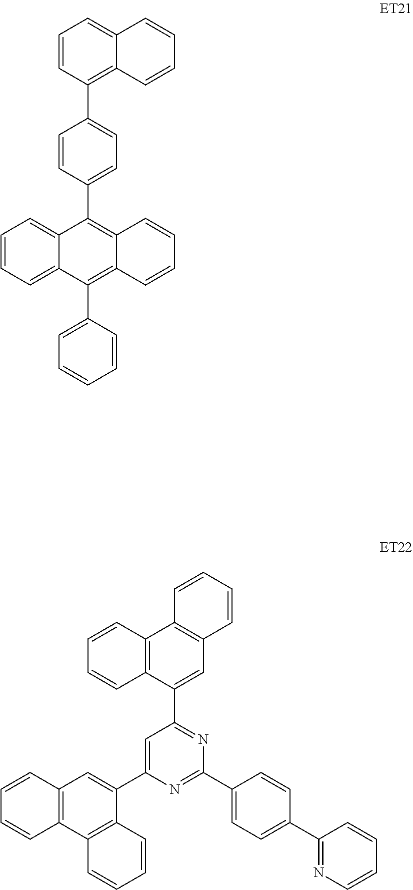

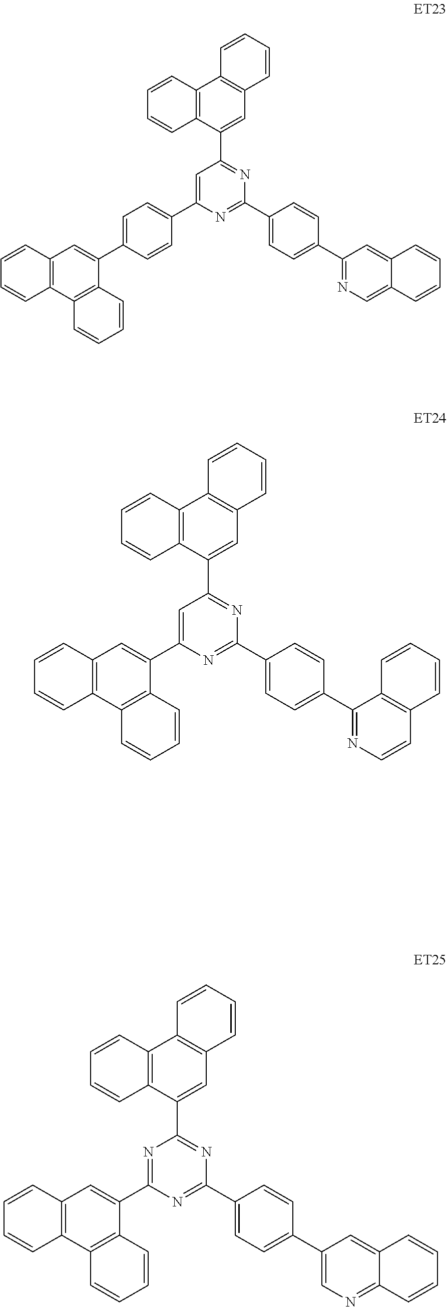 [SMILES: c1ccc2cc(-c3ccc(-c4nc(-c5ccc(-c6cc7ccccc7c7ccccc67)cc5)cc(-c5cc6ccccc6c6ccccc56)n4)cc3)ncc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

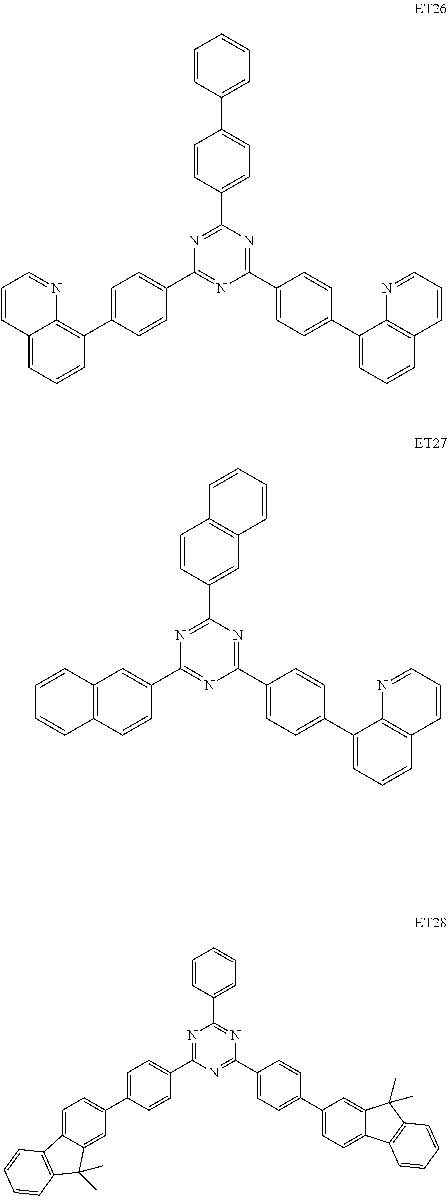 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

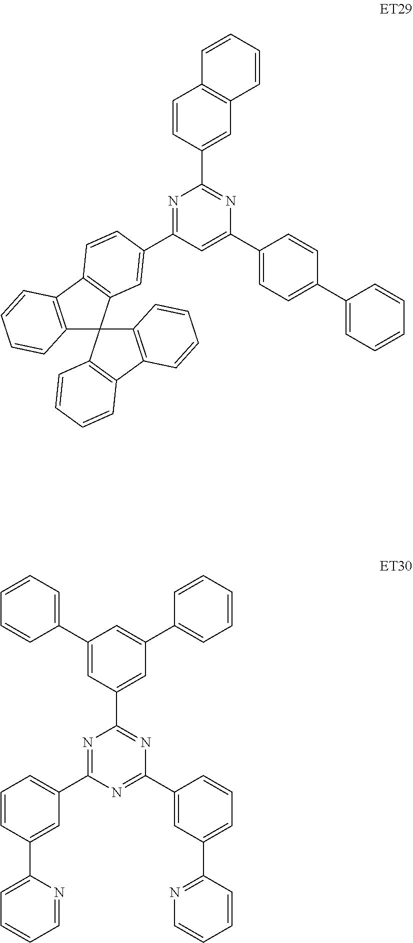 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

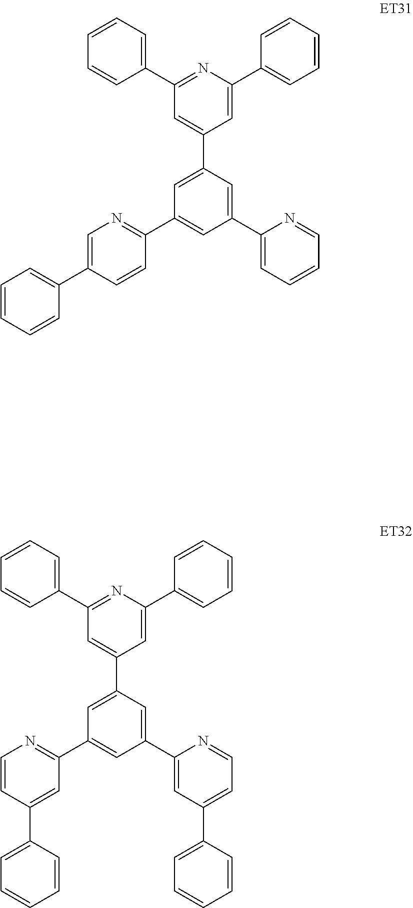 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

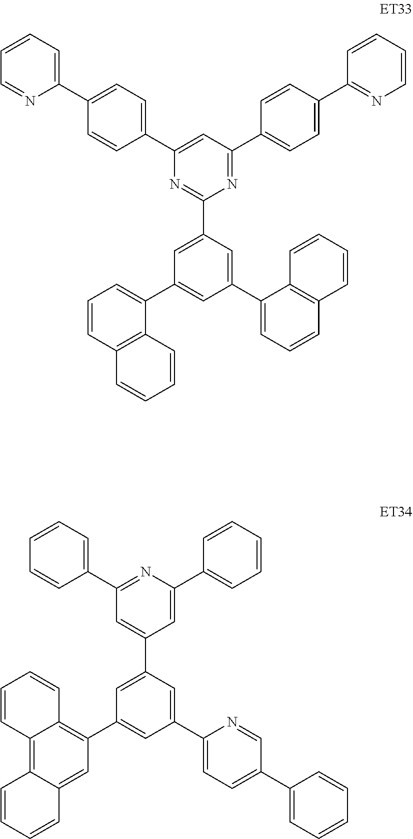 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

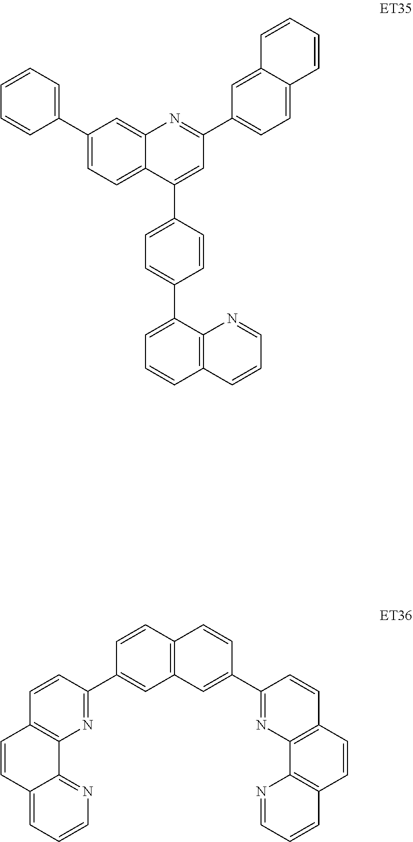 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

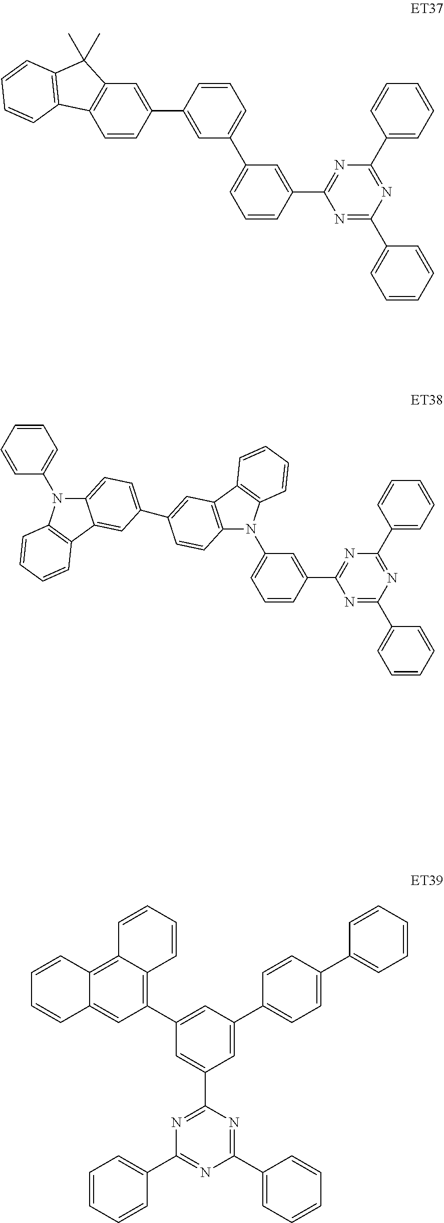 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1]

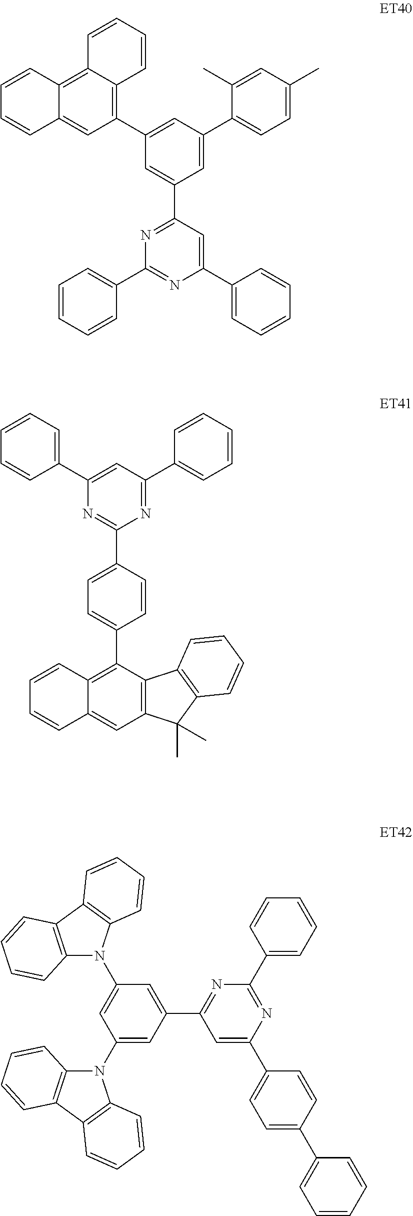 [SMILES: Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

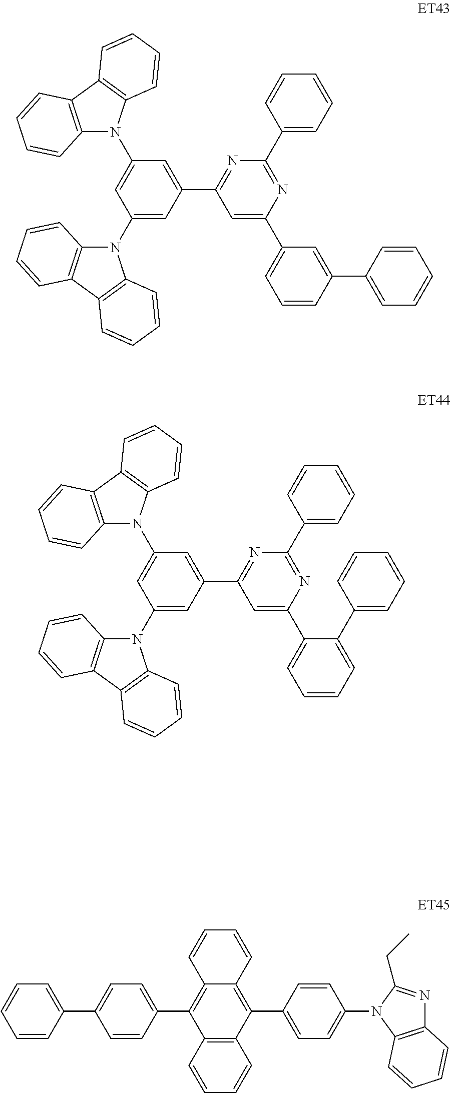 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

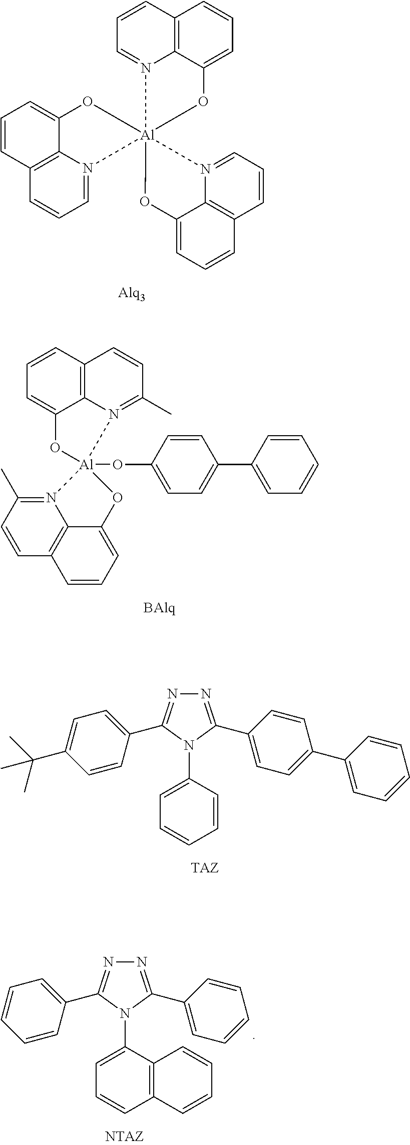

A thickness of the electron transport region may be in a range of about 100 Å to about 5,000 Å, for example, about 160 Å to about 4,000 Å. When the electron transport region includes a buffer layer, a hole blocking layer, an electron control layer, an electron transport layer, or any suitable combination thereof, a thickness of the buffer layer, the hole blocking layer, or the electron control layer may each independently be from about 20 Å to about 1,000 Å, for example, about 30 Å to about 300 Å, and a thickness of the electron transport layer may be from about 100 Å to about 1,000 Å, for example, about 150 Å to about 500 Å. When the thickness of the buffer layer, the hole blocking layer, the electron control layer, the electron transport layer, and/or the electron transport region are within these ranges, satisfactory (suitable) electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

In an embodiment, the metal-containing material may include a Li complex. The Li complex may include, for example, Compound ET-D1 (LiQ) or ET-D2:

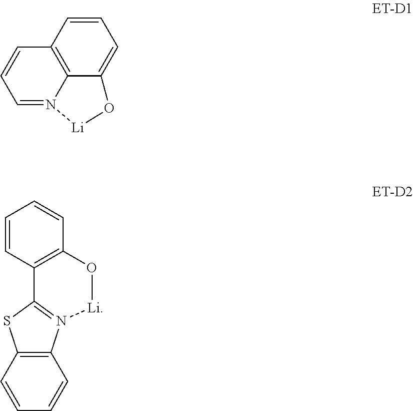

The electron transport region may include an electron injection layer that facilitates the injection of electrons from the second electrode 150. The electron injection layer may be in direct contact with the second electrode 150.

The electron injection layer may have: i) a single-layered structure including a single layer including a single material, ii) a single-layered structure including a single layer consisting of a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any suitable combinations thereof.

The alkali metal may include Li, Na, K, Rb, Cs, or any suitable combinations thereof. The alkaline earth metal may include Mg, Ca, Sr, Ba, or any combination thereof. The rare earth metal may include Sc, Y, Ce, Tb, Yb, Gd, or any suitable combinations thereof.

The alkali metal-containing compound, the alkaline earth metal-containing compound, and the rare earth metal-containing compound may include oxides, halides (for example, fluorides, chlorides, bromides, or iodides), or tellurides of the alkali metal, the alkaline earth metal, and the rare earth metal, or any suitable combinations thereof.

The alkali metal-containing compound may include alkali metal oxides, such as Li₂O, Cs₂O, or K₂O, alkali metal halides, such as LiF, NaF, CsF, KF, Lil, NaI, CsI, or KI, or any suitable combinations thereof. The alkaline earth metal-containing compound may include an alkaline earth metal compound, such as BaO, SrO, CaO, BaxSr₁₋ₓO (x is a real number satisfying the condition of 0<x<1), BaxCa₁₋ₓO (x is a real number satisfying the condition of 0<x<1), or the like. The rare earth metal-containing compound may include YbF₃, ScF₃, Sc₂O₃, Y₂O₃, Ce₂O₃, GdF₃, TbF₃, Ybl₃, ScI3, TbI₃, or any suitable combinations thereof. In an embodiment, the rare earth metal-containing compound may include lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and/or Lu₂Te₃.

The alkali metal complex, the alkaline earth-metal complex, and the rare earth metal complex may include i) one or more of ions of the alkali metal, the alkaline earth metal, or the rare earth metal and ii), as a ligand bonded to the metal ion, for example, a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenyl benzimidazole, a hydroxyphenylbenzothiazole, a bipyridine, a phenanthroline, a cyclopentadiene, or any suitable combinations thereof.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any suitable combinations thereof, as described above. In an embodiment, the electron injection layer may further include an organic material (for example, a compound represented by Formula 601).

In an embodiment, the electron injection layer may include i) an alkali metal-containing compound (for example, an alkali metal halide), ii) a) an alkali metal-containing compound (for example, an alkali metal halide); and b) an alkali metal, an alkaline earth metal, a rare earth metal, or any suitable combinations thereof. In an embodiment, the electron injection layer may be a KI:Yb co-deposited layer, an RbI:Yb co-deposited layer, and/or the like.

When the electron injection layer further includes an organic material, alkali metal, alkaline earth metal, rare earth metal, an alkali metal-containing compound, an alkaline earth metal-containing compound, a rare earth metal-containing compound, alkali metal complex, alkaline earth-metal complex, rare earth metal complex, or any suitable combinations thereof may be substantially homogeneously or non-homogeneously dispersed in a matrix that includes the organic material.

A thickness of the electron injection layer may be in a range of about 1 Å to about 100 Å, and, for example, about 3 Å to about 90 Å. When the thickness of the electron injection layer is within the range described above, satisfactory (suitable) electron injection characteristics may be obtained without a substantial increase in driving voltage.

### Second Electrode 150

The second electrode 150 may be located on the interlayer 130 having such a structure. The second electrode 150 may be a cathode, which is an electron injection electrode, and as the material for the second electrode 150, a metal, an alloy, an electrically conductive compound, or any combination thereof, each having a low work function, may be used.

In an embodiment, the second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or combinations thereof. The second electrode 150 may be a transmissive electrode, a semi-transmissive electrode, or a reflective electrode.

The second electrode 150 may have a single-layered structure or a multi-layered structure including two or more layers.

### Capping Layer

A first capping layer may be located outside the first electrode 110, and/or a second capping layer may be located outside the second electrode 150. In more detail, the light-emitting device 10 may have a structure in which the first capping layer, the first electrode 110, the interlayer 130, and the second electrode 150 are sequentially stacked in this stated order, a structure in which the first electrode 110, the interlayer 130, the second electrode 150, and the second capping layer are sequentially stacked in this stated order, or a structure in which the first capping layer, the first electrode 110, the interlayer 130, the second electrode 150, and the second capping layer are sequentially stacked in this stated order.

Light generated in an emission layer of the interlayer 130 of the light-emitting device 10 may be extracted toward the outside through the first electrode 110, which is a semi-transmissive electrode or a transmissive electrode, and the first capping layer or light generated in an emission layer of the interlayer 130 of the light-emitting device 10 may be extracted toward the outside through the second electrode 150, which is a semi-transmissive electrode or a transmissive electrode, and the second capping layer.

The first capping layer and the second capping layer may increase external emission efficiency according to the principle of constructive interference. Accordingly, the light extraction efficiency of the light-emitting device 10 is increased, so that the luminescence efficiency of the light-emitting device 10 may be improved.

Each of the first capping layer and second capping layer may include a material having a refractive index (at 589 nm) of 1.6 or more.

The first capping layer and the second capping layer may each independently be an organic capping layer including an organic material, an inorganic capping layer including an inorganic material, or an organic-inorganic composite capping layer including an organic material and an inorganic material.

At least one of the first capping layer or the second capping layer may each independently include carbocyclic compounds, heterocyclic compounds, amine group-containing compounds, porphyrin derivatives, phthalocyanine derivatives, naphthalocyanine derivatives, alkali metal complexes, alkaline earth metal complexes, or any suitable combinations thereof. The carbocyclic compound, the heterocyclic compound, and the amine group-containing compound may be optionally substituted with a substituent containing O, N, S, Se, Si, F, Cl, Br, I, or any suitable combination thereof. In an embodiment, at least one of the first capping layer or the second capping layer may each independently include an amine group-containing compound.

In an embodiment, at least one of the first capping layer and the second capping layer may each independently include a compound represented by Formula 201, a compound represented by Formula 202, or any suitable combinations thereof.

In an embodiment, at least one of the first capping layer and the second capping layer may each independently include one of Compounds HT28 to HT33, one of Compounds CP1 to CP6, β-NPB, or any suitable combinations thereof:

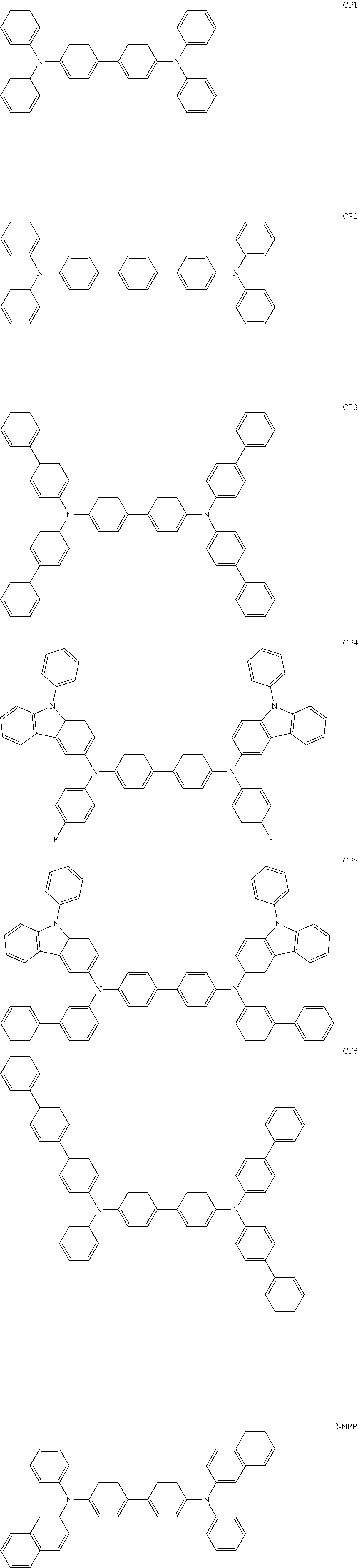

### Film

The diamine-based compound represented by Formula 1 may be included in various suitable films. Therefore, according to one or more embodiments, a film including the diamine-based compound represented by Formula 1 may be provided. The film may be, for example, an optical member (or, a light-controlling member) (e.g., a color filter, a color-conversion member, a capping layer, a light extraction efficiency improvement layer, a selective light-absorbing layer, a polarizing layer, a quantum dot-containing layer, or the like), a light-blocking member (e.g., a light reflection layer or a light-absorbing layer), or a protection member (e.g., an insulating layer or a dielectric material layer).

### Electronic Apparatus

The light-emitting device may be included in various electronic apparatuses. In an embodiment, the electronic apparatus including the light-emitting device may be a light-emitting apparatus, an authentication apparatus, or the like.

The electronic apparatus (for example, light-emitting apparatus) may further include, in addition to the light-emitting device, i) a color filter, ii) a color conversion layer, or iii) a color filter and a color conversion layer. The color filter and/or the color conversion layer may be located in at least one traveling direction of light emitted from the light-emitting device. For example, the light emitted from the light-emitting device may be blue light or white light. The light-emitting device may be the same as described above. In an embodiment, the color conversion layer may include quantum dots. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be located among the subpixel areas to define each of the subpixel areas.

The color filter may further include a plurality of color filter areas and light-shielding patterns located among the color filter areas, and the color conversion layer may include a plurality of color conversion areas and light-shielding patterns located among the color conversion areas.

The color filter areas (or the color conversion areas) may include a first area emitting first color light, a second area emitting second color light, and/or a third area emitting third color light, and the first color light, the second color light, and/or the third color light may have different maximum emission wavelengths from one another. In an embodiment, the first color light may be red light, the second color light may be green light, and the third color light may be blue light. In an embodiment, the color filter areas (or the color conversion areas) may include quantum dots. In more detail, the first area may include a red quantum dot, the second area may include a green quantum dot, and the third area may not include a quantum dot. The quantum dot is the same as described in the present specification. The first area, the second area, and/or the third area may each further include a scatterer.

In an embodiment, the light-emitting device may emit first light, the first area may absorb the first light to emit first first-color light, the second area may absorb the first light to emit second first-color light, and the third area may absorb the first light to emit third first-color light. In this regard, the first first-color light, the second first-color light, and the third first-color light may have different maximum emission wavelengths. In detail, the first light may be blue light, the first first-color light may be red light, the second first-color light may be green light, and the third first-color light may be blue light.

The electronic apparatus may further include a thin-film transistor in addition to the light-emitting device as described above. The thin-film transistor may include a source electrode, a drain electrode, and an activation layer, wherein any one of the source electrode and the drain electrode may be electrically connected to any one of the first electrode and the second electrode of the light-emitting device.

The thin-film transistor may further include a gate electrode, a gate insulating film, etc.

The activation layer may include crystalline silicon, amorphous silicon, organic semiconductor, oxide semiconductor, and/or the like.

The electronic apparatus may further include a sealing portion for sealing the light-emitting device. The sealing portion and/or the color conversion layer may be located between the color filter and the light-emitting device. The sealing portion allows light from the light-emitting device to be extracted to the outside, and concurrently (e.g., simultaneously) prevents (reduces) ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic apparatus may be flexible.

Various suitable functional layers may be additionally located on the sealing portion, in addition to the color filter and/or the color conversion layer, according to the intended use of the electronic apparatus. The functional layers may include a touch screen layer, a polarizing layer, and the like. The touch screen layer may be a pressure-sensitive touch screen layer, a capacitive touch screen layer, or an infrared touch screen layer. The authentication apparatus may be, for example, a biometric authentication apparatus that authenticates an individual by using biometric information of a living body (for example, fingertips, pupils, etc.).

The authentication apparatus may further include, in addition to the light-emitting device, a biometric information collector.

The electronic apparatus may be applied to various suitable displays, light sources, lighting, personal computers (for example, a mobile personal computer), mobile phones, digital cameras, electronic diaries, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, various measuring instruments, meters (for example, meters for a vehicle, an aircraft, and a vessel), projectors, and the like.

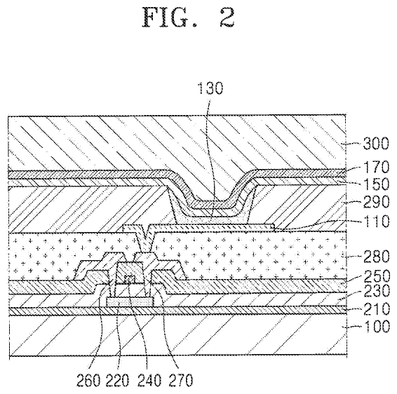

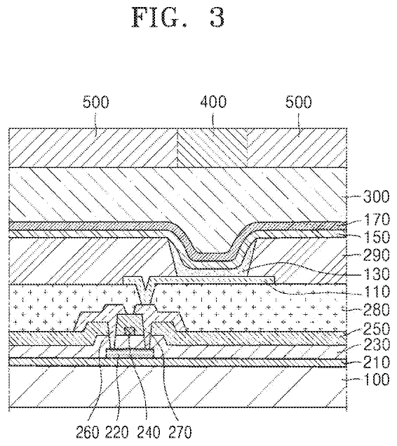

### Description of FIGS. 2 and 3

FIG. 2 is a cross-sectional view of a light-emitting apparatus according to an embodiment of the disclosure.

The light-emitting apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be formed on the substrate 100. The buffer layer 210 may prevent (reduce) penetration of impurities through the substrate 100 and may provide a flat surface on the substrate 100.

A TFT may be located on the buffer layer 210. The TFT may include an activation layer 220, a gate electrode 240, a source electrode 260, and a drain electrode 270.

The activation layer 220 may include an inorganic semiconductor such as silicon or polysilicon, an organic semiconductor, or an oxide semiconductor, and may include a source region, a drain region and a channel region.

A gate insulating film 230 for insulating the activation layer 220 from the gate electrode 240 may be located on the activation layer 220, and the gate electrode 240 may be located on the gate insulating film 230.

An interlayer insulating film 250 is located on the gate electrode 240. The interlayer insulating film 250 may be placed between the gate electrode 240 and the source electrode 260 to insulate the gate electrode 240 from the source electrode 260 and between the gate electrode 240 and the drain electrode 270 to insulate the gate electrode 240 from the drain electrode 270.

The source electrode 260 and the drain electrode 270 may be located on the interlayer insulating film 250. The interlayer insulating film 250 and the gate insulating film 230 may be formed to expose the source region and the drain region of the activation layer 220, and the source electrode 260 and the drain electrode 270 may be in contact with the exposed portions of the source region and the drain region of the activation layer 220.

The TFT is electrically connected to a light-emitting device to drive the light-emitting device, and is covered by a passivation layer 280. The passivation layer 280 may include an inorganic insulating film, an organic insulating film, or a combination thereof. A light-emitting device is provided on the passivation layer 280. The light-emitting device may include a first electrode 110, an interlayer 130, and a second electrode 150.

The first electrode 110 may be formed on the passivation layer 280. The passivation layer 280 does not completely cover the drain electrode 270 and exposes a portion of the drain electrode 270, and the first electrode 110 is connected to the exposed portion of the drain electrode 270.

A pixel-defining layer 290 containing an insulating material may be located on the first electrode 110. The pixel-defining layer 290 exposes a region of the first electrode 110, and an interlayer 130 may be formed in the exposed region of the first electrode 110. The pixel-defining layer 290 may be a polyimide or polyacrylic organic film. At least some layers of the interlayer 130 may extend beyond the upper portion of the pixel-defining layer 290 to be located in the form of a common layer.

The second electrode 150 may be located on the interlayer 130, and a capping layer 170 may be additionally formed on the second electrode 150. The capping layer 170 may be formed to cover the second electrode 150.

The encapsulation portion 300 may be located on the capping layer 170. The encapsulation portion 300 may be located on a light-emitting device to protect the light-emitting device from moisture or oxygen. The encapsulation portion 300 may include: an inorganic film including silicon nitride (SiNx), silicon oxide (SiOx), indium tin oxide, indium zinc oxide, or any suitable combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, or the like), an epoxy-based resin (for example, aliphatic glycidyl ether (AGE), or the like), or combinations thereof; or a combination of the inorganic film and the organic film.

FIG. 3 is a cross-sectional view of a light-emitting apparatus according to an embodiment of the disclosure.

The light-emitting apparatus of FIG. 3 is the same as the light-emitting apparatus of FIG. 2, except that a light-shielding pattern 500 and a functional region 400 are additionally located on the encapsulation portion 300. The functional region 400 may be a combination of i) a color filter area, ii) a color conversion area, or iii) a combination of the color filter area and the color conversion area. In an embodiment, the light-emitting device included in the light-emitting apparatus of FIG. 3 may be a tandem light-emitting device.

### Manufacture Method

Respective layers included in the hole transport region, the emission layer, and respective layers included in the electron transport region may be formed in a certain region by using one or more suitable methods selected from vacuum deposition, spin coating, casting, Langmuir-Blodgett (LB) deposition, ink-jet printing, laser-printing, and laser-induced thermal imaging and other methods that should be apparent to one of ordinary skill in the art upon reviewing the disclosure.

When layers constituting the hole transport region, the emission layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group” as used herein refers to a cyclic group consisting of carbon only as a ring-forming atom and having three to sixty carbon atoms, and the term “C₁-C₆₀ heterocyclic group” as used herein refers to a cyclic group that has one to sixty carbon atoms and further has, in addition to carbon, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. In an embodiment, the C₁-C₆₀ heterocyclic group has 3 to 61 ring-forming atoms.

The “cyclic group” as used herein may include the C₃-C₆₀ carbocyclic group and the C₁-C₆₀ heterocyclic group.

The term “π electron-rich C₃-C₆₀ cyclic group” as used herein refers to a cyclic group that has three to sixty carbon atoms and does not include *—N═*′ as a ring-forming moiety, and the term “π electron-deficient nitrogen-containing C₁-C₆₀ cyclic group” as used herein refers to a heterocyclic group that has one to sixty carbon atoms and includes *—N═*′ as a ring-forming moiety.

In an embodiment,

the C₃-C₆₀ carbocyclic group may be i) group T1 or ii) a condensed cyclic group in which two or more groups T1 are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) group T2, ii) a condensed cyclic group in which two or more groups T2 are condensed with each other, or iii) a condensed cyclic group in which at least one group T2 and at least one group T1 are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

the π electron-rich C₃-C₆₀ cyclic group may be i) group T1, ii) a condensed cyclic group in which two or more groups T1 are condensed with each other, iii) group T3, iv) a condensed cyclic group in which two or more groups T3 are condensed with each other, or v) a condensed cyclic group in which at least one group T3 and at least one group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),

the π electron-deficient nitrogen-containing C₁-C₆₀ cyclic group may be i) group T4, ii) a condensed cyclic group in which two or more group T4 are condensed with each other, iii) a condensed cyclic group in which at least one group T4 and at least one group T1 are condensed with each other, iv) a condensed cyclic group in which at least one group T4 and at least one group T3 are condensed with each other, or v) a condensed cyclic group in which at least one group T4, at least one group T1, and at least one group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,

group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,

group T3 may be a furan group, a thiophene group, a 1H-pyrrole group, a silole group, or a borole group, and

group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The term “cyclic group”, “C₃-C₆₀ carbocyclic group”, “C₁-C₆₀ heterocyclic group”, “π electron-rich C₃-C₆₀ cyclic group”, or “π electron-deficient nitrogen-containing C₁-C₆₀ cyclic group” as used herein refers to a group condensed to any cyclic group or a polyvalent group (for example, a divalent group, a trivalent group, a tetravalent group, etc.), depending on the structure of a formula in connection with which the terms are used. In an embodiment, “a benzene group” may be a benzo group, a phenyl group, a phenylene group, or the like, which may be easily understood by one of ordinary skill in the art according to the structure of a formula including the “benzene group.”

Examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group, and examples of the divalent C₃-C₆₀ carbocyclic group and the monovalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as used herein refers to a linear or branched aliphatic hydrocarbon monovalent group that has one to sixty carbon atoms, and examples thereof include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as used herein refers to a divalent group having the same structure as the C₁-C₆₀ alkyl group.

The term “C₂-C₆₀ alkenyl group” as used herein refers to a monovalent hydrocarbon group having at least one carbon-carbon double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of the C₂-C₆₀ alkyl group, and examples thereof include an ethynyl group and a propynyl group. The term “C₂-C₆₀ alkynylene group” as used herein refers to a divalent group having the same structure as the C₂-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group” as used herein refers to a monovalent saturated hydrocarbon cyclic group having 3 to 10 carbon atoms, and examples thereof include a cyclopropyl group, a cyclobutyl group, a cyclopentyl group, a cyclohexyl group, a cycloheptyl group, a cyclooctyl group, an adamantanyl group, a norbornanyl group (or a bicyclo[2.2.1]heptyl group), a bicyclo[1.1.1]pentyl group, a bicyclo[2.1.1]hexyl group, and a bicyclo[2.2.2]octyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent cyclic group that further includes, in addition to a carbon atom, at least one heteroatom as a ring-forming atom and has 1 to 10 carbon atoms, and examples thereof include a 1,2,3,4-oxatriazolidinyl group, a tetrahydrofuranyl group, and a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” used herein refers to a monovalent cyclic group that has three to ten carbon atoms and at least one carbon-carbon double bond in the ring thereof and no aromaticity, and examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent cyclic group that has, in addition to a carbon atom, at least one heteroatom as a ring-forming atom, 1 to 10 carbon atoms, and at least one carbon-carbon double bond in the cyclic structure thereof. Examples of the C₁-C₁₀ heterocycloalkenyl group include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system having six to sixty carbon atoms, and the term “C₆-C₆₀ arylene group” as used herein refers to a divalent group having a carbocyclic aromatic system having six to sixty carbon atoms. Examples of the C₆-C₆₀ aryl group include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the rings may be condensed with each other.

The term “C₁-C₆₀ heteroaryl group” as used herein refers to a monovalent group having a heterocyclic aromatic system that has, in addition to a carbon atom, at least one heteroatom as a ring-forming atom, and 1 to 60 carbon atoms. The term “C₁-C₆₀ heteroarylene group” as used herein refers to a divalent group having a heterocyclic aromatic system that has, in addition to a carbon atom, at least one heteroatom as a ring-forming atom, and 1 to 60 carbon atoms. Examples of the C₁-C₆₀ heteroaryl group include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the rings may be condensed with each other.

The term “monovalent non-aromatic condensed polycyclic group” as used herein refers to a monovalent group having two or more rings condensed to each other, only carbon atoms (for example, having 8 to 60 carbon atoms) as ring-forming atoms, and non-aromaticity in its molecular structure when considered as a whole. Examples of the monovalent non-aromatic condensed polycyclic group include an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as used herein refers to a divalent group having the same structure as a monovalent non-aromatic condensed polycyclic group.

The term “monovalent non-aromatic condensed heteropolycyclic group” as used herein refers to a monovalent group having two or more rings condensed to each other, at least one heteroatom other than carbon atoms (for example, having 1 to 60 carbon atoms), as a ring-forming atom, and non-aromaticity in its molecular structure when considered as a whole. Examples of the monovalent non-aromatic condensed heteropolycyclic group include a pyrrolyl group, a thiophenyl group, a furanyl group, an indolyl group, a benzoindolyl group, a naphtho indolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group” as used herein refers to a divalent group having the same structure as a monovalent non-aromatic condensed heteropolycyclic group.

The term “C₆-C₆₀ aryloxy group” as used herein indicates —OA₁₀₂ (wherein A₁₀₂ is the C₆-C₆₀ aryl group), and the term “C₆-C₆₀ arylthio group” as used herein indicates —SA₁₀₃ (wherein A₁₀₃ is the C₆-C₆₀ aryl group).

The term “C₇-C₆₀ aryl alkyl group” used herein refers to -A₁₀₄A₁₀₅ (where A104 may be a C₁-C₅₄ alkylene group, and A10s may be a C₆-C₅₉ aryl group), and the term “C₂-C₆₀ heteroaryl alkyl group” used herein refers to -A₁₀₆A₁₀₇ (where A₁₀₆ may be a C₁-C₅₉ alkylene group, and A₁₀₇ may be a C₁-C₅₉ heteroaryl group).

R₁₀ₐ may be:

deuterium (-D), —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇—Co aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any suitable combination thereof;

a C₃-C₆₀ carbocyclic group, a C₁-C₆₀ heterocyclic group, a C₆-C₆₀ aryloxy group, a C₆-C₆₀ arylthio group, a C₇—Co aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇—Co aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any suitable combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃, and Q₃₁ to Q₃₃ used herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any suitable combination thereof; a C₇-C₆₀ aryl alkyl group; or a C₂-C₆₀ heteroaryl alkyl group.

The term “hetero atom” as used herein refers to any atom other than a carbon atom. Examples of the heteroatom include O, S, N, P, Si, B, Ge, Se, or any suitable combination thereof.

The term “the third-row transition metal” used herein includes hafnium (Hf), tantalum (Ta), tungsten (W), rhenium (Re), osmium (Os), iridium (Ir), platinum (Pt), gold (Au), and the like.

“Ph” as used herein refers to a phenyl group, “Me” as used herein refers to a methyl group, “Et” as used herein refers to an ethyl group, “tert-Bu” or “Bu^{t}” as used herein refers to a tert-butyl group, and “OMe” as used herein refers to a methoxy group.

The term “biphenyl group” as used herein refers to “a phenyl group substituted with a phenyl group.” In other words, the “biphenyl group” is a substituted phenyl group having a C₆-C₆₀ aryl group as a substituent.

The term “terphenyl group” as used herein refers to “a phenyl group substituted with a biphenyl group”. The “terphenyl group” is a substituted phenyl group having, as a substituent, a C₆-C₆₀ aryl group substituted with a C₆-C₆₀ aryl group.

* and *′ as used herein, unless defined otherwise, each refer to a binding site to a neighboring atom in a corresponding formula or moiety.

Hereinafter, compounds according to embodiments and light-emitting devices according to embodiments will be described in more detail with reference to the following synthesis examples and examples. The wording “B was used instead of A” used in describing Synthesis Examples means that an identical molar equivalent of B was used in place of A.

### EXAMPLE

### Synthesis Example 1

### Synthesis Example 1: Synthesis of Compound 1

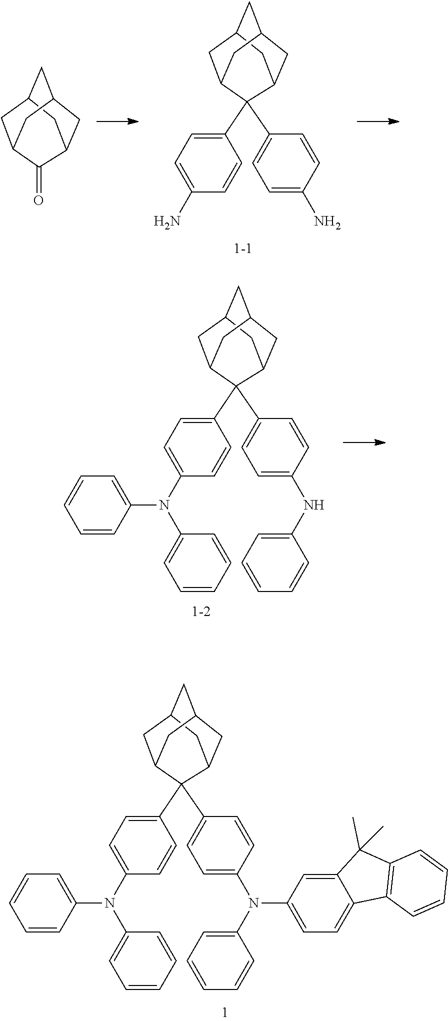 [SMILES: O=C1C2CC3CC(C2)CC1C3 ; Nc1ccc(C2(c3ccc(N)cc3)C3CC4CC(C3)CC2C4)cc1 ; c1ccc(Nc2ccc(C3(c4ccc(N(c5ccccc5)c5ccccc5)cc4)C4CC5CC(C4)CC3C5)cc2)cc1 ; CC1(C)c2ccccc2-c2ccc(N(c3ccccc3)c3ccc(C4(c5ccc(N(c6ccccc6)c6ccccc6)cc5)C5CC6CC(C5)CC4C6)cc3)cc21]

### Synthesis of Intermediate 1-1

1.50 g (10.0 mmol) of adamantan-2-one and 3.89 g (30 mmol) of anilinium chloride were dissolved in 5 ml of aniline, and then stirred for 72 hours at 190° C. The reaction solution was cooled at room temperature, followed by three times of an extraction process using 60 ml of water and DCM (30 ml). The collected DCM was dried using MgSO₄ and then dried under reduced pressure to obtain a product, which was then separated and purified by silica gel column chromatography, to thereby obtain 0.95 g (yield: 30%) of Intermediate 1-1 as a white solid. The resulting compound was identified by LC-MS. C₁₀H₁₄O: M+150.2

### Synthesis of Intermediate 1-2

3.50 g (11.0 mmol) of Intermediate 1-1, 3.34 ml (30 mmol) of iodobenzene, 1.37 g (1.5 mmol) of tris(dibenzylideneacetone)dipalladium(0) (Pd₂dba₃), 0.60 g (3 mmol) of P(t-Bu)₃, and 8.64 g (90 mmol) of sodium tert-butoxide were dissolved in 120 ml of toluene and then stirred for 3 hours at 80° C. The reaction solution was cooled at room temperature, and then, 40 ml of water added thereto, followed by three times of an extraction process using 50 ml of ethyl ether. The collected ethyl ether was dried using MgSO₄, the residue obtained by evaporating the solvent was separated and purified by silica gel column chromatography, to thereby obtain 2.74 g (yield: 50%) of Intermediate 1-2. The resulting compound was identified by LC-MS. C₄₀H₃₈N₂: M+576.7

### Synthesis of Compound 1

5.47 g (10.0 mmol) of Intermediate 1-2, 2.73 g (10 mmol) of 2-bromo-9,9-dimethyl-9H-fluorene, 0.46 g (0.5 mmol) of tris(dibenzylideneacetone)dipalladium(0) (Pd₂dba₃), 0.24 g (1 mmol) of P(t-Bu)₃, and 2.88 g (30 mmol) of sodium tert-butoxide were dissolved in 60 ml of toluene, and then stirred for 3 hours at 80° C. The reaction solution was cooled at room temperature, and then, 40 ml of water added thereto, followed by three times of an extraction process using 50 ml of ethyl ether. The collected ethyl ether was dried using MgSO₄, the residue obtained by evaporating the solvent was separated and purified by silica gel column chromatography, to thereby obtain 54 g (yield: 65%) of Compound 1. The resulting compound was identified by MS/FAB and 1H NMR.

### Synthesis Example 2: Synthesis of Compound 13

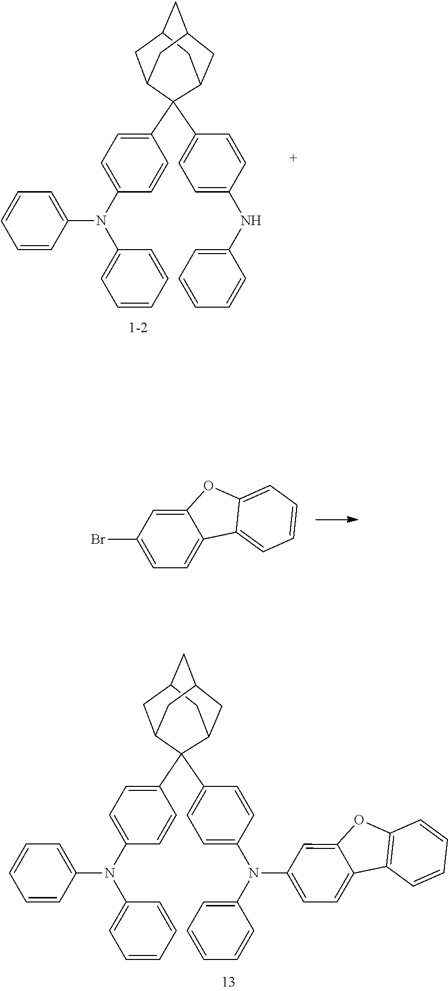 [SMILES: c1ccc(Nc2ccc(C3(c4ccc(N(c5ccccc5)c5ccccc5)cc4)C4CC5CC(C4)CC3C5)cc2)cc1 ; Brc1ccc2c(c1)oc1ccccc12 ; c1ccc(N(c2ccccc2)c2ccc(C3(c4ccc(N(c5ccccc5)c5ccc6c(c5)oc5ccccc56)cc4)C4CC5CC(C4)CC3C5)cc2)cc1]

Compound 13 was synthesized in the same manner as in Synthesis Example of Compound 1, except that 3-bromodibenzo[b,d]furan was used instead of 2-bromo-9,9-dimethyl-9H-fluorene. The resulting compound was identified by MS/FAB and 1H NMR.

### Synthesis Example 3: Synthesis of Compound 19

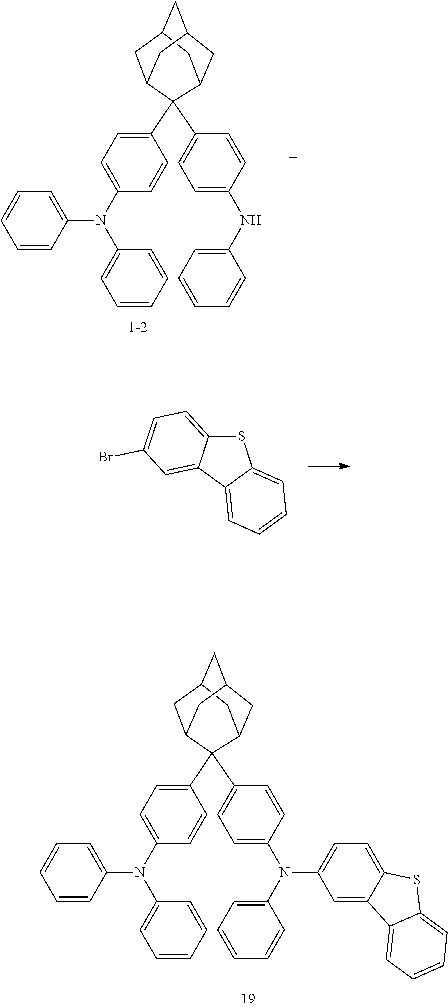 [SMILES: c1ccc(Nc2ccc(C3(c4ccc(N(c5ccccc5)c5ccccc5)cc4)C4CC5CC(C4)CC3C5)cc2)cc1 ; Brc1ccc2sc3ccccc3c2c1 ; c1ccc(N(c2ccccc2)c2ccc(C3(c4ccc(N(c5ccccc5)c5ccc6sc7ccccc7c6c5)cc4)C4CC5CC(C4)CC3C5)cc2)cc1]

Compound 19 was synthesized in the same manner as in Synthesis Example of Compound 1, except that 2-bromodibenzo[b,d]thiophene was used instead of 2-bromo-9,9-dimethyl-9H-fluorene. The resulting compound was identified by MS/FAB and 1H NMR.

### Synthesis Example 4: Synthesis of Compound 21

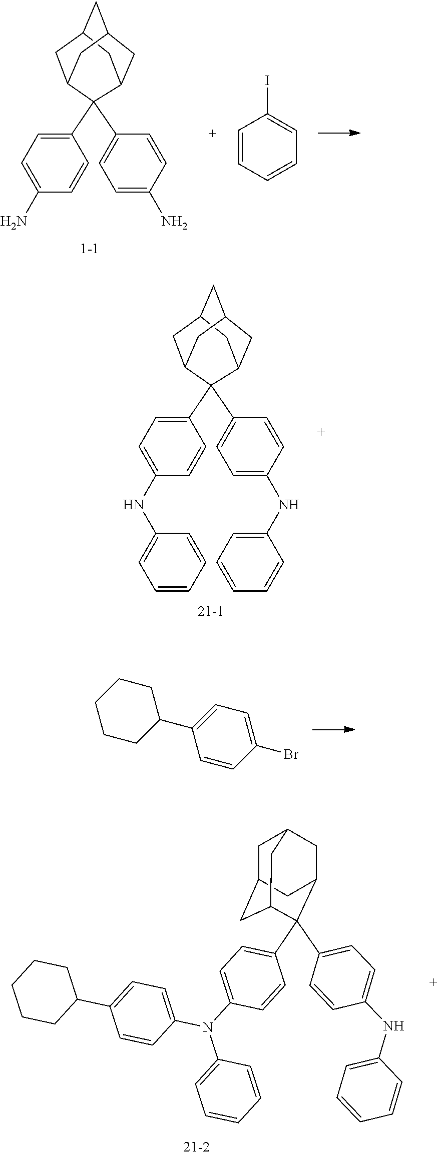 [SMILES: Nc1ccc(C2(c3ccc(N)cc3)C3CC4CC(C3)CC2C4)cc1 ; Ic1ccccc1 ; c1ccc(Nc2ccc(C3(c4ccc(Nc5ccccc5)cc4)C4CC5CC(C4)CC3C5)cc2)cc1 ; Brc1ccc(C2CCCCC2)cc1 ; c1ccc(Nc2ccc(C3(c4ccc(N(c5ccccc5)c5ccc(C6CCCCC6)cc5)cc4)C4CC5CC(C4)CC3C5)cc2)cc1]

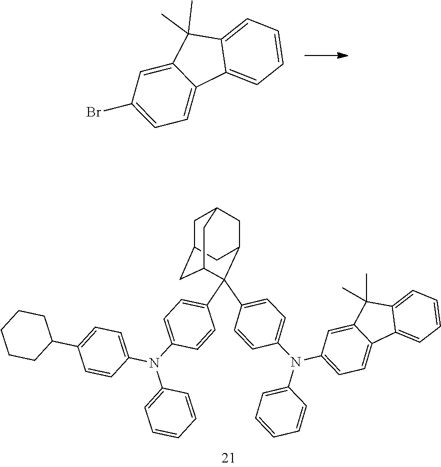 [SMILES: CC1(C)c2ccccc2-c2ccc(Br)cc21 ; CC1(C)c2ccccc2-c2ccc(N(c3ccccc3)c3ccc(C4(c5ccc(N(c6ccccc6)c6ccc(C7CCCCC7)cc6)cc5)C5CC6CC(C5)CC4C6)cc3)cc21]

### Synthesis of Intermediate 21-1

3.50 g (11.0 mmol) of Intermediate 1-1, 2.23 ml (20 mmol) of iodobenzene, 0.46 g (0.5 mmol) of tris(dibenzylideneacetone)dipalladium(0) (Pd₂dba₃), 0.24 g (1 mmol) of P(t-Bu)₃, and 2.88 g (30 mmol) of sodium tert-butoxide were dissolved in 60 ml of toluene and then stirred for 3 hours at 80° C. The reaction solution was cooled at room temperature, and then, 40 ml of water added thereto, followed by three times of an extraction process using 50 ml of ethyl ether. The collected ethyl ether was dried using MgSO₄, the residue obtained by evaporating the solvent was separated and purified by silica gel column chromatography, to thereby obtain 3.30 g (yield: 70%) of Intermediate 21-1. The resulting compound was identified by LC-MS. C₃₄H₃₄N₂: M+470.

### Synthesis of Intermediate 21-2

5.18 g (11.0 mmol) of Intermediate 21-1, 2.39 g (10 mmol) of 1-bromo-4-cyclohexylbenzene, 0.46 g (0.5 mmol) of tris(dibenzylideneacetone)dipalladium(0) (Pd₂dba₃), 0.24 g (1 mmol) of P(t-Bu)₃, and 2.88 g (30 mmol) of sodium tert-butoxide were dissolved in 60 ml of toluene, and then stirred for 3 hours at 80° C. The reaction solution was cooled at room temperature, and then, 40 ml of water added thereto, followed by three times of an extraction process using 50 ml of ethyl ether. The collected ethyl ether was dried using MgSO₄, the residue obtained by evaporating the solvent was separated and purified by silica gel column chromatography, to thereby obtain 3.46 g (yield: 55%) of Intermediate 21-2. The resulting compound was identified by LC-MS. C₄₆H₄₈N₂: M+628.9

### Synthesis of Compound 21

6.29 g (10.0 mmol) of Intermediate 21-2, 2.73 g (10 mmol) of 2-bromo-9,9-dimethyl-9H-fluorene, 0.46 g (0.5 mmol) of tris(dibenzylideneacetone)dipalladium(0) (Pd₂dba₃), 0.24 g (1 mmol) of P(t-Bu)₃, and 2.88 g (30 mmol) of sodium tert-butoxide were dissolved in 60 ml of toluene, and then stirred for 3 hours at 80° C. The reaction solution was cooled at room temperature, and then, 40 ml of water added thereto, followed by three times of an extraction process using 50 ml of ethyl ether. The collected ethyl ether was dried using MgSO₄, the residue obtained by evaporating the solvent was separated and purified by silica gel column chromatography, to thereby obtain 5.74 g (yield: 70%) of Compound 21. The resulting compound was identified by MS/FAB and 1H NMR.

### Synthesis Example 5: Synthesis of Compound 28

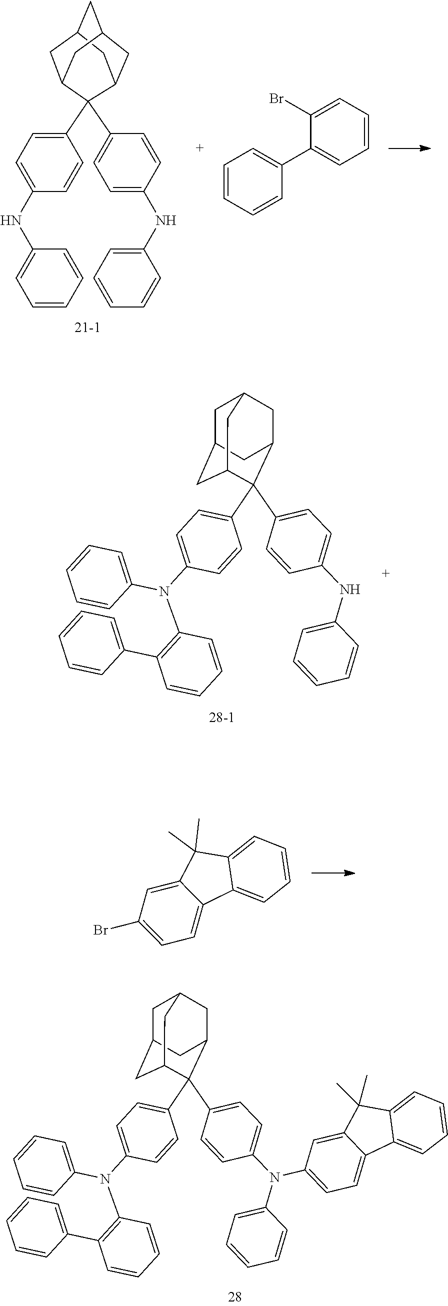 [SMILES: c1ccc(Nc2ccc(C3(c4ccc(Nc5ccccc5)cc4)C4CC5CC(C4)CC3C5)cc2)cc1 ; Brc1ccccc1-c1ccccc1 ; c1ccc(Nc2ccc(C3(c4ccc(N(c5ccccc5)c5ccccc5-c5ccccc5)cc4)C4CC5CC(C4)CC3C5)cc2)cc1 ; CC1(C)c2ccccc2-c2ccc(Br)cc21 ; CC1(C)c2ccccc2-c2ccc(N(c3ccccc3)c3ccc(C4(c5ccc(N(c6ccccc6)c6ccccc6-c6ccccc6)cc5)C5CC6CC(C5)CC4C6)cc3)cc21]

### Synthesis of Intermediate 28-1

5.18 g (11.0 mmol) of Intermediate 21-1, 2.33 g (10 mmol) of 2-bromo-1,1′-biphenyl, 0.46 g (0.5 mmol) of tris(dibenzylideneacetone)dipalladium(0) (Pd₂dba₃), 0.24 g (1 mmol) of P(t-Bu)₃, and 2.88 g (30 mmol) of sodium tert-butoxide were dissolved in 60 ml of toluene, and then stirred for 3 hours at 80° C. The reaction solution was cooled at room temperature, and then, 40 ml of water added thereto, followed by three times of an extraction process using 50 ml of ethyl ether. The collected ethyl ether was dried using MgSO₄, the residue obtained by evaporating the solvent was separated and purified by silica gel column chromatography, to thereby obtain 3.86 g (yield: 62%) of Intermediate 28-1. The resulting compound was identified by LC-MS. C₄₆H₄₈N₂: M+622.8

### Synthesis of Compound 28

6.23 g (10.0 mmol) of Intermediate 28-1, 2.73 g (10 mmol) of 2-bromo-9,9-dimethyl-9H-fluorene, 0.46 g (0.5 mmol) of tris(dibenzylideneacetone)dipalladium(0) (Pd₂dba₃), 0.24 g (1 mmol) of P(t-Bu)₃, and 2.88 g (30 mmol) of sodium tert-butoxide were dissolved in 60 ml of toluene, and then stirred for 3 hours at 80° C. The reaction solution was cooled at room temperature, and then, 40 ml of water added thereto, followed by three times of an extraction process using 50 ml of ethyl ether. The collected ethyl ether was dried using MgSO₄, the residue obtained by evaporating the solvent was separated and purified by silica gel column chromatography, to thereby obtain 5.71 g (yield: 70%) of Compound 28. The resulting compound was identified by MS/FAB and 1H NMR.

### Synthesis Example 6: Synthesis of Compound 30

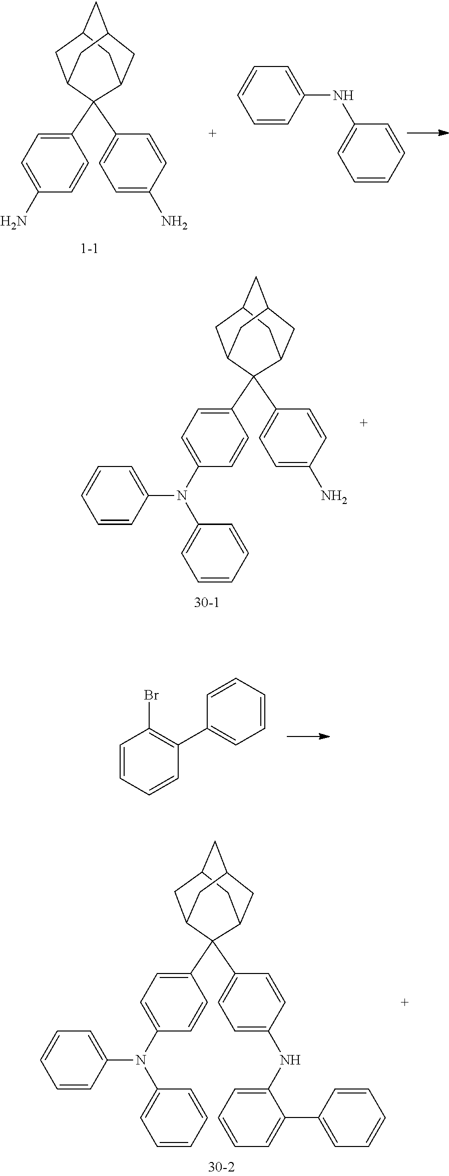 [SMILES: Nc1ccc(C2(c3ccc(N)cc3)C3CC4CC(C3)CC2C4)cc1 ; c1ccc(Nc2ccccc2)cc1 ; Nc1ccc(C2(c3ccc(N(c4ccccc4)c4ccccc4)cc3)C3CC4CC(C3)CC2C4)cc1 ; Brc1ccccc1-c1ccccc1 ; c1ccc(-c2ccccc2Nc2ccc(C3(c4ccc(N(c5ccccc5)c5ccccc5)cc4)C4CC5CC(C4)CC3C5)cc2)cc1]

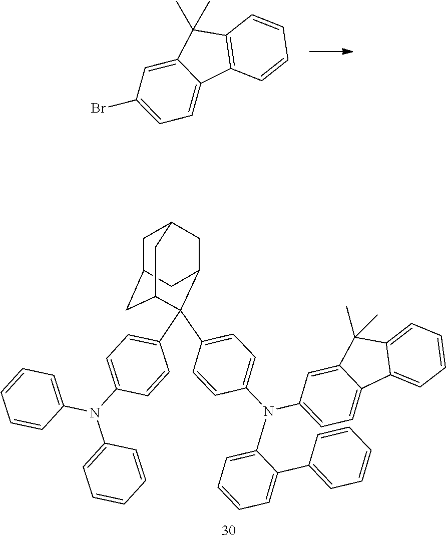

### Synthesis of Intermediate 30-1

3.50 g (11.0 mmol) of Intermediate 1-1, 1.69 g (10 mmol) of diphenylamine, 0.46 g (0.5 mmol) of tris(dibenzylideneacetone)dipalladium(0) (Pd₂dba₃), 0.55 g (1 mmol) of 1,1′-Bis (diphenylphosphino) ferrocene, and 2.88 g (30 mmol) of sodium tert-butoxide were dissolved in 60 ml of toluene, and then stirred for 3 hours at 80° C. The reaction solution was cooled at room temperature, and then, 40 ml of water added thereto, followed by three times of an extraction process using 50 ml of ethyl ether. The collected ethyl ether was dried using MgSO₄, the residue obtained by evaporating the solvent was separated and purified by silica gel column chromatography, to thereby obtain 2.35 g (yield: 50%) of Intermediate 30-1. The resulting compound was identified by LC-MS. C₃₄H₃₄N₂: M+470.6

### Synthesis of Intermediate 30-2

5.18 g (11.0 mmol) of Intermediate 30-1, 2.33 g (10 mmol) of 2-bromo-1,1′-biphenyl, 0.46 g (0.5 mmol) of tris(dibenzylideneacetone)dipalladium(0) (Pd₂dba₃), 0.24 g (1 mmol) of P(t-Bu)₃, and 2.88 g (30 mmol) of sodium tert-butoxide were dissolved in 60 ml of toluene, and then stirred for 3 hours at 80° C. The reaction solution was cooled at room temperature, and then, 40 ml of water added thereto, followed by three times of an extraction process using 50 ml of ethyl ether. The collected ethyl ether was dried using MgSO₄, the residue obtained by evaporating the solvent was separated and purified by silica gel column chromatography, to thereby obtain 4.67 g (yield: 75%) of Intermediate 30-2. The resulting compound was identified by LC-MS. C₄₈H₄₂N₂: M+622.8

### Synthesis of Compound 30

6.23 g (10.0 mmol) of Intermediate 30-2, 2.73 g (10 mmol) of 2-bromo-9,9-dimethyl-9H-fluorene, 0.46 g (0.5 mmol) of tris(dibenzylideneacetone)dipalladium(0) (Pd₂dba₃), 0.24 g (1 mmol) of P(t-Bu)₃, and 2.88 g (30 mmol) of sodium tert-butoxide were dissolved in 60 ml of toluene, and then stirred for 3 hours at 80° C. The reaction solution was cooled at room temperature, and then, 40 ml of water added thereto, followed by three times of an extraction process using 50 ml of ethyl ether. The collected ethyl ether was dried using MgSO₄, the residue obtained by evaporating the solvent was separated and purified by silica gel column chromatography, to thereby obtain 5.71 g (yield: 70%) of Compound 30. The resulting compound was identified by MS/FAB and 1H NMR.

### Synthesis Example 7: Synthesis of Compound 38

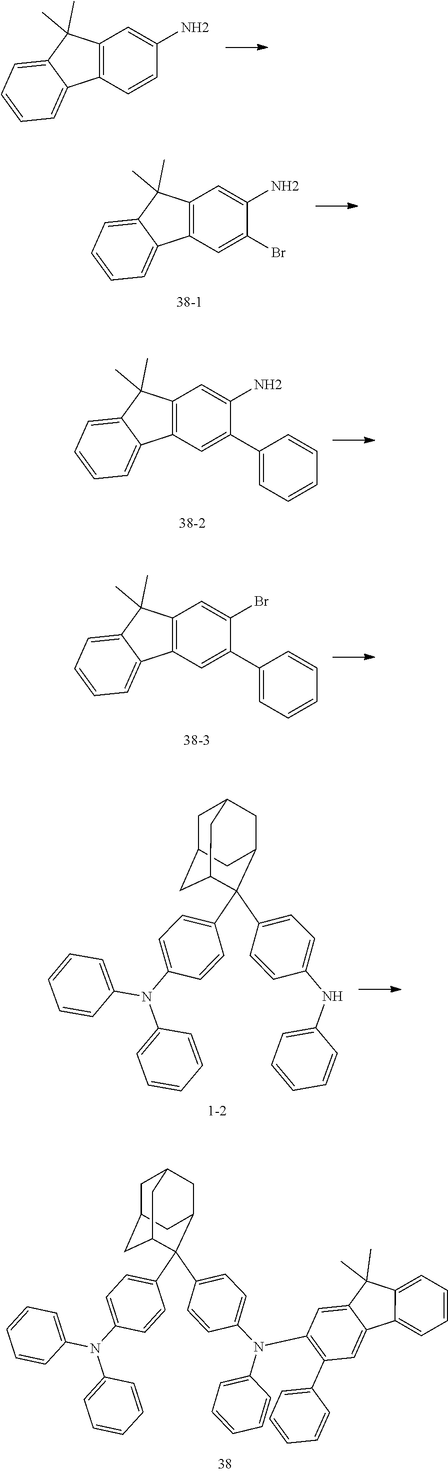 [SMILES: CC1(C)c2ccccc2-c2ccc(N)cc21 ; CC1(C)c2ccccc2-c2cc(Br)c(N)cc21 ; CC1(C)c2ccccc2-c2cc(-c3ccccc3)c(N)cc21 ; CC1(C)c2ccccc2-c2cc(-c3ccccc3)c(Br)cc21 ; c1ccc(Nc2ccc(C3(c4ccc(N(c5ccccc5)c5ccccc5)cc4)C4CC5CC(C4)CC3C5)cc2)cc1 ; CC1(C)c2ccccc2-c2cc(-c3ccccc3)c(N(c3ccccc3)c3ccc(C4(c5ccc(N(c6ccccc6)c6ccccc6)cc5)C5CC6CC(C5)CC4C6)cc3)cc21]

### Synthesis of Intermediate 38-1

2.09 g (10 mmol) of 9,9-dimethyl-9H-fluoren-2-amine was dissolved in 20 ml of DCM, and N-bromosuccinimide (1.78 g, in DCM) was added thereto at 0° C. The resultant was stirred for 5 hours at room temperature, and then, 3 g of Na₂S₂O₃ was dissolved in water and added thereto, followed by three times of washing using DCM (30 ml). The washed DCM layer was dried using MgSO₄ and then dried under reduced pressure to obtain a product, which was then separated and purified by silica gel column chromatography, to thereby obtain 2.30 g (yield: 80%) of Intermediate 38-1 as a white solid. The resulting compound was identified by LC-MS. C₁₅H₁₄BrN: M⁺288.1

### Synthesis of Intermediate 38-2

2.88 g (10.0 mmol) of Intermediate 38-1, 1.46 g (12.0 mmol) of phenylboronic acid, 0.58 g (0.5 mmol) of Pd(PPh₃)₄, and 4.14 g (30.0 mmol) of K₂CO₃ were dissolved in 60 ml of a THF/H₂O (2/1) mixed solution, and then stirred for 16 hours at 80° C. The reaction solution was cooled at room temperature, followed by three times of an extraction process using 60 ml of water and 60 ml of diethyl ether. The collected ethyl ether was dried using MgSO₄, the residue obtained by evaporating the solvent was separated and purified by silica gel column chromatography, to thereby obtain 2.00 g (yield: 70%) of Compound 38-2. The resulting compound was identified by LC-MS. C₂₁H₁₉N: M⁺285.3

### Synthesis of Intermediate 38-3

2.85 g (10 mmol) of Intermediate 38-2 and 4.29 g (30 mmol) of CuBr were dissolved in a 48% hydrobromic acid aqueous solution (10 ml), and then 2.07 g (in H₂O) of NaNO₂ was slowly added thereto at 0° C. The resultant was stirred for 5 hours at room temperature, and then, 3 g of Na₂S₂O₃ was dissolved in water and added thereto, followed by three times of washing using DCM (30 ml). The washed DCM layer was dried using MgSO₄ and then dried under reduced pressure to obtain a product, which was then separated and purified by silica gel column chromatography, to thereby obtain 2.44 g (yield: 70%) of Intermediate 38-3. The resulting compound was identified by LC-MS. C₂₁H₁₇Br: M⁺349.2

### Synthesis of Compound 38

3.49 g (10.0 mmol) of Intermediate 38-3, 5.47 g (10 mmol) of Intermediate 1-2, 0.46 g (0.5 mmol) of tris(dibenzylideneacetone)dipalladium(0) (Pd₂dba₃), 0.24 g (1 mmol) of P(t-Bu)₃, and 2.88 g (30 mmol) of sodium tert-butoxide were dissolved in 60 ml of toluene, and then stirred for 3 hours at 80° C. The reaction solution was cooled at room temperature, and then, 40 ml of water added thereto, followed by three times of an extraction process using 50 ml of ethyl ether. The collected ethyl ether was dried using MgSO₄, the residue obtained by evaporating the solvent was separated and purified by silica gel column chromatography, to thereby obtain 5.71 g (yield: 70%) of Compound 38. The resulting compound was identified by MS/FAB and 1H NMR.

### Synthesis Example 8: Synthesis of Compound 45

Compound 45 was synthesized in the same manner as in Synthesis Example of Compound 1, except that bicyclo[2.2.1]heptan-2-one was used instead of adamantan-2-one. The resulting compound was identified by MS/FAB and 1H NMR.

### Synthesis Example 9: Synthesis of Compound 57

Compound 57 was synthesized in the same manner as in Synthesis Example of Compound 13, except that bicyclo[2.2.1]heptan-2-one was used instead of adamantan-2-one. The resulting compound was identified by MS/FAB and 1H NMR.

### Synthesis Example 10: Synthesis of Compound 63

Compound 63 was synthesized in the same manner as in Synthesis Example of Compound 19, except that bicyclo[2.2.1]heptan-2-one was used instead of adamantan-2-one. The resulting compound was identified by MS/FAB and 1H NMR.

### Synthesis Example 11: Synthesis of Compound 65

Compound 65 was synthesized in the same manner as in Synthesis Example of Compound 21, except that bicyclo[2.2.1]heptan-2-one was used instead of adamantan-2-one. The resulting compound was identified by MS/FAB and 1H NMR.

### Synthesis Example 12: Synthesis of Compound 72

Compound 72 was synthesized in the same manner as in Synthesis Example of Compound 28, except that bicyclo[2.2.1]heptan-2-one was used instead of adamantan-2-one. The resulting compound was identified by MS/FAB and 1H NMR.

### Synthesis Example 13: Synthesis of Compound 74

Compound 74 was synthesized in the same manner as in Synthesis Example of Compound 30, except that bicyclo[2.2.1]heptan-2-one was used instead of adamantan-2-one. The resulting compound was identified by MS/FAB and 1H NMR.

### Synthesis Example 14: Synthesis of Compound 82

Compound 82 was synthesized in the same manner as in Synthesis Example of Compound 38, except that bicyclo[2.2.1]heptan-2-one was used instead of adamantan-2-one. The resulting compound was identified by MS/FAB and 1H NMR.

### Synthesis Example 15: Synthesis of Comparative Compound 2

Comparative Compound 2 was synthesized in the same manner as in Synthesis Example of Compound 1, except that iodobenzene was used instead of 2-bromo-9,9-dimethyl-9H-fluorene. The resulting compound was identified by MS/FAB and 1H NMR.

### Synthesis Example 16: Synthesis of Comparative Compound 3

Comparative Compound 3 (Compound 61) was synthesized in the same manner as in Synthesis Example of Comparative Compound 2, except that bicyclo[2.2.1]heptan-2-one was used instead of adamantan-2-one. The resulting compound was identified by MS/FAB and 1H NMR.

### Synthesis Example 17: Synthesis of Comparative Compound 4

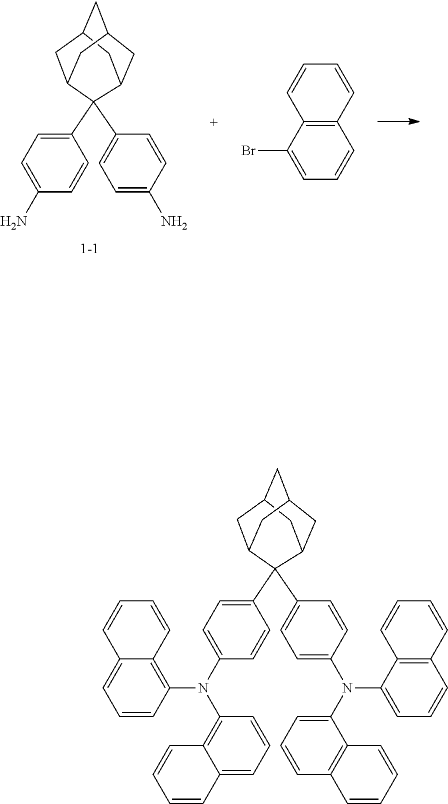 [SMILES: Nc1ccc(C2(c3ccc(N)cc3)C3CC4CC(C3)CC2C4)cc1 ; Brc1cccc2ccccc12 ; c1ccc2c(N(c3ccc(C4(c5ccc(N(c6cccc7ccccc67)c6cccc7ccccc67)cc5)C5CC6CC(C5)CC4C6)cc3)c3cccc4ccccc34)cccc2c1]

318 g (10.0 mmol) of Intermediate 1-1, 9.11 g (44 mmol) of 1-bromonaphthalene, 1.84 g (2 mmol) of tris(dibenzylideneacetone)dipalladium(0) (Pd₂dba₃), 0.96 g (4 mmol) of P(t-Bu)3, and 11.5 g (120 mmol) of sodium tert-butoxide were dissolved in 60 ml of toluene, and then stirred for 3 hours at 80° C. The reaction solution was cooled at room temperature, and then, 40 ml of water added thereto, followed by three times of an extraction process using 50 ml of ethyl ether. The collected ethyl ether was dried using MgSO₄, the residue obtained by evaporating the solvent was separated and purified by silica gel column chromatography, to thereby obtain 5.76 g (yield: 70%) of Comparative Compound 4. The resulting compound was identified by MS/FAB and 1H NMR.

Table 1 shows MS/FAB and ¹H NMR results of a compound prepared according to each Synthesis Example.

TABLE 1

| Com- | | MS/FAB | |
|---|---|---|---|
| pound | ¹H NMR (CDCl₃, 400 MHz) | found | calc. |
| Com- | 7.90-7.86(m, 2H), 7.55(d, 1H), 7.38- | 7939.22 | 739.02 |
| pound 1 | 7.00(m, 27H), 2.17(m, 2H), 1.72-1.66(m, | | |
| | 10H), 1.45-1.20(m, 4H), 1.07-1.01(m, 4H) | | |
| Com- | 8.03-7.98(m, 2H), 7.80(d, 1H), 7.54(d, | 712.99 | 712.94 |
| pound 13 | 1H), 7.39-7.00(m, 25H), 6.91(d, 1H), | | |
| | 2.17(m, 2H), 1.72-1.66(m, 4H), 1.45- | | |
| | 1.20(m, 4H), 1.07-1.01(m, 4H) | | |
| Com- | 8.45(m, 1H), 7.95-7.93(d, 2H), 7.85(d, | 729.25 | 729.00 |
| pound 19 | 1H), 7.56-7.41(m, 3H), 7.24-7.00(m, | | |
| | 23H), 2.17(m, 2H), 1.72-1.66(m, 4H), | | |
| | 1.45-1.20(m, 4H), 1.07-1.01(m, 4H) | | |
| Com- | 7.90-7.86(m, 2H), 7.55(d, 1H), 7.38- | 821.28 | 821.17 |
| pound 21 | 7.00(m, 26H), 2.72(m, 1H), 2.17(m, | | |
| | 2H), 1.86-1.20(m, 24H), 1.07-1.01(m, 4H) | | |
| Com- | 8.10(d, 1H), 7.90-7.86(m, 2H), 7.55(d, | 815.33 | 815.12 |
| pound 28 | 1H), 7.43-7.00(m, 30H), 2.17(m, 2H), | | |
| | 1.72-1.66(m, 10H), 1.45-1.20(m, 4H), | | |
| | 1.07-1.01(m, 4H) | | |
| Com- | 8.10(d, 1H), 7.90-7.86(m, 2H), 7.55(d, | 815.33 | 815.12 |
| pound 30 | 1H), 7.43-7.00(m, 30H), 2.17(m, 2H), | | |
| | 1.72-1.66(m, 10H),1.45-1.20(m, 4H), | | |
| | 1.07-1.01(m, 4H) | | |
| Com- | 8.14(s, 1H), 7.90(d, 1H), 7.55-7.52(m, | 815.33 | 815.12 |
| pound 38 | 2H), 7.43-7.38(m, 4H), 7.24-7.00(m, | | |
| | 26H), 2.17(m, 2H), 1.72-1.66(m, 10H), | | |
| | 1.45-1.20(m, 4H), 1.07-1.01(m, 4H) | | |
| Com- | 7.90-7.86(m, 2H), 7.55(d, 1H), 7.38- | 698.99 | 698.95 |
| pound 45 | 7.00(m, 27H), 2.20-2.09(m, 4H), 1.88- | | |
| | 1.84(m, 2H), 1.69(s, 6H), 1.58-1.31(m, | | |
| | 4H) | | |
| Com- | 8.03-7.98(m, 2H), 7.80(d, 1H), 7.54(d, | 672.95 | 672.82 |
| pound 57 | 1H), 7.39-7.00(m, 25H), 6.91(d, 1H), | | |
| | 2.20-2.09(m, 4H), 1.88-1.84(m, 2H), | | |
| | 1.58-1.31(m, 4H) | | |
| Com- | 8.45(m, 1H), 7.95-7.93(d, 2H), 7.85(d, | 688.99 | 688.93 |
| pound 63 | 1H), 7.56-7.41(m, 3H), 7.24-7.00(m, | | |
| | 23H), 2.20-2.09(m, 4H), 1.88-1.84(m, | | |
| | 2H), 1.58-1.31(m, 4H) | | |
| Com- | 7.90-7.86(m, 2H), 7.55(d, 1H), 7.38- | 781.30 | 781.10 |
| pound 65 | 7.00(m, 26H), 2.72(m, 1H), 2.20-2.09(m, | | |
| | 4H), 1.88-1.84(m, 6H), 1.69(s, 6H) | | |
| | 1.58-1.31(m, 10H) | | |
| Com- | 8.10(d, 1H), 7.90-7.86(m, 2H), 7.55(d, | 775.20 | 775.05 |
| pound 72 | 1H), 7.43-7.00(m, 30H), 2.20-2.09(m, | | |
| | 4H), 1.88-1.84(m, 2H), 1.69(s, 6H), | | |
| | 1.58-1.31(m, 4H) | | |
| Com- | 8.10(d, 1H), 7.90-7.86(m, 2H), 7.55(d, | 775.20 | 775.05 |
| pound 74 | 1H), 7.43-7.00(m, 30H), 2.20-2.09(m, | | |
| | 4H), 1.88-1.84(m, 2H), 1.69(s, 6H), | | |
| | 1.58-1.31(m, 4H) | | |
| Com- | 8.14(s, 1H), 7.90(d, 1H), 7.55-7.52(m, | 775.20 | 775.05 |
| pound 82 | 2H), 7.43-7.38(m, 4H), 7.24-7.00(m, | | |
| | 26H), 2.20-2.09(m, 4H), 1.88-1.84(m, | | |
| | 2H), 1.69(s, 6H), 1.58-1.31(m, 4H) | | |
| Compar- | 7.24-7.00(m, 28H), 2.17(m, 2H), 1.72- | 622.97 | 622.86 |
| ative | 1.66(m, 4H), 1.45-1.20(m, 4H), 1.07- | | |
| Com- | 1.01(m, 4H) | | |
| pound 2 | | | |
| Compar- | 7.24-7.00(m, 28H), 2.20-2.09(m, 4H), | 582.89 | 582.79 |
| ative | 1.88-1.84(m, 2H), 1.58-1.31(m, 4H) | | |
| Com- | | | |
| pound 3 | | | |
| Compar- | 8.22(d, 4H), 8.15(d, 4H), 7.81(d, 4H), | 823.20 | 823.10 |
| ative | 7.63-7.50(m, 16H), 7.17-1.14(m, 8H), ), | | |
| Com- | 2.17(m, 2H), 1.72-1.66(m, 4H), 1.45- | | |
| pound 4 | 1.20(m, 4H), 1.07-1.01(m, 4H) | | |

### Evaluation Example 1

LUMO and HOMO values of compounds of Synthesis Examples were measured using methods described in Table 2, and by using the DFT method of the Gaussian 09 program (with the structure optimization at the level of B3LYP, 6-311 G(d,p)), Ti, dipole, and MLCT values of Compounds of Synthesis Examples were calculated. The results are shown in Table 3.

TABLE 2

| | |
|---|---|
| HOMO | By using cyclic voltammetry (CV) (electrolyte: 0.1M |
| energy | Bu₄NPF₆/solvent: dimethylforamide (DMF)/electrode: 3- |
| level | electrode system (working electrode: GC, reference |
| evaluation | electrode: Ag/AgCl, and auxiliary electrode: Pt)), the |
| method | potential (V)-current (A) graph of each compound was |
| | obtained, and then, from the oxidation onset of the graph, |
| | the HOMO energy level of each compound was calculated. |
| LUMO | By using cyclic voltammetry (CV) (electrolyte: 0.1M |
| energy | BU4NPF6/solvent: dimethylforamide (DMF)/electrode: 3- |
| level | electrode system (working electrode: GC, reference |
| evaluation | electrode: Ag/AgCl, and auxiliary electrode: Pt)), the |
| method | potential (V)-current (A) graph of each compound was |
| | obtained, and then, from the reduction onset of the graph, |
| | the LUMO energy level of each compound was calculated. |

TABLE 3

| Compound No. | HOMO (eV) | LUMO (eV) |
|---|---|---|
| 1 | −5.18 | −1.87 |
| 13 | −5.13 | −1.84 |
| 19 | −5.14 | −1.84 |
| 21 | −5.15 | −1.84 |
| 28 | −5.15 | −1.86 |
| 30 | −5.15 | −1.85 |
| 38 | −5.19 | −1.96 |
| 45 | −5.13 | −2.05 |
| 57 | −5.12 | −1.82 |
| 63 | −5.13 | −1.82 |
| 65 | −5.14 | −1.85 |
| 72 | −5.14 | −1.85 |
| 74 | −5.14 | −1.86 |
| 82 | −5.18 | −1.90 |
| Comparative Compound 2 | −5.20 | −1.88 |
| Comparative Compound 3 | −5.20 | −1.88 |
| Comparative Compound 4 | −5.20 | −1.92 |

### Example 1

As an anode, an ITO-deposited substrate was cut to a size of 50 mm×50 mm×0.7 mm, sonicated with isopropyl alcohol and pure water each for 5 minutes, and then cleaned irradiation of ultraviolet rays and exposure of ozone thereto for 30 minutes. Then, the substrate was loaded onto a vacuum deposition apparatus.

Compound 2-TNATA was vacuum-deposited on the ITO substrate to form a hole injection layer having a thickness of 600 Å, and then Compound 1 was vacuum-deposited on the hole injection layer to form a hole transport layer having a thickness of 300 Å.

9,10-di(naphthalen-2-yl)anthracene (hereinafter, referred to as DNA) which is a known blue fluorescent host in the art and 4,4′-bis[2-(4-(N,N-diphenylamino)phenyl)vinyl]biphenyl (hereinafter, referred to as DPAVBi) which is a known blue phosphorescent dopant compound in the art were co-deposited on the hole transport layer at a weight ratio of 98:2 to form an emission layer having a thickness of 300 Å.

Alq₃ was deposited on the emission layer to form an electron transport layer having a thickness of 300 Å, and then LiF as an alkali metal halide was deposited on the electron transport layer to form an electron injection layer having a thickness of 10 Å, and Al was vacuum-deposited thereon to form a cathode electrode having a thickness of 3,000 Å, to form an LiF/AI electrode, thereby completing manufacture of a light-emitting device.

### Examples 2 to 14

Light-emitting devices were manufactured in the same manner as in Example 1, except that a hole transport material was changed as in Table 4 below.

### Example 15

A light-emitting device was manufactured in the same manner as in Example 1, except that Compound 1 was vacuum-deposited on the hole injection layer to form a first hole transport layer having a thickness of 100 Å, HT1 was vacuum-deposited on the first hole transport layer to form a second hole transport layer having a thickness of 100 Å, and Compound 1 was vacuum-deposited on the second hole transport layer to form a third hole transport layer having a thickness of 100 Å.

### Example 16

A light-emitting device was manufactured in the same manner as in Example 1, except that Compound 21 was vacuum-deposited on the hole injection layer to form a first hole transport layer having a thickness of 100 Å, HT1 was vacuum-deposited on the first hole transport layer to form a second hole transport layer having a thickness of 100 Å, and Compound 21 was vacuum-deposited on the second hole transport layer to form a third hole transport layer having a thickness of 100 Å.

### Comparative Examples 1 to 4

Light-emitting devices were manufactured in the same manner as in Example 1, except that a hole transport material was changed as in Table 4 below.

A voltage was supplied so that the light-emitting devices manufactured according to Examples 1 to 17 and Comparative Examples 1 to 3 had a current density of 50 mA/cm². Driving voltage (V), luminance (cd/m²), luminescence efficiency (cd/A), emission color, emission wavelength (nm), and half lifespan (hr @ 100 mA/cm²) were each measured using the Keithley MU 236 and the luminance meter PR650, and the results are shown in Table 4 below.

TABLE 4

| | | | | | | | Half |
|---|---|---|---|---|---|---|---|
| | | | | | | | lifespan |
| | Hole transport | Driving | Current | Luminance | Efficiency | Emission | (hr @ 100 |
| | material | voltage | density | (cd/m²) | (cd/A) | color | mA/cm²) |
| Example 1 | Compound 1 | 4.95 | 50 | 3125 | 6.25 | Blue | 610 |
| Example 2 | Compound 13 | 5.05 | 50 | 3160 | 6.32 | Blue | 650 |
| Example 3 | Compound 19 | 5.02 | 50 | 3225 | 6.45 | Blue | 450 |
| Example 4 | Compound 21 | 4.95 | 50 | 3210 | 6.42 | Blue | 660 |
| Example 5 | Compound 28 | 4.92 | 50 | 3215 | 6.43 | Blue | 630 |
| Example 6 | Compound 30 | 4.92 | 50 | 3250 | 6.50 | Blue | 550 |
| Example 7 | Compound 38 | 4.90 | 50 | 3125 | 6.25 | Blue | 630 |
| Example 8 | Compound 45 | 4.92 | 50 | 3260 | 6.52 | Blue | 580 |
| Example 9 | Compound 57 | 5.00 | 50 | 3210 | 6.42 | Blue | 560 |
| Example 10 | Compound 63 | 5.01 | 50 | 3280 | 6.56 | Blue | 600 |
| Example 11 | Compound 65 | 4.93 | 50 | 3280 | 6.56 | Blue | 610 |
| Example 12 | Compound 72 | 4.93 | 50 | 3160 | 6.32 | Blue | 520 |
| Example 13 | Compound 74 | 4.91 | 50 | 3260 | 6.52 | Blue | 580 |
| Example 14 | Compound 82 | 4.90 | 50 | 3310 | 6.62 | Blue | 510 |
| Example 15 | Compound 1/HT1/ | 5.15 | 50 | 3270 | 6.54 | Blue | 715 |
| | Compound 1 | | | | | | |
| Example 16 | Compound 21/HT1/ | 5.15 | 50 | 3310 | 6.62 | Blue | 750 |
| | Compound 21 | | | | | | |
| Comparative | Comparative | 7.01 | 50 | 2645 | 5.29 | Blue | 258 |
| Example 1 | Compound 1 | | | | | | |
| Comparative | Comparative | 5.25 | 50 | 3015 | 6.03 | Blue | 500 |
| Example 2 | Compound 2 | | | | | | |
| Comparative | Comparative | 5.23 | 50 | 3025 | 6.05 | Blue | 510 |
| Example 3 | Compound 3 | | | | | | |
| Comparative | Comparative | 5.21 | 50 | 3050 | 6.10 | Blue | 450 |
| Example 4 | Compound 4 | | | | | | |

Structures of hole transport materials used in Comparative Examples 1 to 4 are as follows.

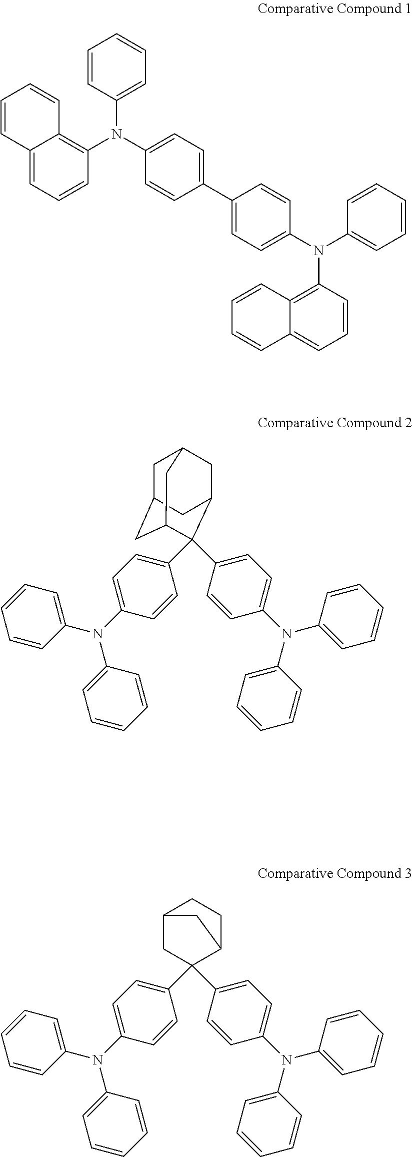 [SMILES: c1ccc(N(c2ccc(-c3ccc(N(c4ccccc4)c4cccc5ccccc45)cc3)cc2)c2cccc3ccccc23)cc1 ; c1ccc(N(c2ccccc2)c2ccc(C3(c4ccc(N(c5ccccc5)c5ccccc5)cc4)C4CC5CC(C4)CC3C5)cc2)cc1 ; c1ccc(N(c2ccccc2)c2ccc(C3(c4ccc(N(c5ccccc5)c5ccccc5)cc4)CC4CCC3C4)cc2)cc1]

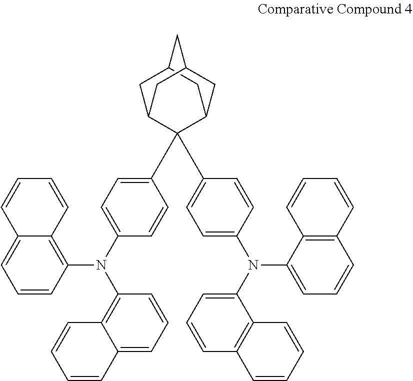 [SMILES: c1ccc2c(N(c3ccc(C4(c5ccc(N(c6cccc7ccccc67)c6cccc7ccccc67)cc5)C5CC6CC(C5)CC4C6)cc3)c3cccc4ccccc34)cccc2c1]

Referring to Table 4, it was confirmed that the light-emitting device according to each Example had excellent characteristics in terms of driving voltage (V), luminance (cd/m²), efficiency (cd/A), and/or half lifespan, as compared to the hole transport materials of Comparative Examples 1 to 4.

Also, in the light-emitting devices of Examples 15 and 16, Compounds 1 and 21, both having a small refractive index, were included in the first hole transport layer and the third hole transport layer, respectively, and HT1 with a relatively high refractive index was included in the second hole transport layer. As a result, it was found that the resonance effect and hole transport capability were improved in a balanced way, and the half lifespan was further improved.

By using the diamine-based compound, a light-emitting device having high efficiency and long lifespan and a high-quality electronic apparatus including the same may be manufactured.

The use of “may” when describing embodiments of the present disclosure refers to “one or more embodiments of the present disclosure.”

As used herein, the term “substantially,” “about,” and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this disclosure is intended to include all higher numerical limitations subsumed therein. Accordingly, Applicant reserves the right to amend this disclosure, including the claims, to expressly recite any sub-range subsumed within the ranges expressly recited herein.

The light-emitting device and/or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

It should be understood that embodiments described herein should be considered in a descriptive sense only and not for purposes of limitation. Descriptions of features or aspects within each embodiment should typically be considered as available for other similar features or aspects in one or more embodiments. While one or more embodiments have been described with reference to the figures, it will be understood by those of ordinary skill in the art that various changes in form and details may be made therein without departing from the spirit and scope of the present disclosure as defined by the following claims, and equivalents thereof.

What is claimed is:

1. A light-emitting device comprising:
a first electrode;
a second electrode facing the first electrode;
an interlayer arranged between the first electrode and the second electrode and comprising an emission layer; and
a diamine-based compound represented by Formula 1: 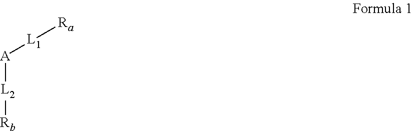
wherein, in Formula 1,
A is 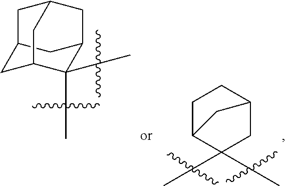 [SMILES: *C1(*)C2CC3CC(C2)CC1C3 ; *C1(*)CC2CCC1C2]
L₁ and L₂ are each independently a single bond, *—C(R₅)(R₆)—*′, *—C(R₅)═*′, *═C(R₅)—*′, *—C(R₅)═C(R₆)—*′, *—C(═O)—*′, *—C(═S)—*′, *—C≡C—*′, *—B(R₅)—*′, *—N(R₅)—*′, *—P(R₅)—*′, *—Si(R₅)(R₆)—*′, *—P(═O)(R₅)—*′, *—S(═O)—*′, *—S(═O)₂—*′, or *—Ge(R₅)(R₆)—*′, in Formula 1, [symbol] , *, and *′ each indicate a binding site to a neighboring atom, and
Rₐ and R_{b} are each independently a group represented by Formula 1-1, 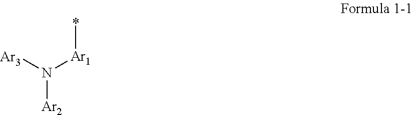
wherein, in Formula 1-1,
Ar₁ is a divalent linking group of a C₆-C₆₀ aryl group unsubstituted or substituted with at least one R₁₀ₐ or a divalent linking group of a C₃-C₆₀ heteroaryl group unsubstituted or substituted with at least one R₁₀ₐ, and * indicates a binding site to an atom included in A,
Ar₂ and Ar₃ are each independently a C₆-C₆₀ aryl group unsubstituted or substituted with at least one R₁₀ₐ or a C₃-C₆₀ heteroaryl group unsubstituted or substituted with at least one R₁₀ₐ, and
at least one group of Ar₂ and Ar₃ is a group represented by Formula 1-2, 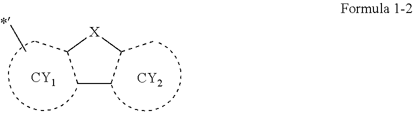
wherein X is one of O, S, N(Q₁), P(Q₁), C(Q₁)(Q₂), and Si(Q₁)(Q₂),
CY₁ and CY₂ are each independently a C₆-C₃₀ aryl group unsubstituted or substituted with at least one R₁₀ₐ or a C₃-C₃₀ heteroaryl group unsubstituted or substituted with at least one R₁₀ₐ,
*′ indicates a binding site to an atom included in the group represented by Formula 1-1,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁, Q₂, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and
Q₁ and Q₂ are optionally bonded together to form a C₃-C₆₀ carbocyclic group unsubstituted or substituted with at least one R₁₀ₐ or a C₁-C₆₀ heterocyclic group unsubstituted or substituted with at least one R₁₀ₐ.

2. The light-emitting device of claim 1, wherein the interlayer comprises the diamine-based compound represented by Formula 1.

3. The light-emitting device of claim 1, wherein the first electrode is an anode,
the second electrode is a cathode,
the interlayer further comprises a hole transport region located between the first electrode and the emission layer, and an electron transport region located between the emission layer and the second electrode,
the hole transport region comprises a hole injection layer, a first hole transport layer, an emission auxiliary layer, an electron blocking layer, or any combination thereof, and
the electron transport region comprises a hole blocking layer, an electron transport layer, an electron injection layer, or any combination thereof.

4. The light-emitting device of claim 3, wherein the hole transport region comprises the diamine-based compound represented by Formula 1.

5. The light-emitting device of claim 3, wherein the first hole transport layer comprises the diamine-based compound represented by Formula 1.

6. The light-emitting device of claim 2, wherein the hole transport region further comprises a second hole transport layer and a third hole transport layer, the second hole transport layer comprises an amine-based compound represented by N(Ar₂₁)(Ar₂₂)(Ar₂₃),
the third hole transport layer comprises the same diamine-based compound as the interlayer, and
Ar₂₁ to Ar₂₃ are each independently a C₆-C₆₀ aryl group unsubstituted or substituted with at least one R₁₀ₐ, a C₃-C₆₀ heteroaryl group unsubstituted or substituted with at least one R₁₀ₐ, or a C₆-C₆₀ condensed polycyclic group unsubstituted or substituted with at least one R₁₀ₐ.

7. The light-emitting device of claim 6, wherein a refractive index of the second hole transport layer is greater than a refractive index of the first hole transport layer.

8. The light-emitting device of claim 6, wherein a refractive index of the first hole transport layer is from 1.4 to 1.7, and
a refractive index of the second hole transport layer is from 1.8 to 2.0.

9. The light-emitting device of claim 6, wherein the amine-based compound is Compound HT1:

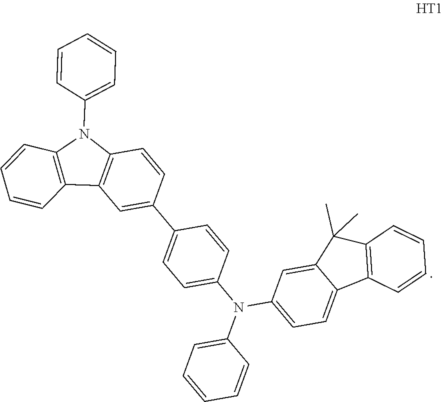 [SMILES: CC1(C)c2ccccc2-c2ccc(N(c3ccccc3)c3ccc(-c4ccc5c(c4)c4ccccc4n5-c4ccccc4)cc3)cc21]

10. An electronic apparatus comprising the light-emitting device according to claim 1.

11. The electronic apparatus of claim 10, further comprising a thin-film transistor, wherein
the thin-film transistor comprises a source electrode and a drain electrode, and
the first electrode of the light-emitting device is electrically connected to at least one of the source electrode or the drain electrode of the thin-film transistor.

12. The electronic apparatus of claim 10, further comprising a color filter, a color conversion layer, a touch screen layer, a polarizing layer, or any combination thereof.

13. A diamine-based compound represented by Formula 1: 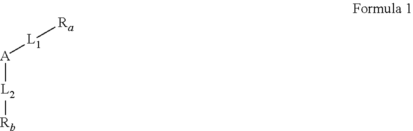
wherein, in Formula 1,
A is 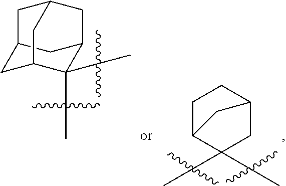 [SMILES: *C1(*)C2CC3CC(C2)CC1C3 ; *C1(*)CC2CCC1C2]
L₁ and L₂ are each independently a single bond, *—C(R₅)(R₆)—*′, *—C(R₅)═*′, *═C(R₅)—*′, *—C(R₅)═C(R₆)—*′, *—C(═O)—*′, *—C(═S)—*′, *—C≡C—*′, *—B(R₅)—*′, *—N(R₅)—*′, *—P(R₅)—*′, *—Si(R₅)(R₆)—*′, *—P(═O)(R₅)—*′, *—S(═O)—*′, *—S(═O)₂—*′, or *—Ge(R₅)(R₆)—*′, in Formula 1, [symbol] , *, and *′ each indicate a binding site to a neighboring atom, and
Rₐ and R_{b} are each independently a group represented by Formula 1-1, 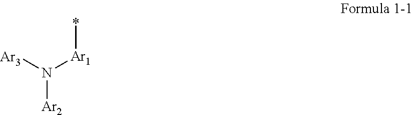
wherein, in Formula 1-1,
Ar₁ is a divalent linking group of a C₆-C₆₀ aryl group unsubstituted or substituted with at least one R₁₀ₐ or a divalent linking group of a C₃-C₆₀ heteroaryl group unsubstituted or substituted with at least one R₁₀ₐ, and * indicates a binding site to an atom included in A,
Ar₂ and Ar₃ are each independently a C₆-C₆₀ aryl group unsubstituted or substituted with at least one R₁₀ₐ or a C₃-C₆₀ heteroaryl group unsubstituted or substituted with at least one R₁₀ₐ, and
at least one group of Ar₂ and Ar₃ is a group represented by Formula 1-2, 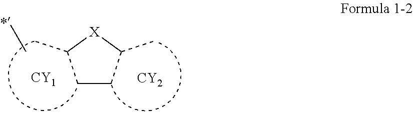
wherein X is one of O, S, N(Q₁), P(Q₁), C(Q₁)(Q₂), and Si(Q₁)(Q₂),
CY1 and CY₂ are each independently a C₆-C₃₀ aryl group unsubstituted or substituted with at least one R₁₀ₐ or a C₃-C₃₀ heteroaryl group unsubstituted or substituted with at least one R₁₀ₐ,
*′ indicates a binding site to an atom included in the group represented by Formula 1-1,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁, Q₂, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and
Q₁ and Q₂ are optionally bonded together to form a C₃-C₆₀ carbocyclic group unsubstituted or substituted with at least one R₁₀ₐ or a C₁-C₆₀ heterocyclic group unsubstituted or substituted with at least one R₁₀ₐ.

14. The diamine-based compound of claim 13, wherein Rₐ and R_{b} are different from each other.

15. The diamine-based compound of claim 13, wherein Ar₂ and Ar₃ are different from each other.

16. The diamine-based compound of claim 13, wherein at least one group of Ar₂ and Ar₃ is a group represented by Formulae 1-2-1 to 1-2-4: 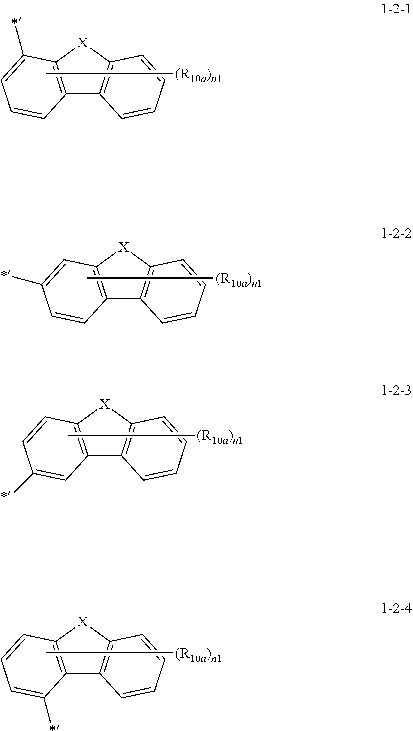
wherein, in Formulae 1-2-1 to 1-2-4, and
n1 is an integer from 0 to 7.

17. A diamine-based compound comprising a condensed polycyclic group and an aromatic substituted amine group, wherein
the aromatic substituted amine group comprises a fluorene group unsubstituted or substituted with at least one R₁₀ₐ or a fluorene derivative group unsubstituted or substituted with at least one R₁₀ₐ,
an energy level of a lowest unoccupied molecular orbital (LUMO) is from −5.3 eV to −5.1 eV,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁, Q₂, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and
Q₁ and Q₂ are optionally bonded together to form a C₃-C₆₀ carbocyclic group unsubstituted or substituted with at least one R₁₀ₐ or a C₁-C₆₀ heterocyclic group unsubstituted or substituted with at least one R₁₀ₐ.

18. The diamine-based compound of claim 17, wherein the condensed polycyclic group is one of an adamantanyl group, a norbornanyl group, a norbornenyl group, a bicyclo[1.1.1]pentyl group, a bicyclo[2.1.1]hexyl group, a bicyclo[2.2.2]octyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.2]octyl group, a mono(C₁-C₂₀ alkyl)adamantanyl group, a di(C₁-C₂₀ alkyl)adamantanyl group, a mono(C₁-C₂₀ alkyl)norbornanyl group, a di(C₁-C₂₀ alkyl)norbornanyl group, a mono(C₁-C₂₀ alkyl)norbornenyl group, and a di(C₁-C₂₀ alkyl)norbornenyl group.

19. The diamine-based compound of claim 17, wherein
the fluorene derivative group comprises at least one of a carbazole moiety, a dibenzosilole moiety, a dibenzothiophene moiety, and a dibenzofuran moiety.

20. The diamine-based compound of claim 17, wherein the condensed polycyclic group and the amine group are linked together through at least one aromatic linking group,
the aromatic linking group is a divalent linking group of a C₆-C₆₀ aryl group unsubstituted or substituted with at least one R₁₀ₐ or a divalent linking group of a C₃-C₆₀ heteroaryl group unsubstituted or substituted with at least one R₁₀ₐ.
